(19) United States
(12) Patent Application Publication
(10) Pub. No.: US 2022/0148730 A1
(43) Pub. Date: May 12, 2022

(54) Inhaler System

(71) Applicant: Norton (Waterford) Limited, Waterford (IE)

(72) Inventors: Michael Naumov, Beer-Yakov (IL); Yaron Nir, Nes Ziona (IL)

(73) Assignee: Norton (Waterford) Limited, Waterford (IE)

(21) Appl. No.: 17/505,821

(22) Filed: Oct. 20, 2021

Related U.S. Application Data
(60) Provisional application No. 63/094,509, filed on Oct. 21, 2020.

(51) Int. Cl. G16H 50/20; G16H 50/30; G16H 20/13

(52) U.S. Cl. CPC G16H 50/20; G16H 20/13; G16H 50/30

(57) ABSTRACT

A system may include a plurality of inhalers, where each inhaler comprising medicament, a processor, memory, and a transmitter, multiple processing modules that may reside at least partially on a user device, a digital health platform (DHP) that is configured to receive and aggregate inhaler data from inhalers that are associated with a plurality of different users and a plurality of different medicament types. The DHP may be configured to train a machine learning algorithm using training data via a supervised or an unsupervised learning method, wherein the training data comprises the time and the one or more inhalation parameters associated with each of the plurality of usage events. The DHP also configured to generate a compliance score, a future compliance score, and/or a risk score using the trained machine learning algorithm, and cause a display device to generate a notification indicating the score for the user.

### CROSS-REFERENCE TO RELATED APPLICATIONS

This application claims priority to U.S. patent application No. 63/094,509, filed Oct. 21, 2020, which is incorporated herein by reference in its entirety.

### BACKGROUND

Drug delivery devices facilitate the delivery of medication into a patient's body via various routes of administration. Typical routes of administration include oral, topical, sublingual inhalation, injection, and the like. The devices may be used to deliver medications for the treatment various diseases, ailments, and medical conditions. Inhalers, for example, may be used to treat asthma, chronic obstructive pulmonary disease (COPD) and/or cystic fibrosis (CF). While drug delivery devices are designed to deliver an appropriate dose of medication to a patient as part of a therapeutic treatment, the effectiveness of a particular treatment may be influenced by non-physiological factors, such as the patient's adherence and compliance.

In the context of a drug therapy, adherence may refer to the degree to which a patient is following a prescribed dosing regimen. For example, if the patient's prescription calls for two doses each day, and the patient is taking two doses per day, the patient may be considered 100% adherent. If the patient is only taking one dose per day, he or she may be deemed only 50% adherent. In the latter case, the patient may not be receiving the treatment prescribed by his or her doctor, which may negatively affect the efficacy of the therapeutic treatment.

Compliance may refer to a patient's technique when using a particular drug delivery device. If the patient is using the device in a manner that is recommended by a doctor or by a manufacturer, the device is likely to deliver the desired dose of medication and the patient may be deemed compliant. However, if the device is not being used properly during drug administration, the device's ability to deliver a proper dose of medication may be compromised. As such, the patient may be deemed non-compliant. In the case of an inhalation device or inhaler, for example, the patient may need to achieve a minimum inspiratory effort to ensure a full dose of medication is delivered from the device into the patient's lungs. For some patients, such as children and the elderly, meeting the requirements for full compliance may be difficult, for example, due to physical limitations, such as limited lung function. Accordingly, like adherence, failing to achieve full compliance may reduce the effectiveness of a prescribed treatment.

A patient's ability to achieve full compliance may be further complicated by certain physical properties of the medication. For example, some respiratory medications may consist of fine particles and/or may lack any odor or taste. Thus, a patient using an inhaler may not be able to correct a non-compliant use because he or she may not be able to immediately detect or sense that medication is being inhaled and/or know whether the amount of inhaled medication complies with the prescription.

Further, many respiratory diseases, such as asthma and COPD, are life-long conditions where treatment involves the long-term administration of medicaments to manage the patients' symptoms and to decrease the risks of irreversible changes. There is currently no cure for diseases like asthma and COPD. Treatment takes two forms. First, a maintenance aspect of the treatment is intended to reduce airway inflammation and, consequently, control symptoms in the future. The maintenance therapy is typically provided by inhaled corticosteroids, alone or in combination with long-acting bronchodilators and/or muscarinic antagonists. Secondly, there is also a rescue (or reliever) aspect of the therapy, where patients are given rapid-acting bronchodilators to relieve acute episodes of wheezing, coughing, chest tightness and shortness of breath.

Sufferers of respiratory diseases may thus be prescribed more than one medication, such as more than one inhaled medication, for controlling their symptoms. The sufferer may alternatively or additionally make use of a plurality of inhalers, each being used at different times/locations, which all deliver the same inhaled medicament. There is a growing desire to monitor administration of such medicaments in ways which are reliable, and convenient from the point of view of the sufferer. Further, such monitoring is also desirable for the patient's health care provider, who requires a simple and cohesive manner to assess each patient's adherence and compliance, and view data that indicates the highest risk of exacerbation or indicates that the patient's treatment regimen should be altered.

Systems may be designed to monitor a patient's adherence to a prescribed dosing regimen. For example, systems may be designed to monitor a patient's adherence by detecting administrations of a medicament by the patient and confirm that the patient is conforming to the prescribed dosing regimen. In order to detect administrations of the medicament, certain components may be added to or included in the drug delivery device used to administer the medicament. For example, components may be added to detect events or actuations at the drug delivery device that are indicative of an administration of the medicament by the drug delivery device. In addition, communication components may be added to the drug delivery device so that certain information associated with a respective administration of medicament by the drug delivery device (e.g., time of administration, number of remaining doses, etc.) can be transmitted to and subsequently stored at and/or analyzed by one or more external devices. Often, these components are add-on components, such as after-market processing and/or communication components that can be attached to a drug delivery device.

However, while these systems may be used to detect and monitor administrations of a drug delivery device and, by extension, measure a patient's adherence to (e.g., the degree to which a patient is following) a prescribed dosing regimen, they fail to measure or monitor the patient's compliance (e.g., the patient's technique when using a particular drug delivery device). For example, while these systems may detect and monitor administrations of medicament by the drug delivery device, they fail to measure the inhalation parameters (e.g., PIF, volume, etc.) associated with each administration event. That is, although these systems may be capable of monitoring a patient's adherence to a prescribed dosing regimen, they fail to measure inhalation parameters and thus lack any insight into a patient's compliance. In failing to measure inhalation parameters, these systems also fail to appreciate the state of the patient's respiratory disease (e.g., the likelihood that the patient is to experience an exacerbation event), such as the severity of an exacerbation and/or the patient's general lung health, and/or the patient's sensitivity to current or changing environmental conditions (e.g., heat, air quality, humidity, etc.).

Systems that are designed to monitor a patient's adherence to a prescribed dosing regimen may also provide feedback to the patient based on the patient's adherence. For example, these systems may provide notifications/alerts to the patient if they fail to follow the prescribed dosing regimen. In addition, these systems may attempt to predict the state of the patient's respiratory disease (e.g., the likelihood that the patient is to experience an exacerbation event) and/or the patient's sensitivity to current or changing environmental conditions (e.g., heat, air quality, humidity, etc.) based on their respective adherence. The state of the patient's respiratory disease and/or sensitivity to current or changing environmental conditions is, however, better predicted (e.g., predicted with a higher degree of accuracy) based the patient's compliance. As a result, systems that attempt to predict the state of a patient's respiratory disease and/or their sensitivity to current or changing environmental conditions based only on the patient's adherence may be inaccurate and/or unreliable. These shortcoming may be further exacerbated when a patient is prescribed multiple medicaments (e.g., a rescue medicament and a maintenance medicament) to treat a respiratory disease. For example, as each of the medicaments may be administered at different times, in response to different conditions, and/or for different purpose, the patient's compliance with respect to each type of medicament may affect the state of the patient's respiratory disease and/or their sensitivity to current or changing environmental conditions.

### SUMMARY

A system may include a plurality of inhalers, where each inhaler comprises medicament, a processor, memory, and a transmitter. Each inhaler is configured to determine usage events of the inhaler caused by a subject, such as an inhalation event (e.g., as detected by a sensor, such as a pressure sensor, acoustic sensor, a flow sensor, etc.) or an operation, such as the opening of a mouthpiece cover, the actuation of a switch (e.g., that is used to prime a dose of the medicament), or the detection of a particular movement of the inhaler, like shaking (e.g., as detected using an accelerometer). The inhaler may assign a time to each usage event. The inhaler is also configured to send the data indicating the usage events and the time of the usage events to another device, which in some examples is a mobile application residing on a user device. Although described as inhalers, in some examples, the system may include other medical devices in addition to or as an alternative to inhalers. Further, in some examples, the system may include smart add-on devices that include a processing module and/or a transmission module (e.g., a processor, memory, and transmitter) that are configured to attach to otherwise “dumb” inhalers instead of, or in addition to, including inhalers described herein that include a processing module and/or a transmission module (e.g., a processor, memory, and transmitter).

The system may also include a processing module that is configured to connect to, receive, and aggregate inhaler data from inhalers that are associated with a plurality of different users and a plurality of different medicament types, such as one or more rescue medicament types and/or one or more maintenance medicament types. In some examples, the rescue medicament type is albuterol (such as albuterol sulfate), and the maintenance medicament type is fluticasone (such as fluticasone propionate or fluticasone furoate) or salmeterol (such as salmeterol xinafoate) combined with fluticasone (such as fluticasone propionate or fluticasone furoate). This processing module may be referred to as a digital health platform (DHP), as will be described in more detail below. The DHP may include or be associated with a transceiver, memory, and a processor. For instance, the DHP may reside on one or more servers.

The DHP is configured to receive data relating to usage events and a time of each of the usage events (e.g., a time stamp for the usage event) for a plurality of inhalers, where the inhalers are associated with a plurality of different users. Each inhaler is associated with at least one user and one of a rescue medicament type or a maintenance medicament type. In some examples, the system may include mobile applications that are residing on user devices (e.g., such as a smartphone or tablet), and the DHP may receive the usage events and time stamps for the plurality of inhalers through a mobile application that resides on a user device (e.g., such as a smartphone or tablet). The system may include mobile applications that are residing on user devices (e.g., such as a smartphone or tablet). However, in other examples, the DHP may receive the usage events and time stamps directly from the inhalers themselves, for example, in instances where the inhalers include a cellular chipset that allows the inhalers to transmit the usage events and time stamps to the DHP.

The DHP is configured to determine the medicament type and the user associated with each of the plurality of inhalers (and in turn each usage event). In some examples, this is performed at a mobile application residing on the user's device, and then sent to the DHP along with the usage data and associated timestamp. In other examples, the DHP determines the medicament type and user associated with each inhaler.

Described herein are systems, methods, and computer readable mediums that when executed are configured to determine one or more individualized scores for a user, such as a compliance score, a future compliance score, and/or a risk score. The compliance score may indicate how compliant the user has been during usage events in a last predetermined number of days. For instance, in some examples, the compliance score may further indicates how adherent the user has been with respect to a dosing schedule associated with a maintenance medicament. The future compliance score may indicate an evaluation of anticipated future compliance of the user as it relates to one or more inhalers. The risk score may include the risk that the user suffers an exacerbation of a respiratory condition, such as asthma, chronic obstructive pulmonary disease (COPD), or cystic fibrosis (CF).

For instance, the DHP may be configured to receive a plurality of usage events associated with a plurality of different users. Each usage event may be associated with an inhaler, a medicament type, and a user of the plurality of different users. Each usage event may include a time associated with the usage event and one or more inhalation parameters of the usage event. The inhalation parameters may include any combination of a flow rate, a peak inspiratory flow (PIF), an inhaled volume, an inhalation duration, or a time-to-peak inhalation. For example, the inhalation parameters may include the PIF for the usage event and/or the inhalation volume for the usage event. The DHP may train a machine learning algorithm using training data via a supervised learning method or an unsupervised learning method. The training data may include the time and the one or more inhalation parameters associated with each of the plurality of usage events. The DHP may determine the compliance score, the future compliance score, and/or the risk score for a user using the trained machine learning algorithm. The DHP may be configured to cause a display device to generate a notification indicating the score for the user where, for example, the display device may be associated with the user (e.g., at the user's phone) and/or the user's health care provider.

The training data may include an adherence ratio that indicates the user's adherence to the dosing schedule for a maintenance medicament type for each user of the plurality of users that is associated with at least one maintenance usage event. The adherence may be determined based on a comparison between a number of maintenance usage events of the user over a predetermined period of time and a number of maintenance usage events indicated by the dosing schedule for the predetermined period of time. The training data may include a user's frequency of rescue usage events for each user of the plurality of users that is associated with at least one rescue usage event. The user's frequency of rescue usage events may include a comparison between a number of rescue usage events of the user over a predetermined period of time and a baseline number of rescue usage events of the user. The user's frequency of rescue usage events may include an average number of daily rescue usage events for the user for a predetermined period of time. The user's frequency of rescue usage events may include an absolute number of rescue usage events for the user for a predetermined period of time.

The training data may include an adherence ratio that indicates the user's adherence to the dosing schedule for the maintenance medicament type for each user of the plurality of users that is associated with at least one maintenance usage event, and/or a user's frequency of rescue usage events for each user of the plurality of users that is associated with at least one rescue usage event. The training data may include a number of usage events of a rescue medicament type for a user of the plurality of users in a last predetermined number of days. The training data may include a number of missed usage events of a maintenance medicament type for a user of the plurality of users over the last predetermined number of days. The training data may include a percent change in inhalation peak flow for a previous number of usage events for a user of the plurality of users compared to an average inhalation peak flow of the user. The training data may include a percent change in inhalation volume for a previous number of usage events for a user of the plurality of users compared to an average inhalation volume of the user.

The DHP may be configured to determine an environmental condition for each of the plurality of usage events using a respective time and geographic location associated with the usage event, where for example, training data may include the environmental condition for each of the plurality of usage events. The environmental condition may include any combination of temperature, humidity, outdoor air pollutants, particulate matter of 2.5 microns or smaller (PM2.5), particulate matter of 10 microns or smaller (PM10), ozone, nitrogen dioxide (NO₂), or sulfur dioxide (SO₂).

The unsupervised learning method may include a clustering method, such as a k-means or c-means clustering method. The supervised learning method may include gradient boosted decision trees and/or an XGBoost algorithm.

In some examples, the DHP may train the machine learning algorithm to perform pattern detection to determine the score (e.g., the future compliance score) for the user based on a comparison between a pattern associated with the user and patterns associated with the plurality of users. The pattern associated with the user may include the user's training data over a previous number of days, where each of the patterns associated with the plurality of users comprise training data associated with a respective user of the plurality of users over a period of time that equals the previous number of days.

The DHP may be configured to suggest an attribute (e.g., factor) that the user should improve upon to improve one or more of their scores. For example, the DHP may be configured to determine, using the trained machine learning algorithm, a significance factor for each of a plurality of attributes. The attributes may include the time of a usage event, one or more inhalation parameters of a usage event, the user's frequency of rescue usage events, and/or the adherence to the dosing schedule. The DHP may determine, using the trained machine learning algorithm, an attribute that the user should improve upon to improve a score (e.g., the compliance score and/or future compliance score) for the user based on the significance factor for each of the plurality of attributes. The DHP may cause the display device to generate a notification indicating the attribute that the user should improve upon. The notification may indicate that the user should take the maintenance medicament at a different time of day, increase the PIF of future usage events, or increase inhaled volume of future usage events.

The DHP may be configured to associate geolocation and/or one or more environmental factors with a usage event, for example, with an inhalation parameter(s) of a usage event. The DHP may be configured to determine a geographic location for the inhalation parameters of each of the plurality of usage events, train a machine learning algorithm using training data via a supervised or an unsupervised learning method, and determine, using the trained machine learning algorithm, a score for a user of the plurality of different users. The score may include the compliance score, the future compliance score, and/or the risk score for the user. The training data may include the time, the one or more inhalation parameters, and the geographic location associated with each of the plurality of usage events. Further, in some examples, a present geographic location of the user may be used to determine the score for the user.

In some examples, the DHP may determine an environmental condition for the inhalation parameter of each of the plurality of usage events based on the geographic location, and the training data may include the environmental condition for the inhalation parameter of each of the plurality of usage events. The environmental conditions may include any combination of temperature, humidity, outdoor air pollutant concentration, presence of particulate matter of 2.5 microns or smaller (PM2.5), presence of particulate matter of 10 microns or smaller (PM10), ozone concentration, nitrogen dioxide (NO2) concentration, and/or sulfur dioxide (SO2) concentration.

In some instances, the DHP may determine a point of interest for the inhalation parameter of each of the plurality of usage events based on the geographic location, and the training data may also include the point of interest for the inhalation parameter of each of the plurality of usage events. The point of interest for the inhalation parameter may include a park, a fuel station, a factory, a power plant, or a highway.

### BRIEF DESCRIPTION OF THE DRAWINGS

FIG. 1 is a block diagram of an example inhaler.

FIG. 2 is a graph of example flow rate versus time during use of an inhaler.

FIG. 3 is a diagram of an example system including example inhaler(s), user application(s), and digital health platform.

FIGS. 4A and 4B are diagrams of example systems for capturing data from a plurality of drug delivery devices, and aggregating and analyzing the data to provide insight into the use, efficacy, and efficiency of the drug delivery device and/or the therapy prescribed to patients suffering from certain diseases.

FIG. 5A illustrates an example procedure for enriching inhaler data.

FIG. 5B illustrates an example enrichment procedure for enriching inhaler data with data from an external source, such as an external weather source.

FIG. 5C illustrates an example procedure for determining a compliance score for a user.

FIG. 5D illustrates an example procedure for determining a future compliance score for a user.

FIG. 5E illustrates an example procedure for determining an attribute that should be improved by a user.

FIG. 5F illustrates an example procedure for determining a risk score for a user.

FIG. 5G illustrates an example procedure for determining a score for a user.

FIG. 6A shows a front perspective view of an exemplary inhaler.

FIG. 6B shows a top perspective view of the inhaler shown in FIG. 6A.

FIG. 7 shows a cross-sectional interior perspective view of the inhaler shown in FIG. 6A.

FIG. 8 provides an exploded perspective view of the example inhaler shown in FIG. 6A.

FIG. 9 provides an exploded perspective view of a top cap and electronics module of the inhaler shown in FIG. 6A.

FIG. 10 shows a graph of airflow rate through the example inhaler shown in FIG. 6A versus pressure.

### DETAILED DESCRIPTION

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the figures to indicate the same or similar parts.

The present disclosure describes devices, systems and methods for sensing, tracking processing, and/or presenting usage conditions and parameters associated with a drug delivery device, such as an inhaler. The devices, systems and methods are described in the context of a breath-actuated inhaler (e.g., also referred to herein as an inhaler) for delivering medication into a user's lungs. However, the described solutions are equally applicable to other drug delivery devices, such as an injector, a metered-dose inhaler, a nebulizer, a transdermal patch, or an implantable.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath.

The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate), fluticasone (propionate or furoate), mometasone (furoate), ciclesonide and dexamethasone (sodium). Parentheses indicate preferred salt or ester forms. Particular mention should be made of budesonide, beclomethasone and fluticasone, especially budesonide, beclomethasone dipropionate, fluticasone propionate and fluticasone furoate.

Different classes of bronchodilators target different receptors in the airways. Two commonly used classes are β2-agonists and anticholinergics.

β2-Adrenergic agonists (or “β2-agonists”) act upon the β2-adrenoceptors which induces smooth muscle relaxation, resulting in dilation of the bronchial passages. They tend to be categorized by duration of action. Examples of long-acting β2-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β2-agonists (SABA) are albuterol (sulfate) and terbutaline (sulfate). Particular mention should be made of formoterol, salmeterol, indacaterol and vilanterol, especially formoterol fumarate, salmeterol xinafoate, indacaterol maleate and vilanterol triphenylacetate.

Typically short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g. ICS-formoterol (fumarate), typically budesonide-formoterol (fumarate) or beclomethasone (dipropionate)-formoterol (fumarate). Thus, the rescue medicine is preferably a SABA or a rapid-acting LABA, more preferably albuterol (sulfate) or formoterol (fumarate), and most preferably albuterol (sulfate).

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), umeclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide). Particular mention should be made of tiotropium, aclidinium, umeclidinium and glycopyrronium, especially tiotropium bromide, aclidinium bromide, umeclidinium bromide and glycopyrronium bromide.

A number of approaches have been taken in preparing and formulating these medicaments for delivery by inhalation, such as via a dry powder inhaler (DPI), a pressurized metered dose inhaler (pMDI) or a nebulizer.

According to the GINA (Global Initiative for Asthma) Guidelines, a step-wise approach is taken to the treatment of asthma. At step 1, which represents a mild form of asthma, the patient is given an as needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localized to the airways, lung parenchyma and pulmonary vasculature. Phenotypically, these changes reduce the healthy ability of the lungs to absorb and expel gases.

Bronchitis is characterized by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localized hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β2-agonists and corticosteroids. The medicaments are often administered as a single therapy or as combination treatments.

Patients are categorized by the severity of their COPD using categories defined in the GOLD Guidelines (Global Initiative for Chronic Obstructive Lung Disease, Inc.). The categories are labelled A-D and the recommended first choice of treatment varies by category. Patient group A are recommended a short-acting muscarinic antagonist (SAMA) prn or a short-acting β2-agonist (SABA) prn. Patient group B are recommended a long-acting muscarinic antagonist (LAMA) or a long-acting β2-agonist (LABA). Patient group C are recommended an inhaled corticosteroid (ICS)+a LABA, or a LAMA. Patient group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e. a therapy going beyond their maintenance therapy. For example, the diagnosis of a clinical asthma exacerbation (CAE) may be based on the American Thoracic Society/European Respiratory Society statement (H. K. Reddel et al., Am J Respir Crit Care Med. 2009, 180(1), 59-99). It includes both a “severe CAE” and a “moderate CAE.” A severe CAE may be defined as a CAE that involves worsening asthma that requires oral steroid (prednisone or equivalent) for at least three days and hospitalization. A moderate CAE may be defined as a CAE that requires oral steroid (prednisone or equivalent) for at least three days or hospitalization.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation are repeated doses of SABA, oral corticosteroids and/or antibiotics. A severe exacerbation adds controlled flow oxygen and/or respiratory support (both of which require hospitalization). An exacerbation within the meaning of the present disclosure includes both moderate and severe exacerbations.

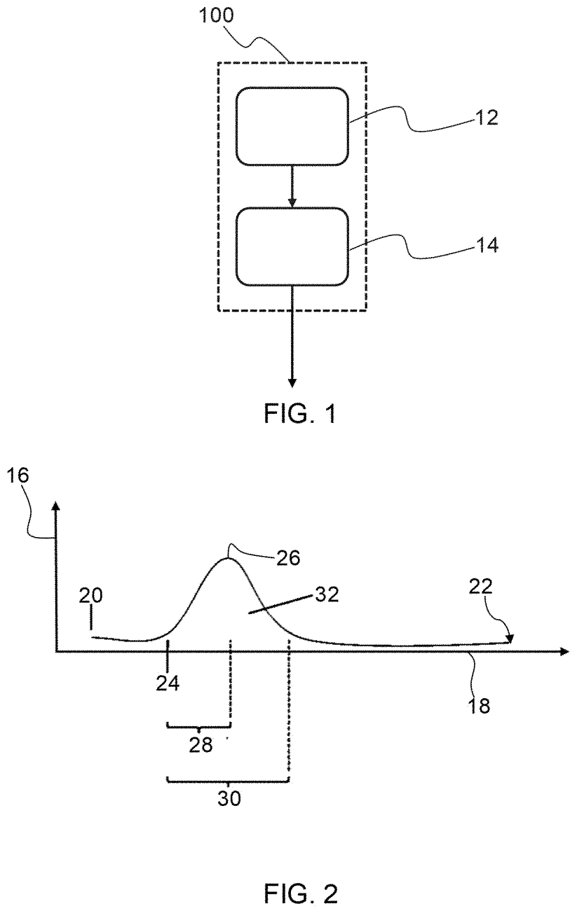

FIG. 1 shows a block diagram of an inhaler 100 according to an example. The inhaler 100 comprises a use determination system 12 that detects usage events. For example, a usage event may include usage of the inhaler 100, such as a movement of a mouthpiece cover of the inhaler 100 from a closed to an open position, the priming of a dose of medicament in the inhaler, such as the shaking of the inhaler 100 (e.g., when the inhaler 100 is a MDI), the actuation of a switch of the inhaler or a twist of a cap of the inhaler that advances a blister strip or prepares a dose of medicament, and/or the recording of an inhalation through the inhaler 100 by a user (e.g., an inhalation event). Each usage event may include one or more usage parameters that are determined by the use determination system 12. Usage parameters may include any number of parameters indicative of a usage event at the inhaler 100 by a subject. For example, usage parameters may include the duration of the shaking of the inhaler 100, the orientation of the inhaler 100 during an inhalation, the temperature or humidity of the inhaler during an inhalation, and/or an inhalation parameter (e.g., flow rate) that is measured during an inhalation event. The usage parameters may be measured by the use determination system 12, for example, in response to detecting a given usage event. Specifically, and as further described herein, the use determine system 10 may measure one or more inhalation specific usage parameters in response to detecting an inhalation event. The usage parameters measured by the use determination system 12 for an inhalation event, also referred to herein as inhalation parameter(s) (e.g., as the relevant device is an inhaler), may include one or more of: a flow rate, a peak inspiratory/inhalation flow (PIF), an inhalation volume, a time to peak inhalation flow, and/or an inhalation duration.

The usage parameter(s) is received by a transmission module 14, as represented in FIG. 1 by the arrow between the block representing the use determination system 12 and the block representing the transmission module 14. The transmission module 14 encrypts data based on the value(s) of the usage parameter, and transmits the encrypted data, as represented in FIG. 1 by the arrow pointing away from the transmission module 14 block. The transmission of the encrypted data by the transmission module 14 may, for example, be wireless, as previously described.

The transmission module 14 is configured to transmit the respective encrypted data wirelessly. A transceiver configured to implement any suitable wireless transmission protocol may be included in the transmission module 14, such as via Wi-Fi, Wi-MAX, Bluetooth®, Bluetooth® Smart, ZigBee, near field communication (NFC), cellular communication, television white space (TVWS) communication, or any combination thereof.

Although examples described herein may refer to a transceiver, the transceiver may be configured to transmit, but not receive, data (e.g., a transmitter but not a receiver). The transceiver may include one or more semiconductor chips, integrated circuits, and/or the like configured to implement the logic and procedures of the communication protocol. The transceiver may include radio frequency (RF) hardware such as amplifier(s), oscillator(s), modulator circuit(s), antenna(s), antenna tuner(s), and/or the like in order to transmit signals wirelessly using the communication protocol. The RF hardware may be implemented in whole or in part on the semiconductor chip(s), integrated circuit(s), and/or the like configured to implement the logic and procedures of the communication protocol.

In some examples, the transmission module 14 is configured to transmit and/or receive data via Bluetooth®. Bluetooth® may be used because the relatively low energy associated with transmitting and receiving may preserve the battery life of the respective inhaler. Moreover, no internet connection need be established in order for the respective encrypted data to be transmitted.

The use determination system 12 may comprise a suitable processor and memory configured to perform the functions described herein for the processing module. For example, the processor may be a general purpose processor programmed with computer executable instructions for implementing the functions of the use determination system 12. The processor may be implemented using a microprocessor or microcontroller configured to perform the functions of the use determination system 12. The processor may be implemented using an embedded processor or digital signal processor configured to perform the functions of the use determination system 12.

As described herein, a usage parameter may, for example, comprise an indication of a use of the respective inhaler. In a relatively simple implementation, the at least one value may indicate whether a usage event has been detected by the use determination system 12 and comprise “TRUE” when use of, for example an inhalation using, the respective inhaler has been determined, or “FALSE” when no such use of the respective inhaler is determined.

The use determination system 12 may also include one or more components or sensors to determine usage parameter(s) of inhaler 100. For example, a usage parameter may be one or more measurements performed by the use determination system (e.g., or a respective component or sensor of the use determination system 12) of a count of the number of uses inhaler 100, a measure of airflow of inhaler 100, and/or other measurements indicating the usage of the medicament of inhaler 100. The use determination system 12 may include one or more of a switch configured to detect usage of inhaler 100, one or more sensors configured to detect use of inhaler 100, one or more buttons configured to be depressed upon use of inhaler 100, and/or the like.

For example, the use determination system 12 may, for instance, comprise a mechanical switch configured to be actuated prior to, during, or after use of the respective inhaler. The mechanical switch may indicate that a dose of medicament has been primed and is ready for inhalation (e.g., such as by metering a dose from a hopper, advancing and/or opening a blister pack, breaking open a pill comprising medicament, etc.). In a non-limiting example, the inhaler 100 comprises a medicament reservoir (not visible in FIG. 1), and a dose metering assembly (not visible in FIG. 1) configured to meter a dose of the rescue medicament from the reservoir. The use determination system 12 may be configured to register the metering of the dose by the dose metering assembly, each metering being thereby indicative of the inhalation performed by the subject using the inhaler 100. Accordingly, the inhaler 100 may be configured to monitor the number of inhalations of the medicament, since the dose should be metered via the dose metering assembly before being inhaled by the subject. One non-limiting example of the dose metering assembly will be explained in greater detail with reference to FIGS. 12-16.

Alternatively or additionally, the use determination system 12 may register each inhalation in different manners and/or based on additional or alternative feedback. For example, the use determination system 12 may be configured to register an inhalation by the subject when the feedback from a suitable sensor (not visible in FIG. 1) indicates that an inhalation by the subject has occurred, for example when a pressure measurement or flow rate exceeds a predefined threshold associated with a successful inhalation.

A sensor, such as a pressure sensor, an acoustic sensor, or a flow sensor, may, for example, be included in the use determination system 12 in order to register each inhalation. Such a sensor may be an alternative or in addition to the abovementioned mechanical switch. When a pressure or acoustic sensor is included in the use determination system 12, the pressure sensor may, for instance, be used to confirm that, or assess the degree to which, a dose metered via the dose metering assembly is inhaled by the subject, as will be described in greater detail with reference to FIGS. 2 and 12-16.

More generally, the use determination system 12 may comprise a sensor for detecting a parameter relating to airflow during inhalation of the respective medicament performed by the subject. In other words, the usage parameter may comprise a parameter relating to airflow during inhalation of the medicament. The at least one value may thus, for example, comprise a numerical value relating to the detected inhalation parameter.

In some examples, the use determination system 12 may receive pressure measurements from a pressure sensor, and may calculate an inhalation parameter, such as flow, based on the pressure measurements. The inhalation parameter (e.g., inhalation parameter) may be, for example, at least one of a PIF, an inhalation volume, a time to peak inhalation flow, and an inhalation duration. As detailed further herein, the use determination system 12 may use the inhalation parameter to classify a respective usage event and/or inhalation event (e.g., a good inhalation event, a fair inhalation event, a low/no inhalation event, an exhalation event, a possible air vent block event, etc.). In such examples, the at least one value comprises a numerical value for the PIF, the inhalation volume, the time to peak inhalation flow, and/or the inhalation duration. Further, in some examples, the user determination system 12 may determine and store a flow profile (e.g., a series of consecutive pressure measurements) associated with a usage event. As described herein, these inhalation parameters (e.g., also referred to herein as inhalation parameters) may be used to assess the user's compliance, which may provide valuable insight into the state of the user's respiratory disease (e.g., the likelihood that the patient is to experience an exacerbation event) and/or the patient's sensitivity to current or changing environmental conditions (e.g., heat, air quality, humidity, etc.).

A pressure sensor may be particularly suitable for measuring the parameter, since the airflow during inhalation by the subject may be monitored by measuring the associated pressure changes. The pressure sensor may be located within or placed in fluid communication with a flow pathway through which air and the medicament is drawn by the subject during inhalation. An example of a pressure sensor that is in fluid communication with a flow pathway of the inhaler is described with reference to FIGS. 12-16. Alternative ways of measuring the parameter, such as via a suitable flow sensor, can also be used.

An inhalation may be associated with a decrease in the pressure in the airflow channel of the inhaler relative to when no inhalation is taking place. The point at which the pressure change is at its greatest may correspond to the peak inhalation flow. The pressure sensor may detect this point in the inhalation.

The pressure change associated with an inhalation may alternatively or additionally be used to determine an inhalation volume. This may be achieved by, for example, using the pressure change during the inhalation measured by the pressure sensor to first determine the flow rate over the time of the inhalation, from which the total inhaled volume may be derived.

The pressure change associated with an inhalation may alternatively or additionally be used to determine an inhalation duration. The time may be recorded, for example, from the first decrease in pressure measured by the pressure sensor, coinciding with the start of the inhalation, to the pressure returning to a pressure corresponding to no inhalation taking place.

The inhalation parameter may alternatively or additionally include the time to peak inhalation flow. This time to peak inhalation flow parameter may be recorded, for example, from the first decrease in pressure measured by the pressure sensor, coinciding with the start of the inhalation, to the pressure reaching a minimum value corresponding to peak flow.

Further, in some examples, the inhalation parameter may be a flow profile that includes a plurality of measurements from the pressure sensor taken over time (which includes one or more of a peak inhalation flow, an inhalation volume, a time to peak inhalation flow, and/or an inhalation duration). For example, the use determination system 12 may turn on the pressure sensor and/or begin receiving measurements from the pressure sensor in response to switching from a low power state, such as an off state or a sleep state, to an on state. The use determination system 12 may calculate a flow profile using the received pressure measurements. The use determination system 12 may compare the measurements received from the pressure sensor (e.g., the flow profile) to one or more thresholds and determine whether the measurements are indicative of an inhalation by the user. For instance, the use determination system 12 may determine that the measurements are indicative of an inhalation by the user when a pressure measurement exceeds a predetermined value or is within a predetermine range that is indicative of peak inhalation by a user, the pressure measurements indicate a slope that is indicative of inhalation, the pressure measurements indicate an inhalation duration that is within a predetermined time range, and/or the like. After determining that the measurements are indicative of an inhalation by the user, the use determination system 12 may store a plurality of the measurements received from the pressure sensor as a flow profile associated with the inhalation event.

FIG. 2 shows a graph of flow rate 16 versus time 18 during use of an inhaler 100 according to a non-limiting example. The use determination system 12 in this example comprises a mechanically operated switch in the form of a switch which is actuated when a mouthpiece cover of the inhaler 100 is opened. The mouthpiece cover is opened at point 20 on the graph. In this example, the use determination system 12 further comprises a pressure sensor.

When the mouthpiece cover is opened, the use determination system 12 is woken out of an energy saving sleep mode, and a new inhalation event is registered. The inhalation event is also assigned an open time corresponding to how much time, for example milliseconds, elapses since the inhaler 100 wakes from the sleep mode. Point 22 corresponds to the cap closing or 60 seconds having elapsed since point 20. At point 22, detection ceases. Although described as occurring when the mouthpiece cover is opened, in other examples, the use determination system 12 may switch power states and record a new inhalation event based on the inhaler being primed for use, such as any combination of the actuation of a switch that advances a blister strip, the twist of a cap that breaks a capsule of medication, the detection of the inhaler being shaken for a predetermined amount of time, etc.

Once the mouthpiece cover is open, the use determination system 12 looks for a change in the air pressure, as detected using the pressure sensor. In examples where the pressure sensor is an absolute pressure sensor, the use determination system 12 may analyze a rolling number of consecutive pressure measurements to determine a tare value, or dynamic zero value, to calibrate the pressure sensor to the atmospheric pressure, and then use the tare value to determine a change in air pressure that may be indicative of the start of an inhalation event (e.g., a slope that exceeds a threshold indicative of inhalation).

The start of the air pressure change is registered as the inhale event time 24. The point at which the air pressure change is greatest corresponds to the peak inhalation flow 26. The use determination system 12 records the peak inhalation flow 26 as a flow of air, measured in units of 100 mL per minute, which flow of air is transformed from the air pressure change. Thus, in this example, the at least one value comprises a value of the peak inhalation flow in units of 100 mL per minute. The corresponding usage information provided via a user interface may, for example, express this peak inhalation flow using the same units or in liters per minute.

The time to peak inhalation flow 28 corresponds to the time taken in milliseconds for the peak inhalation flow 26 to be reached. The inhalation duration 30 corresponds to the duration of the entire inhalation in milliseconds. The area under the graph 32 corresponds to the inhalation volume in milliliters. Further, the collection of measurements from 20 to 22 (or a subset of these measurements) may be stored by the use determination system as a flow profile associated with the usage or inhalation event.

The usage information provided via the user interface may, additionally or alternatively to providing the inhalation parameter(s) as numerical values, provide a classification of one or more (or each) inhalation event(s). For example, if the peak inhalation flow is between 0 and 30 liters per minute, the inhalation event is classified as “low inhalation” (less than or equal to 30 liters per minute) or as “no inhalation”, if no inhalation is detected within 60 seconds of the mouthpiece cover being open. If the peak inhalation flow is greater than 45 and less than or equal to 200 liters per minute, the inhalation event is classified as a “good inhalation”. If the peak inhalation flow is greater than 30 and less than or equal to 45 liters per minute, the inhalation event is classified as “fair”. If the peak inhalation flow is above 200 liters per minute, the inhalation event is classified as a “possible air vent block”. The inhalation event may be classified as an “exhalation”. For instance, a negative change in pressure may correspond to an inhalation, while a positive change in pressure may correspond to an exhalation. In instances where the sensor is a flow sensor, a pressure change airflow being detected in the opposite direction to that expected for inhalation using the inhaler 100 may be indicative of an exhalation. The classification of inhalation parameter(s) as a respective inhalation event (e.g., a good inhalation event, a fair inhalation event, a low/no inhalation event, an exhalation event, a possible air vent block event, etc.) may be based on or result from calculations performed at an End-User or Patient facing processing module of a user device, as described herein.

In a non-limiting example, the inhaler 100 is configured such that, for a normal inhalation, the medicament is dispensed approximately 0.5 seconds following the start of the inhalation. A subject's inhalation only reaching peak inhalation flow after the 0.5 seconds have elapsed, such as after approximately 1.5 seconds, may be partially indicative of the subject having difficulty in controlling their respiratory disease. Such a time to reach peak inhalation flow may, for example, be indicative of the subject facing an impending exacerbation.

More generally, the use determination system 12 may employ respective sensors (e.g. respective pressure sensors) for registering an inhalation/use of the inhaler and detecting the inhalation parameter, or a common sensor (e.g. a common pressure sensor) which is configured to fulfil both inhalation/use registering and inhalation parameter detecting functions.

Any suitable sensor may be included in the use determination system 12, such as one or more pressure sensors, temperature sensors, humidity sensors, orientation sensors, acoustic sensors, and/or optical sensors. The pressure sensor(s) may include a barometric pressure sensor (e.g. an atmospheric pressure sensor) or a differential pressure sensor. The sensors may employ microelectromechanical systems (MEMS) and/or nanoelectromechanical systems (NEMS) technology.

In a non-limiting example, the use determination system 12 comprises a differential pressure sensor. The differential pressure sensor may, for instance, comprise a dual port type sensor for measuring a pressure difference across a section of the air passage through which the subject inhales. A single port gauge type sensor may alternatively be used. The latter operates by measuring the difference in pressure in the air passage during inhalation and when there is no flow. The difference in the readings corresponds to the pressure drop associated with inhalation.

In another non-limiting example, the use determination system 12 includes an acoustic sensor. The acoustic sensor in this example is configured to sense a noise generated when the subject inhales through the inhaler 100. The acoustic sensor may include, for example, a microphone. The inhaler 100 may, for instance, comprise a capsule which is arranged to spin when the subject inhales though the device; the spinning of the capsule generating the noise for detection by the acoustic sensor. The spinning of the capsule may thus provide a suitably interpretable noise, e.g. rattle, for deriving use and/or inhalation parameter data.

An algorithm may, for example, be used to interpret the acoustic data in order to determine use data and/or the parameter relating to airflow during the inhalation. For instance, an algorithm as described by P. Colthorpe et al., “Adding Electronics to the Breezhaler: Satisfying the Needs of Patients and Regulators”, Respiratory Drug Delivery 2018, 1, 71-80 may be used. Once the generated sound is detected, the algorithm may process the raw acoustic data to generate the use and/or inhalation parameter data.

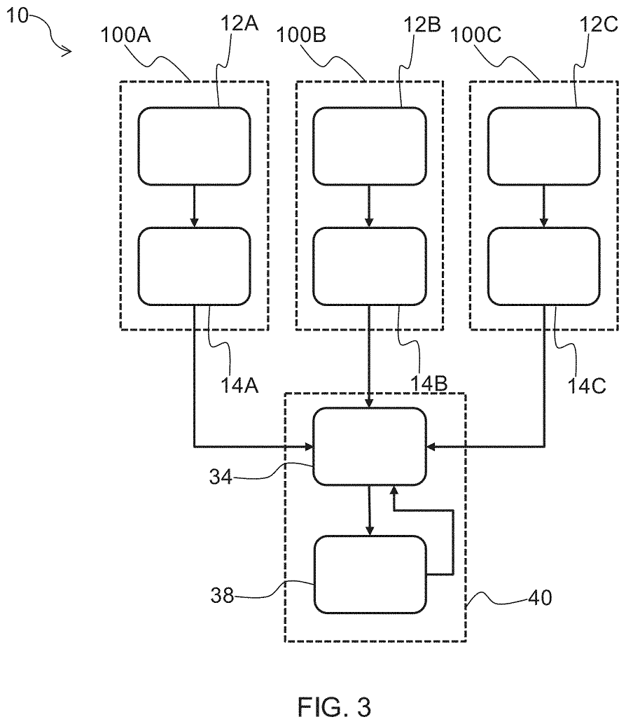

FIG. 3 shows a block diagram of a system 10 according to a non-limiting example. The system 10 may, for example, be alternatively termed “an inhaler assembly”. For example, the system 10 may be associated with a specific user or patient.

As shown in FIG. 3, the system 10 comprises a first inhaler 100A comprising a first use determination system 12A and a first transmission module 14A. The system 10 further comprises a second inhaler 100B comprising a second use determination system 12B and a second transmission module 14B. The first inhaler 100A delivers a first medicament, and the second inhaler 100B delivers a second medicament which in some examples is different from the first medicament, as previously described.

In some examples, such as the system 10 depicted in FIG. 3, the system 10 may further include a third inhaler 100C comprising a third use determination system 12C, and a third transmission module 14C. The third inhaler 100C delivers a third medicament which is different from the first and second medicaments. In other examples, no third inhaler 100C is included in the system 10, or a fourth, fifth, etc. inhaler (not visible) is included in addition to the first inhaler 100A, the second inhaler 100B, and the third inhaler 100C. Alternatively or additionally, the system 10 includes a plurality of first inhalers 100A, a plurality of second inhalers 100B, and so on, as previously described.

The system 10 comprises a processing module 34 which is configured to receive the respective encrypted data transmitted from each of the transmission modules 14A, 14B, 14C, as represented in FIG. 3 by the arrows between each of the blocks corresponding to the transmission modules 14A, 14B, 14C and the block corresponding to the processing module 34. The first, second, and/or third encrypted data may be transmitted wirelessly to the processing module 34, as previously described. The processing module 34 may thus comprise a suitable receiver or transceiver for receiving the encrypted data. The receiver or transceiver of processing module 34 may be configured to implement the same communication protocols as transmission modules 14A, 14B, 14C and may thus include similar communication hardware and software as transmission modules 14A, 14B, 14C as described herein (not shown in FIG. 3).

Although examples described herein may refer to a transceiver, the transceiver may be configured to transmit, but not receive, data (e.g., a transmitter but not a receiver). The transceiver may include one or more semiconductor chips, integrated circuits, and/or the like configured to implement the logic and procedures of the communication protocol. The transceiver may include radio frequency (RF) hardware such as amplifier(s), oscillator(s), modulator circuit(s), antenna(s), antenna tuner(s), and/or the like in order to transmit signals wirelessly using the communication protocol. The RF hardware may be implemented in whole or in part on the semiconductor chip(s), integrated circuit(s), and/or the like configured to implement the logic and procedures of the communication protocol.

The processing module 34 may comprise a suitable processor and memory configured to perform the functions described herein for the processing module. For example, the processor may be a general purpose processor programmed with computer executable instructions for implementing the functions of the processing module. The processor may be implemented using a microprocessor or microcontroller configured to perform the functions of the processing module. The processor may be implemented using an embedded processor or digital signal processor configured to perform the functions of the processing module. In an example, the processor may be implemented on a smartphone or other consumer electronic device that is capable of communicating with transmission modules 14A, 14B, 14C and performing the functions of the processing module 34 as described herein. For example, the processing module may be implemented on a user device, such as a smart phone or consumer electronic device, that includes an application (e.g., a mobile application) that causes the processor of the user device to perform the functions of the processing module 34 as described herein.

The processing module 34 distinguishes between the first encrypted data, the second encrypted data, and the third encrypted data, for example by using respective identifiers, as previously described.

The processing module 34 determines first usage information relating to the first medicament based on the distinguished first encrypted data. The first usage information may comprise a registered use of, or inhalation performed using, the first inhaler 100A, and/or a parameter relating to airflow during such an inhalation using the first inhaler 100A, as previously described. The usage information may include the usage parameter or information derived from the usage parameter. And in some examples, usage parameters may be inhalation parameters (e.g., inhalation flow, peak inhalation flow, etc.) and an example usage event may be an inhalation event (e.g., an inhalation of an inhaler by a user).

Similarly, the processing module 34 determines second and third usage information relating to the second and third medicaments respectively, based on the distinguished second and third encrypted data.

The system 10 further comprises a user interface 38. The processing module 34 is configured to control the user interface 38 to communicate the first, second, and/or third usage information. The arrow pointing from the block representing the processing module 34 to the block representing the user interface 38 is intended to represent the control signal(s) which causes or cause the user interface to communicate, for example display, the respective usage information. In this respect, the user interface 38 may comprise any suitable display, screen, for example touchscreen, etc. which is capable of displaying the respective usage information. Alternatively or additionally, the respective usage information may be provided by the user interface 38 via an audio message. In such an example, the user interface 38 comprises a suitable loudspeaker for delivering the audio message. Numerous ways of communicating the respective usage information can be used.

The system 10 thus enables the subject to be informed of their usage of the respective medicaments, which may be administered according to a treatment regimen and/or an administration protocol specific to the respective medicament, as previously described.

Whilst the transmission modules 14A, 14B, 14C are each shown in FIG. 3 as transmitting (encrypted) data to the processing module 34, this is not intended to exclude the respective inhalers 100A, 100B, 100C, or a component module thereof, receiving data transmitted from the processing module 34.

In a non-limiting example, a clock module (not visible in the Figures) is included in each of the respective inhalers 100A, 100B, 100C for assigning a time, for example a time stamp, to the usage parameter of the respective inhaler 100A, 100B, 100C. In this example, the processing module 34 is configured to synchronize the clock modules of the respective inhalers 100A, 100B, 100C. Such synchronization may, for instance, provide a point of reference which enables the relative timing of use of the respective inhalers 100A, 100B, 100C to be determined, which may have clinical relevance, as previously described. The assigned time, for example time stamp, may, for instance, be included in the usage information for the respective medicaments communicated, e.g. displayed, by the user interface 38.

Whilst not shown in FIG. 3, the processing module 34 may, in some examples, comprise a further clock module. The clock modules of each of the respective inhalers 100A, 100B, 100C may thus be synchronized according to the time provided by the further clock module. The further clock module may, for instance, receive the time of the time zone in which the processing module 34 is situated. This may cause the respective inhalers 100A, 100B, 100C to be synchronized according to the time in which the subject and their respective inhalers 100A, 100B, 100C are located, which may provide further information of clinical relevance, as previously described.

In an embodiment, the processing module 34 is at least partly included in a first processing module included in the user device 40. By implementing as much processing as possible of the usage information from the respective inhalers 100A, 100B, 100C in the first processing module of the user device 40, battery life in the respective inhalers 100A, 100B, 100C may be advantageously saved. The user device 40 may be, for example, at least one selected from a personal computer, a tablet computer, or a smart phone. And in some examples, the processing module 34 may include (e.g., be) a mobile application residing on the user device 40.

Alternatively or additionally, the user interface 38 may be at least partly defined by a first user interface of the user device 40. The first user interface of the user device 40 may, for instance, comprise, or be defined by, the touchscreen of a smart phone 40.

In other non-limiting examples, the processing module is not included in a user device. The processing module (or at least part of the processing module 34) may, for example, be provided in a server, e.g. a remote server such as the DHP.

Provided herein is a system for capturing data from a plurality of drug delivery devices (e.g., via a mobile device in communication with the drug delivery device), such as the inhaler 100, and aggregating and analyzing the data to provide insight into the use, efficacy, and efficiency of the drug delivery device and/or the therapy prescribed to patients suffering from certain diseases. As further detailed in FIG. 3, the system comprises a plurality of inhalers, one or more End-User or Patient facing processing module(s), a central processing module (e.g., referred to herein as a digital health platform (DHP)), and one or more Care Provider facing processing module(s). The End-User or Patient facing processing module(s) may be in communication with and/or receive data from (e.g., paired with) at least one of the plurality of inhalers, and may forward the data (e.g. or a subset thereof) to the central processing module. The central processing module, or DHP, may receive, aggregate, and perform analysis on the data received from the End-User or Patient facing processing module(s). In addition, the central processing module may provide (e.g., via an interface) the aggregate data and the associated analytics to both the End-User or Patient facing processing module(s) and the Care Provider facing processing module(s).

Also provided is a computer program comprising computer program code which is adapted, when the computer program is run on a computer, to implement any of the above-described methods. In an embodiment, the computer program takes the form of an app, for example an app for a user device 40, such as a mobile device, e.g. tablet computer or a smart phone.

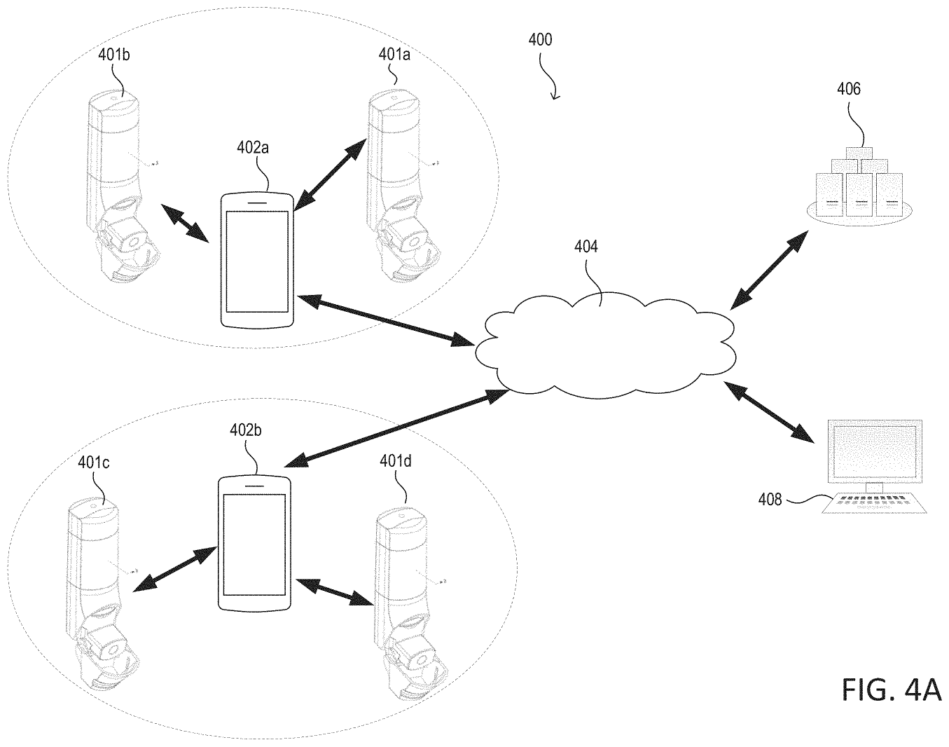

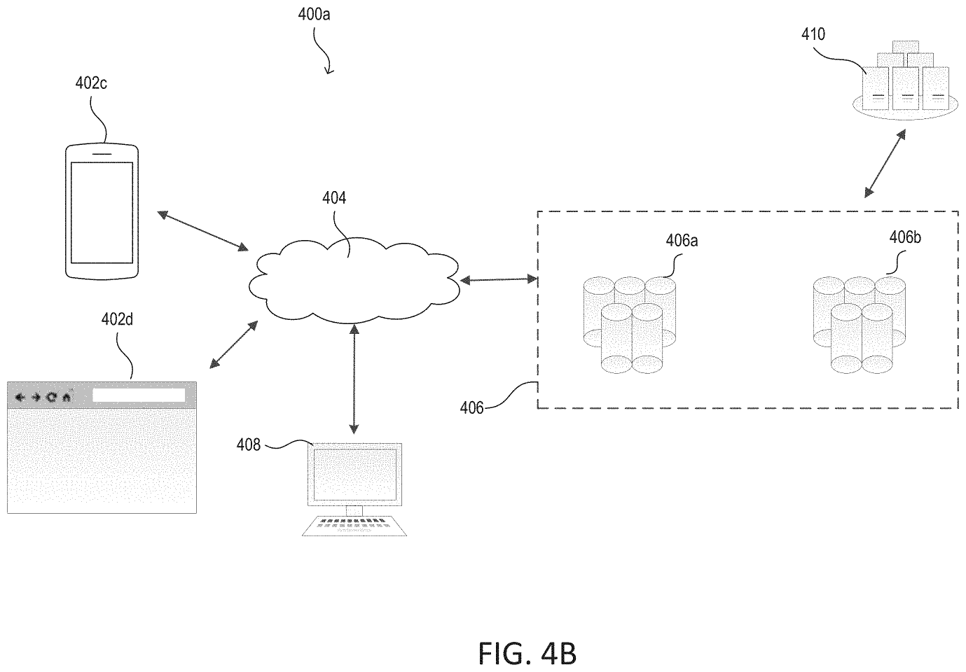

FIG. 4A is a diagram of an example system 400 for capturing data from a plurality of drug delivery devices (e.g., via a mobile device in communication with the drug delivery device), such as the inhaler 100, and aggregating and analyzing the data to provide insight into the use, efficacy, and efficiency of the drug delivery device and/or the therapy prescribed to patients suffering from certain diseases. As illustrated in FIG. 4A, the system 400 include inhalers 401a-d (e.g., each of which may be an example of the inhaler 100), user devices 402a, 402b that comprises an End-User or Patient facing processing module (e.g., a user device comprising a mobile application), a public and/or private network 404 (e.g., the Internet, a cloud network, etc.), and a computer or server 408 that comprises (e.g., runs or otherwise interacts with) a Care Provider facing processing module (e.g., also referred to herein as a Dashboard application) associated with a health care provider, such as a hospital or hospital system, a health system, a medical group, a physician, a clinic, and/or a pharmaceutical company. The system 400 also includes a digital health platform (DHP) 406 (e.g., a central processing module) that resides on one or more servers, and may include computer software configured to perform the functions described in relation to the DHP 406.

As illustrated in FIG. 4A, the system 400 comprises inhalers 401a-d, each of which may be configured to deliver medicament to a user (e.g., a patient). The inhalers that are configure to deliver medicated to a given user may, along with at least one user device, form an inhaler assembly, as described herein, which may be indicated by the dashed circles. For example, referring to FIG. 4A, inhalers 401a, 401b, and user device 402a may form a first inhaler assembly that is configured to deliver medicaments to a first user. Similarly, inhalers 401c, 401d, and user device 402b may form a second inhaler assembly that is configured to deliver medicaments to a second user. As described earlier with respect to FIGS. 1-3, each of the inhalers 401a-d comprise a use determination system configured to determine usage parameters relating to use of the respective inhaler. Each of the inhalers 401a-d also comprise a transmission module configured to encrypt data based on the determined usage parameters, and then transmit the encrypted data. The medicament delivered by each of the inhalers 401a-d may be different. For example, the medicament delivered by inhaler 401a may differ from the medicament delivered by inhaler 401b. Accordingly, the usage parameters determined for each of the inhalers 401a-d may vary based on the medicament delivered by a respective inhaler.

In a non-limiting example, the medicaments delivered by inhalers 401a, 401c may include a rescue medicament for use by the subject as needed, and the medicaments delivered by inhalers 401b, 401d may include a maintenance medicament that is used by the subject according to a predetermined dosing schedule.

The rescue medicament is typically a SABA or a rapid-onset LABA, such as formoterol (fumarate). The rescue medicament may also be in the form of a combination product, e.g. ICS-formoterol (fumarate), typically budesonide-formoterol (fumarate) or beclomethasone (dipropionate)-formoterol (fumarate). Such an approach is termed “MART” (maintenance and rescue therapy).

In some non-limiting examples, an inhaler assembly may include multiple inhalers that deliver the same medicament. Such a plurality of inhalers delivering the same medicament may be particularly advantageous when, for example, the medicament is a rescue medicament. In such an example, the subject may place the multiple inhalers in various different locations, such as on a nightstand, in a gym bag, in a vehicle, and so on, so that the rescue medicament is readily available if needed.

In a non-limiting example, the rescue medicament is albuterol (sulfate), and the maintenance medicament is fluticasone (propionate or furoate), or salmeterol (xinafoate) combined with fluticasone (propionate or furoate).

More generally, the rescue medicament and the maintenance medicament (or any further medicaments included in any further inhalers included in the system) may comprise any suitable active pharmaceutical ingredient. Thus, any class of medication may be housed within and delivered by the inhalers 401a-401d. That is, the system 400 permits consolidated handling and communicating of usage information irrespective of the particular medications that are delivered by the inhalers.

As described herein, the inhalers 401a-d may each comprise a use determination system. The use determination system is configured to detect usage events (e.g., inhalation events) of the respective inhalers 401a-d. A usage event includes a detected usage of the inhaler 100, such as a movement of a mouthpiece cover of the inhaler 100 from a closed to an open position, the priming of a dose of medicament in the inhaler, such as the shaking of the inhaler 100 (e.g., when the inhaler 100 is a MDI), or the actuation of a switch of the inhaler or a twist of a cap of the inhaler that advances a blister strip or prepares a dose of medicament, and/or the recording of an inhalation through the inhaler 100 by a user (e.g., an inhalation event). Each usage event may include one or more usage parameters that are determined by the use determination system. Usage parameters may include any number of parameters indicative of a usage event at the inhaler 100 by a subject. For example, usage parameters may include the duration of the shaking of the inhaler 100, the orientation of the inhaler 100 during an inhalation, the temperature or humidity of the inhaler during an inhalation, and/or an inhalation parameter (e.g., flow rate) that is measured during an inhalation event. In some examples, a usage parameter may be an inhalation parameter, such as a peak inspiratory/inhalation flow (PIF), an inhalation volume, a time to peak inhalation flow, and/or an inhalation duration.

The use determination system may associate a relative timestamp to each the usage events. The use determination system may determine the relative timestamps using a respective clock module of the inhalers 401a-d. All of the information captured by a respective inhaler in association with a detected usage event may also be referred to herein as inhaler data. And, as described herein, the inhaler data may be transmitted to an End-User or Patient facing processing module running on a user device paired with the inhaler.

Each of the inhalers 401a-d may comprise a transmission module configured to encrypt data based on the determined usage events, and then transmit the encrypted data. For example, the transmission modules may each include an encryption device capable of encrypting the respective inhaler's data. For example, the encryption device may be implemented using hardware such a digital signal processor (DSP), a microcontroller, a processor, and/or the like. The encryption device may be incorporated into other portions of the transmission modules, such as a transceiver used to transmit the encrypted data. Examples of different types of transceivers are described in more detail below. Further, it should be appreciated that in some examples, the system 400 may also include smart add-on devices that include, for example, processing modules and/or a transmission modules (e.g., a processor, memory, and transmitter) that are configured to attach to otherwise “dumb” inhalers instead of, or in addition to, including the inhalers that are described herein.

Inhalers 401a-d may, via their respective transmission modules, transmit their data to a paired user device (e.g., a user device in the inhaler assembly), for example, via the End-User or Patient facing processing module running on the user device. A user device may pair with an inhaler using, for example, the End-User or Patient facing processing module running on the user device. For example, the user device may pair with a respective inhaler that is in close proximity to the user device (e.g., which, again, is illustrated by the dashed circles in FIG. 4A), such that the transmission modules on the inhaler and the user device can communicate with (e.g., send information to and from) each other. Referring again to FIG. 4A, for example, inhalers 401a, 401b may be paired with user device 402a to form the first inhaler assembly, and transmit their respective data to user device 402a via the End-User or Patient facing processing module running on user device 402a. Similarly, inhalers 401c, 401d may be paired with user device 402b to form the second inhaler assembly and transmit their respective data to user device 402b via the End-User or Patient facing processing module running on user device 402b. After pairing with a given user device, inhalers 401a-d may transmit data periodically or in response to detecting, via the respective use determination system, a usage event (e.g., inhalation event, opening of the inhaler cap, medicament priming, etc.).

The inhalers 401a-d may pair with a user device using, for example, an identifier, such as a barcode or a QR code, printed on the inhaler or its packaging. As further described herein, the inhaler's identifier may indicate certain information associated with the inhaler, such as the respective medicament delivered by the inhaler and the dose strength of the medicament. In turn the information associated with the inhaler may be used by the End-User or Patient facing processing module (e.g., or another processing module in the system 400, such as the DHP 406) running on a user device to issue notifications if the dose strength of the medicament in the inhaler as denoted by the identifier is different from the dose strength of the medicament in the existing inhaler. Such a notification may, for example, comprise a notification informing the user that the dose strength of an inhaler is different from that of the existing inhaler and/or a notification to request that the subject discards the existing inhaler. In this manner, the system 400 may assist the subject to adjust to a prescription change, for example, via the End-User or Patient facing processing module running on a user device (e.g., or another processing module in the system 400, such as the DHP 406).

Also, or alternatively, the information associated with an inhaler may be used to issue notifications, for example, via the End-User or Patient face processing module controlling the user interface of a user device, according to the user selection to remind the subject to use the inhaler according to a treatment regimen relating to administering of the maintenance medicament.

As illustrated in FIG. 4, the system 400 comprises user devices 402a, 402b, which may be a smart phone (e.g., an iPhone® smart phone, an Android® smart phone, or a Blackberry® smart phone), a personal computer, a laptop, a wireless-capable media device (e.g., MP3 player, gaming device, television, a media streaming devices (e.g., the Amazon Fire TV, Nexus Player, etc.), a tablet device (e.g., an iPad® hand-held computing device), a Wi-Fi or wireless-communication-capable television, a hub device or smart speaker (e.g., Amazon Echo, Google Home, etc.), or any other suitable Internet-Protocol-enabled device.

The user devices 402a, 402b (e.g., the End-User or Patient facing processing module) may include a transmission module, as described herein, and as such may be configured to transmit and/or receive RF signals via a Wi-Fi communication link, a Wi-MAX communications link, a Bluetooth® or Bluetooth Smart communications link, a near field communication (NFC) link, a cellular communications link, a television white space (TVWS) communication link, or any combination thereof. As further described herein, the user devices 402a, 402b may transfer data (e.g., through the public and/or private network 404) to the DHP 406 using, for example, a communication interface published by the DHP, such as a dedicated API. For example, the user devices 402a, 402b may send usage data relating to one or more paired inhalers to the DHP 406. That is, user device 402a may send usage data relating to inhalers 401a, 401b to the DHP 406, while user device 402b may send usage data relating to inhalers 401c, 401d to the DHP 406.

The End-User or Patient facing processing module may comprise any combination of a processor, memory, and/or software configured to perform the functions described herein for the End-User or Patient facing processing module. For example, the processor may be a general purpose processor programmed with computer executable instructions for implementing the functions of the End-User or Patient facing processing module. The processor may be implemented using a microprocessor or microcontroller configured to perform the functions of the End-User or Patient facing processing module. The processor may be implemented using an embedded processor or digital signal processor configured to perform the functions of the End-User or Patient facing processing module.

As introduced above, the user devices 402a, 402b may each include an End-User or Patient facing processing module to respectively process and analyze the inhaler data received from the paired inhalers to determine and/or extract the usage parameters associated with the inhalers 401a-d (e.g., user device 402a may process and analyze data relating to inhalers 401a, 401b, and user device 402b may process and analyze usage data relating to inhalers 401c, 401d). The End-User or Patient facing processing module may further process the inhaler data to identify and/or classify a given usage events into a certain category of usage event based on the usage parameters that are associated with the usage event. For example, the categories of usage events may include: no inhalation events, low inhalations events, good inhalation events, excessive inhalation events, exhalation events, actuation events, error events, underuse events, overuse events, inhaler cap openings or medication priming (e.g., such as opening of a blister strip, the opening of a capsule, etc., as described further herein), etc. The End-User or Patient facing processing module may also identify the type of inhaler associated with a given usage event. For example, the End-User or Patient facing processing module may identify usage events received from rescue inhalers as rescue usage events, and identify usage events received from maintenance inhalers as maintenance usage events.

As described herein, the End-User or Patient Facing processing module may classify a given usage event based on the respective inhalation parameters associated with the inhalation event (e.g., peak inhalation airflow parameters). In addition the End-User or Patient facing processing module may enrich or relate the usage events with timestamping and geolocation information, which, as further described herein, may be used by the DHP 406 to provide additional analytics to the End-User or Patient. The user devices 402a, 402b may include a display device and software (e.g., End-User or Patient facing processing module) for visually presenting or displaying the inhaler data associated with a given usage parameters and/or data related to usage events through a graphical user interface (GUI) on the display device. The user devices 402a, 402b may also provide alerts or notification via the GUI and/or via the inhalers 401a-d by sending an inhaler a message that causes the inhaler to illuminate a light source of the inhaler and/or generate an audible alert by way of a speaker of the inhaler.

The use determination systems of each of the inhalers 401a-d may detect usage events and determine usage parameters (e.g., airflow rate values, relative timestamps, etc.) associated with the detected usage event. Then the transmission modules of the inhalers 401a-d may transmit the detection of the usage event, along with the associated usage parameters (e.g., together referred to as inhaler data) to the End-User or Patient facing processing module running at a respectively paired user device. Referring to FIG. 4A, for example, inhaler 401a may detect a usage event, such as an inhalation, and determine usage parameters associated with the usage event (e.g., relative timestamp of the usage event, inhalation parameters, such as inhalation flow rate values, etc.). Then using its transmission module, the inhaler 401a may transmit the usage event and the associated inhaler data to the End-User or Patient facing processing module of user device 402a (e.g., the user device paired with inhaler 401a). The same or substantially similar process may occur when any one of the inhalers 401b-d detect a usage event.

After receiving the inhaler data from a respective inhaler, the End-User or Patient facing processing module may perform certain calculations and/or conversions on the received inhaler data. For example, the End-User or Patient facing processing module may convert a relative timestamp of the detected usage event to a local time stamp (e.g., based on the local time at which the usage event occurred). Similarly, the End-User or Patient facing processing module may convert the received airflow rate values (e.g., flow profile) to one or more inhalation parameters. For example, the End-User or Patient facing processing module may convert the received airflow rate values to one or more of a peak inhalation flow (PIF) value, an inhalation volume, an inhalation duration time, and/or a time to PIF value. The airflow rate values and/or respective conversions may be used to assess the user's compliance, which may provide valuable insight into the state of the user's respiratory disease (e.g., the likelihood that the patient is to experience an exacerbation event) and/or the user's sensitivity to current or changing environmental conditions (e.g., heat, air quality, humidity, etc.).

In addition, the End-User or Patient facing processing module may enrich or relate the inhaler data with a geotag, which may indicate of the location where the usage event occurred, such as the location of the user device when the usage event occur or the location of the user device when the usage event was received by the user device. Geotagging of the usage event may also, or alternatively, occur at the use determination system of the respective inhaler (e.g., doing so, however, may be computationally and/or power consumption wise, expensive as the inhaler may include a geographic positioning chipset or complex processing to triangulate its location).

In addition to geotagging enrichment, the End-User or Patient facing processing module may enrich or relate the inhaler data with other data associated with the user (e.g., other data that is relevant to the user's disease). For example, the End-User or Patient facing processing module may enrich or relate inhaler data with health or fitness data associated with the user. The health or fitness data associated with the user may, for example, be retrieved from one or more third party applications running on the user device (e.g., Apple Health app) or another user device (e.g., FitBit, Apple Watch, etc.). For example, the End-User or Patient facing processing module may enrich or relate the inhaler data with one or more of: a body mass index (BMI), heartbeat rate, weight, height, steps taken (e.g., daily average of steps), calories burned, hours of sleep, etc. Also, or alternatively, the enrichment of inhaler data with other health data may be performed by the DHP 406.

The End-User or Patient facing processing module may further classify a given usage event into usage event category, for example, based on the usage parameters associated with a given usage event. For example, as described herein, the categories of inhalation events may include: no inhalation events, low inhalations events, good inhalation events, excessive inhalation events, exhalation events, actuation events, error events, underuse events, overuse events, inhaler cap openings or medication priming events (e.g., such as opening of a blister strip, the opening of a capsule, etc., as described further herein), etc.

In certain instances, as described herein, the End-User or Patient facing processing module may forward the inhaler data it receives from a paired inhaler and any calculations or classification it performs based on the inhaler data to the DHP 406. For example, the End-User or Patient facing processing module may forward the inhaler data to the DHP 406 via one or more application programming interfaces published by the DHP 406. Before forwarding any information to the DHP 406, however, the End-User or Patient facing processing module may request consent or acknowledgment from the user to forward their data to the DHP 406.

Referring again to FIG. 4A, the End-User or Patient facing processing module may prompt a user for feedback. For example, the End-User or Patient facing processing module may prompt to user to provide self-assessment, relating to how the patient or subject is feeling (e.g., particularly in relation to the symptoms of the subject's respiratory disease). The End-User of Patient facing processing module may request this self-assessment periodically (e.g., daily, weekly, monthly, etc.). In a non-limiting example, a user may provide their self-assessment by selecting a self-assessment value from a range of self-assessment values. For example, the range of self-assessment values may be indicated by certain icons (e.g., such as emoji-type icons), and the user may select one of icons to indicate how the patient or subject is feeling on a given day. And, like it does for inhaler data, the End-User or Patient facing processing module may enrich or relate the self-assessment values with a geotag to indicate the location at which the patient or subject provided the self-assessment value. The End-User or Patient facing processing module may also associate a time with the self-assessment response. Further, in some examples, the End-User or Patient facing processing module may associated the self-assessment with one or more usage events based on time associated with the self-assessment response. Again, the End-User or Patient facing processing module may store this self-assessment information, as well as forward it to the DHP 406 (e.g., if the user provided consent or authorization) for aggregation and/or analysis. The End-User or Patient facing processing module may also generate one or more reports for the subject or user based on the received self-assessment values.

The End-User or Patient facing processing module may also distinguish the encrypted data received from a first inhaler with the encrypted data received from a second inhaler. Referring again to FIG. 4A, for example, the user device 402a may distinguish between the encrypted data received from inhalers 401a, 401b, and the user device 402b may distinguish between the encrypted data received from inhalers 401c, 401d. For example, the End-User or Patient facing processing module running on a user device 402a determines first usage information relating to the medicament delivered by the inhaler 401a based on the encrypted data received from the inhaler 401a, and determines second usage information relating to the medicament delivered by the inhaler 401b based on the encrypted data received from the inhaler 401b. Similarly, the End-User or Patient facing processing module running on a user device 402b determines third usage information relating to the medicament delivered by the inhaler 401c based on the encrypted data received from the inhaler 401c, and determines fourth usage information relating to the medicament delivered by the inhaler 401d based on the encrypted data received from the inhaler 401d. The End-User or Patient facing processing module running on the user devices 402a, 402b may then control a respective user interface to communicate or display the first, second, third, and fourth usage information to the End-User or Patient. In addition, the End-User or Patient facing processing module running on the user devices 402a, 402b may respectively forward the encrypted data received from the inhalers 401a-d (e.g., or a subset thereof) to the DHP 406. And, as further described herein, the DHP 406 may aggregate and perform analysis on the encrypted data forwarded from inhalers 401a-d/End-User or Patient facing processing modules running on user devices 402a, 402b.

The End-User or Patient facing processing module of the user devices 402a, 402b may include a general purpose processor, a special purpose processor, a DSP, a microcontroller, an integrated circuit, and/or the like that may be configured using hardware and/or software to perform the functions described herein for the End-User or Patient facing processing module. The End-User or Patient facing processing module may include a power supply and/or a battery. In some examples, the End-User or Patient facing processing module may reside partially or entirely on any combination of the user devices 402a, 402b (e.g., a smartphone, tablet, or personal computers) and/or remote servers.

Encryption of the data transmitted between the inhalers 401a-d and the use devices 402a, 402b in this manner enables transmission of the respective usage parameters. The encryption may, for instance, further enable such transmission to be effected securely, since decryption of the respective data is implemented by the End-User or Patient facing processing module configured to receive the encrypted data from the respective transmission modules of the inhalers 401a-d. The End-User or Patient facing processing module may, for example, be paired to the respective transmission modules of the inhalers 401a-d, such that the End-User or Patient facing processing module is configured, e.g. exclusively configured, to decrypt the encrypted data received from a paired inhaler. Thus, such encryption may enable secure transmission of the respective usage parameters to the End-User or Patient facing processing module running on a paired user device, which secure transmission may be preferred in the context of transmission of medical data relating to inhaler usage.

Whilst the respective transmission modules of the inhalers 401a-d are configured to transmit the encrypted data, in some non-limiting examples the respective transmission modules may be further configured to receive data, for example from the End-User or Patient facing processing module to which the encrypted data is sent. In such examples, the respective transmission modules of the inhalers 401a-d may be regarded as a transceiver, in other words as a transmitting and receiving module.

A clock module may, for example, be included in each of the respective inhalers 401a-d for assigning a time, for example a time stamp, to the usage parameter of the respective inhaler. The clock module may be implemented via a processor or other type of integrated circuit. The End-User or Patient facing processing module running on the user devices 402a, 402b may be configured to synchronize the clock modules of the respectively paired inhalers 401a-d. The respective clock modules may, for instance, receive time data transmitted from the End-User or Patient facing processing modules of the paired user device, e.g. via the respective transmission module of the user device. Such synchronization may, for instance, may provide a point of reference which enables the relative timing of use of the respective inhalers to be determined, which may have clinical relevance. For example, failure to inhale a maintenance medicament during a particular time period in which such an inhalation was or such inhalations were scheduled according to a treatment regimen may be correlated with increased rescue medicament usage towards the end of, or subsequently to, that time period of non-adherence to the treatment regimen. Such diagnostic analysis, which may be performed at the End-User or Patient facing processing module running on the user devices 402a, 402b or at the DHP 406, may be possible when the clock modules of the respective inhalers are synchronized with its paired user device.

Further, it should be appreciated that in some examples, the clock module of an inhaler may operate as an internal counter. When operating as an internal counter, the clock module may provide a relative count (e.g., as opposed to providing a mean solar time, such as a local mean time). For instance, the use determination system of an inhaler may start an internal counter (e.g., which counts up from 0 indefinitely) when, for example, the use determination system is woken out of an energy-saving sleep mode for the first time (e.g., after the mouthpiece cover is opened for the first time). Thereafter, any time-and-date stamp generated by the use determination system may be a relative time (or count) based on the internal counter of the clock module. The use determination system may periodically update the system clock every 250 microseconds (μs).

The End-User or Patient facing processing module may, in some examples, comprise a further clock module. The clock modules of each of the respective inhalers may thus be synchronized according to the time provided by the further clock module. The further clock module may, for instance, receive the time of the time zone in which the End-User or Patient facing processing module is situated. The End-User or Patient facing processing module may, for example, transmit the time of the time zone to the respective clock modules of the inhalers 401a-d, thereby to permit the clock modules to be synchronized according to the time in which the subject and their respective inhalers are located. Time stamping of the respective usage information may thus correspond to the time of day or night at the subject's geographical location. In addition, the respective usage information may be geotagged, to indicate a location of where the usage event occurred. This is particularly advantageous given the relevance of, for example, night time rescue medicament use to the risk of an impending respiratory disease exacerbation. The system may thus, for example, monitor the day time, night time, and/or location of rescue inhaler usage of a subject who has travelled across time zones. Alternatively or additionally, reminders issued by the system 400 (e.g., based on analytics performed at the DHP 406) to remind the subject to administer a maintenance medicament may account for the time of day or night at the subject's location.

The End-User or Patient facing processing module is configured to distinguish between the first encrypted data and the second encrypted data. First usage information relating to the first medicament is determined by the End-User or Patient facing processing module based on the distinguished first encrypted data. Similarly, second usage information relating to the second medicament is determined by the End-User or Patient facing processing module based on the distinguished second encrypted data. Referring again to FIG. 4A, user device 402a may be configured to distinguish between the encrypted data received from inhaler 401a and the encrypted data received from inhaler 401b. Similarly, user device 402b may be configured to distinguish between the encrypted data received from inhaler 401c and the encrypted data received from inhaler 401d

In an example, the End-User or Patient facing processing module receives a first identifier assigned to the first medicament (e.g., user device 402a receives a first identifier from inhaler 401a assigned to the medicament delivered by inhaler 401a). In such an example, a second identifier is assigned to the medicament delivered by another inhaler, such as inhaler 401b. In such an example, the first identifier is not associated with or assigned to the second medicament and the second identifier is not associated with or assigned to the first medicament. The End-User or Patient facing processing module receives the respective identifiers form the paired inhalers, and uses the respective identifiers to distinguish between the first encrypted data and the second encrypted data.

The respective identifier may, in certain examples, also denote further information, such as the dose strength of each dose delivered by the respective inhaler, for example via a suitable dose metering assembly included in the inhaler. Alternatively or additionally, the respective identifier may denote the total number of doses of the respective medicament contained by the respective inhaler (prior to first use), in other words in the medicament reservoir of the respective inhaler as supplied.

The End-User or Patient facing processing module may, for instance, be accordingly configured to recognize the dose strength and/or the total number of doses which can be provided by the respective inhaler from the respective identifier. Moreover, when the respective identifier denotes the dose strength, the End-User or Patient facing processing module may be configured to, for example, control the user interface to issue a notification that the label recommended dosages have been exceeded based on the respective usage information, in this case uses of the respective inhaler, and the respective dose strength.

In some examples in which a further inhaler configured for dispensing a medicament is added to the system which already includes an existing inhaler which dispenses the same medicament, the End-User or Patient facing processing module may be configured to determine, based on the respective identifiers for the existing inhaler and the further inhaler whether the dose strength of the further inhaler is the same as or different from that of the existing inhaler. If the respective dose strengths are different from each other, the End-User or Patient facing processing module controls a user interface to issue at least one notification. The at least one notification may, for example, comprise a notification informing the subject that the dose strength of the further inhaler is different from that of the existing inhaler and/or a notification to request that the subject discards the existing inhaler. In this manner, the system may assist the subject to adjust to a prescription change. The medicament in this example may be a rescue medicament or a maintenance medicament.

When the respective identifier denotes the total number of doses contained by the respective inhaler, the End-User or Patient facing processing module may be configured to control the user interface to issue a notification that the respective inhaler should be replaced based on the respective usage information, in this case uses of the respective inhaler, and the respective total number of doses as denoted by the respective identifier. For instance, subtraction of the number of uses of the respective inhaler as determined via the use determination system from the total number of doses denoted by the respective identifier will provide the number of doses remaining in the respective inhaler. The notification may be triggered by the End-User or Patient facing processing module when the determined number of doses remaining in the respective inhaler reaches or becomes lower than a predetermined threshold number of doses.

In a non-limiting example, the first identifier is included in a first key which is used to pair the first inhaler and the End-User or Patient facing processing module. The End-User or Patient facing processing module is configured to identify the first inhaler as an inhaler which delivers the first medicament on the basis of the first identifier included in the first key. Similarly, the second identifier may be included in a second key for pairing the second inhaler and the End-User or Patient facing processing module, and the End-User or Patient facing processing module identifies the second inhaler as an inhaler which delivers the second medicament on the basis of the second identifier included in the second key. In this manner, the first encrypted data is linked to the first medicament, and the second encrypted data is linked to the second medicament.

More generally, by the End-User or Patient facing processing module distinguishing between the first encrypted data and the second encrypted data, the first encrypted data relating to administering of the first medicament is processed separately from the second encrypted data relating to administering of the second medicament. Because the first and second medicaments are different from each other, and thus may each be associated, for instance, with distinct treatment regimens and/or administration protocols, this separate data processing may advantageously ensure that the first usage information relating to the first medicament does not become conflated with the second usage information relating to the second medicament. The system nevertheless permits consolidated handling and communicating of the first and second usage information.

In some examples, the End-User or Patient facing processing module is configured to control the user interface to communicate, for example display, the first and second usage information. In this way, the subject is informed of their usage of the first and second medicaments respectively. In the case of the first or second medicament being, for instance, a rescue medicament, the system may enable the subject to track the status of their respiratory disease. In the case of the first or second medicament being, for example, a maintenance medicament, the system may enable the subject to track their adherence to, or compliance with, a predetermined treatment regimen. In some examples, the End-User or Patient facing processing module is configured to control the user interface to display the first and second usage information simultaneously, such as in a single graphical user interface (GUI).

Further, in some examples, the End-User or Patient facing processing module may include a computer program that resides, for example, on the user device. The computer program comprises computer program code which is adapted, when the computer program is run on a computer, such as the user device, to implement the methods and technique described herein. The embodiments described herein for the system are applicable to the method and the computer program. Moreover, the embodiments described for the method and computer program are applicable to the system.

As described herein, the End-User or Patient facing processing module running on a user device may be configured to distinguish between the encrypted data received from paired inhalers. Distinguishing between the data received from the paired inhalers may allow the End-User of Patient facing processing module to separately display each of the usage event based on the inhaler associated with the usage event. For example, as described herein, the End-User or Patient facing processing module may distinguish between the usage events based on a respectively assigned identifier for each of the paired inhalers.

In a non-limiting example, the End-User or Patient facing processing module determines a use and/or system error based on the encrypted data received from one or more, e.g. each, of the paired inhalers. A use and/or system error may be a type of usage event. Such a use error may, for example, be indicative of potential misuse of the respective inhaler or inhalers. The system error may be indicative of a fault with a component of the respective inhaler, such as the use determination system and/or the transmission module of the respective inhaler. A system error may, for example, include a hardware fault of the respective inhaler. The user interface may be controlled by the processing module to provide an alert and/or notification based on the determined use and/or system error (e.g., such as providing an alert and/or notification for the determined use and/or system errors of each of a plurality of different inhalers, potentially including different medicaments). A use error may, for example, include a low inhalation event, a no inhalation event, and/or an excessive inhalation event.

A use error may alternatively or additionally include one or more of: the mouthpiece cover being left open for more than a predetermined time period, e.g. 60 seconds; multiple inhalations being recorded in respect of a single actuation of the above-described mechanical switch, for example a second inhalation performed within the same mouthpiece cover open/closed session; and an exhalation through the flow pathway, as determined from a positive pressure change being sensed in the flow pathway.

When the use error relates to the mouthpiece cover being left open for more than the predetermined time period, the inhaler's detection circuitry may only stay active for the predetermined time period to preserve battery life. This may mean that anything which would otherwise be detectable/determinable by the use determination system that occurs outside of this predetermined time period is not detected/recorded. Notifying the user of this error may therefore serve the purpose of informing the user that otherwise detectable events are not detected outside the predetermined time period triggered by opening of the mouthpiece cover.

It is noted that the abovementioned exhalation-based use error may not be recorded if such an exhalation is sensed subsequently to an inhalation being performed in respect of a given actuation of the mechanical switch, e.g. within the same mouthpiece cover open/closed session.

System errors may include one or more of: a problem occurring when saving inhalation data to a memory included in the inhaler, such as a memory included in the use determination system (“corrupted data error”); an error with the clock module of the inhaler (“time stamp error”); and an error relating to collecting information about the inhalation (“inhalation parameter error”).

In a particular example, use and/or system errors from more than one, e.g. all of, the inhalers included in the system are collected, e.g., aggregated, by a central processing module, such as the DHP 406. As further described herein, DHP 406 is further configured to provide alerts and/or notifications based on the collected use and/or system errors. For instance, the processing module controls the user interface to provide the alert and/or notification based on the number of use and/or system errors collected from the inhalers included in the system reaching or exceeding a predetermined number of use and/or system errors.

Although FIG. 4A illustrates the system 400 to include inhalers 401a-d, user devices 402a, 402b, DHP 406, and computer or sever 408, the system 400 may include additional or fewer instances of these devices. For example, the system 400 may include a plurality of additional inhalers like the inhalers 4001a-d. Further, each of these inhalers may be associated with a subject and/or user profile, and paired with a respective user device that, like the user devices 402a, 402b, include an End-User or Patient facing processing module, which may be configured to receive, and subsequently forward, inhaler data from the respectively paired inhalers. Similarly, the system 400 may include numerous additional computer or servers 408, which may be used to run a Care Provider facing processing module. That is, the example illustrated in FIG. 4A is a non-limiting example, and is simply intended to illustrate the types of devices that may be included in the system 400, and their respective roles/functions within the system 400.

FIG. 4B illustrates an example system 400a, which is another representation of the system 400 illustrated in FIG. 4A. As illustrated in FIG. 4B, the system 400a includes the DHP 406 and the computer or server 408 that comprises (e.g., runs or otherwise interacts with) a Care Provider facing processing module (e.g., also referred to herein as a Dashboard application) associated with a health care provider. The system 400a also include user devices 402c, 402d, each of which may include End-User or Patient facing processing modules. And, although not shown, the user devices 402c, 402d may each be paired with and receive inhaler data form one or more inhalers. Further, as described herein, the user devices 402c, 402d may forward this inhaler data, via the public or private network 404, to the DHP 406. As described herein, the DHP 406 may aggregate and perform analytics on the received inhaler data. For example, the DHP 406 may enrich and/or relate the inhaler data with information from one or more external sources 410, such as, weather data from and external weather API.

FIG. 4B is also illustrated to provide more detail on the DHP 406. For example, as illustrated in FIG. 4B, the DHP may include subsystems 406a, 406b. Each of the subsystems 406a, 406b may perform separate functions for the DHP 406. For example, each of the subsystems 406a, 406b may comprise databases or analytics engines that facilitate or perform the processing performed by the DHP 406, as described herein. As described herein, the subsystem 406a may be an operational subsystem, and the subsystem 406b may be an analytical subsystem. The DHP 406 may include additional components or subsystems. FIG. 4B is intended to illustrate some of the functions that the DHP 406 is configured to perform and how the DHP 406 performs said functions. That is, although the DHP 406 may be described using certain databases or subsystems, various other storage and processing techniques may also be used to configure the DHP 406.

As noted above, the DHP 406 is configured to receive and aggregate data from inhalers 401a-d (e.g., via a respectively paired user device), which may be associated with a plurality of different users. In some examples, the DHP 406 may reside on one or more servers, and may include computer software configured to perform the functions described in relation to the DHP 406. For example, the DHP 406 may provide data to Health Care Provider (HCP) facing processing module(s) (e.g., also referred to herein as a dashboard application) that may be accessible by a computer or server 408 associated with a health care provider, such as a hospital or hospital system, a health system, a medical group, a physician, a clinic, and/or a pharmaceutical company. In some examples, the dashboard application is a web application (e.g., a web portal) that may interact or otherwise communicate with the DHP 406, for example, via one or more communication interfaces published by the DHP. For example, the DHP is also configured to provide data, such as inhaler data, to clinicians and physicians through the use of the dashboard application (e.g., via the communication interface).

The DHP 406 is configured to receive and store inhaler data from End-User or Patient facing processing modules residing on user devices (e.g., the patient-facing mobile applications), and is configured to provide data to a Care Provider facing processing module residing on a computer or server associated with a health care provider (e.g., via the dashboard application). The inhaler data may include any of the data described with reference to the inhalers described herein, such as usage events, error events, inhalation profiles, associated timestamps, medicament types, etc. The inhaler data may be associated with an inhaler and/or a user profile, for example, at the processing module (e.g., residing on the user device) and/or at the DHP. As noted in detail below, the DHP may anonymize (e.g., de-identify, unassociated, disassociate) the inhaler data from a particular user, and the DHP may perform analytics on anonymized data relating to the inhalers, such that the DHP (e.g., or a user of the DHP) is unaware of the particular user that the inhaler data is associated with.

In many examples, the DHP 406 does not have a direct connection with the inhalers 401a-d, and as such, the DHP 406 is unable to verify (directly verify with the inhaler) the completeness of the usage data it has received. Further, in some examples, the DHP 406 cannot communicate to the inhaler, and the inhaler cannot communicate directly to the DHP 406. This creates a technical program as to how the DHP 406 can perform data verification without a line of communication with the inhaler that is capturing usage events. That is, as the DHP 406 is not in direct communication with the inhaler that is capturing the usage events, the DHP 406 is unable to perform data verification of the usage events.

The DHP 406 may be configured to enable, and in some instances initiate, the transfer of data (e.g., inhaler data) to or from the End-User or Patient facing processing modules (e.g., the processing modules residing on user devices). For example, the DHP 406 may expose a communication interface, such as a representational state transfer (REST) application programming interface (API), which may be used to access or update the data maintained by the DHP 406. Using the exposed communication interface, the DHP 406 may also be configured to enable, and in some instances initiate, the transfer of data (e.g., inhaler data and/or anonymized inhaler data) to or from the Care Provider facing processing modules (e.g., the processing modules running on server or computer 408). In response, the processing modules residing on the user devices may access the data through a communication interface published by the DHP. In some examples, the processing module on the user device may be configured to periodically send, such as every 15 minutes, inhalation data (e.g., or an indication that there is no new inhalation data) to the DHP 406, for example, via the communication interface published by the DHP 406. The DHP 406 may receive the patient's consent to access the patient's data via their respective processing module residing on their user device. In addition, the DHP 406 may also store incoming data from dedicated mobile and web applications, such as the End-User or Patient facing processing modules of the user devices 402a, 402b. Again, the DHP 406 may receive the patient's consent to access this information, incoming data, etc., through the published communication interface.

The DHP may publish one or more communication interfaces to allow the user devices to transmit inhaler data to the DHP 406 and to allow external devices, such as the user devices 402a-d and/or computer or server 408, to receive data from the DHP 406. The DHP 406 may publish differing communication interfaces based on the respective processing module that is attempting to communicate with the DHP 406. For example, the DHP 406 may publish a first communication interface designed to be used for the End-User or Patient facing processing module(s), and a second communication interface designed to be used by the Care Provider facing processing module(s) (e.g., also referred to herein as the Dashboard application).

The DHP 406 may publish a communication interface for the End-User or Patient facing processing modules (e.g., an End-User or Patient facing communication interface). And since the End-User or Patient facing processing modules are configured to transmit or forward the inhaler data it receives from respectively paired inhalers, the End-User or Patient facing communication interface may be designed such that it can be effectively used to communicate inhaler data as well as any calculation or analysis performed by the End-User or Patient facing processing module on the inhaler data. In some examples, the End-User or Patient facing communication interface may be designed to send and receive information in a predefined format, such as JSON.

The DHP 406 may publish the End-User or Patient facing communication interface to enable role based access checks (RBACs). For example, the End-User or Patient facing communication interface of the DHP 406 may enable RBACs to authorize incoming requests, such as to determine whether the user or End-User or Patient facing processing module making the request has access to perform the requested action. To enable RBACs, the End-User or Patient facing communication interface published by the DHP 406 may include an entity check, role check, a data access check, and/or a consent check. To perform each of these RBAC checks, the End-User or Patient facing processing module of a respective user device may transmit an indication of the user to the DHP 406 via the End-User or Patient facing communication interface.

The DHP 406 uses the entity check to determine whether a user-profile for the respective user of the End-User or Patient facing processing module (e.g., a user, or a guardian of the user) exists within the DHP 406 (e.g., within the operational subsystem 406a). In order to perform the entity check, the End-User or Patient facing processing module of a user device may transmit an indication of the user (e.g., the user of the End-User or Patient facing processing module). The DHP 406 may receive the indication of the user and determine is a user profile exists for that user within the operational subsystem 406a. If a user profile does not exist, the DHP 406 may reject the request. However, the End-User or Patient facing communication interface published by the DHP 406 may also include an exception to the entity check, such as the ability to add a user profile to the operational subsystem 406a. As a result, if a user profile does not exist for the user, the End-User or Patient facing processing module may transmit a request to the DHP 406 to add a user profile for the user using the End-User or Patient facing communication interface.

The DHP 406 may use the role check to determine if a user of the End-User or Patient facing processing module has the correct privileges to access the DHP 406. For example, in order to access the DHP 406, the user of the End-User or Patient facing processing module must be either a user within the operational subsystem 406a or a parent or guardian of a user.

The End-User or Patient facing communication interface may include a data access check, which may be used to determine if the user of the End-User or Patient facing processing module has access to the data they are requesting/modifying. For example, the data access check may be used to ensure that a patient or respective guardian is only able to access/modify their own data. That is, if the user is a guardian or a patient, the data access check may also verity that the guardian is only able to access/modify either their own or their dependents data.

The End-User or Patient facing communication interface published by the DHP 406 may include a consent check, which may be used to determine if the user has consented or authorized their data to be stored or maintained within the DHP 406. If the user has not provided consent, the consent check may be rejected by the DHP 406. There may, however, be an exception to the consent check, such as the ability to modify consent. The End-User or Patient facing processing module may, in response to a user selection of such, transmit an indication to the DHP 406 via the published End-User or Patient facing communication interface to modify the consent provided by the user (e.g., consent or authorize that their information may be stored or maintained within the DHP 406).

After a user has cleared all of the relevant RBACs, the DHP 406 may transmit, via the End-User or Patient facing communication interface, an authentication token to the End-User or Patient facing processing module. The token may be valid for a certain period of time, after which the End-User or Patient facing processing moldy may be required to re-clear the relevant RBACs provided by the DHP 406. While the token is valid, however, the End-User or Patient facing processing module may include the received token with all of the future request made to the DHP 406.

The End-User or Patient facing communication interface published by the DHP 406 may enable the End-User or Patient facing processing module to add a usage event (e.g., an inhalation event) to the DHP 406. In order to add a usage event to the DHP 406, a user profile for the user of the End-User or Patient facing processing module must be stored within the operational subsystem 406a (e.g., or a user profile for a parent or guardian of the user).

In order to add a usage event to the DHP 406, an End-User or Patient facing processing module may transmit an add usage event request to the DHP 406 that includes a valid token. In addition, the add usage event request may include an identifier of the relevant medical device (e.g., a serial number of the relevant inhaler); an identifier of the prescription for the relevant medical device (e.g., which may indicate the users level of adherence or compliance); a time associated with the usage even (e.g., the time at which the inhalation event occurred); and/or the various usage parameters/analysis or calculations performed based on the usage parameters described herein. In response to receiving the add usage event request via the End-User or Patient facing communication interface, the DHP 406 may retrieve the user profile for the user from the operational subsystem 406a, and confirm that the identifier of the relevant medical device and the identifier of the prescription for the relevant medical device match a medical device/prescription in the user profile. In addition, the DHP 406 may determine whether the time associated with the usage event render the usage event stale. For example, a usage event may be considered stale if the time associated with the usage event is greater than a predefined period of time (e.g., 59 seconds) from the current time.

The End-User or Patient facing communication interface published by the DHP 406 may enable the End-User or Patient facing processing module to retrieve data from the DHP 406. As described herein, in order to retrieve data from the DHP 406, the user of the End-User or Patient facing processing module must be user within the operational subsystem 406a or a parent or guardian of a user.

In order to perform data retrieval, an End-User or Patient facing processing module may transmit a data retrieval request to the DHP 406 that includes a valid token. In response to the data retrieval request and valid token, the DHP 406 may determine the last time that the user retrieved data from the DHP 406 (e.g., based on an inhalerSynchTime field). The DHP 406 may then, via the End User or Patient facing communication interface, return all the relevant data it has received since the last time the user retrieved data from the DHP 406. For example, the DHP 406 may return one or more of the following: user profile information; medical Device information; mobile device information; Prescription Information; Medication Orders; User Preferences Settings; Medication Administration (e.g., usage events, inhalation events, etc.); Questionnaire Response (e.g., self-assessment responses), and/or the like.

The End-User or Patient facing communication interface published by the DHP 406 may enable the End-User or Patient facing processing module to emancipate a user that is a minor. In order to emancipate a user that is a minor, the user of the End-User or Patient facing processing module must be given the authority to emancipate an individual (e.g., an administrator of the DHP 406a).

In order to perform an emancipation, an End-User or Patient facing processing module may transmit an emancipation request to the DHP 406 (e.g., via the published End-User or Patient facing communication interface) that includes a valid token (e.g., a valid token that indicates that the user is an administrator of the DHP 406). In addition, the emancipation request may include an identifier of a relevant medical device (e.g., serial number of an inhaler). For example, the identifier of the relevant medical device may be used look up or retrieve the user profile of the user that is a minor (e.g., from the operational subsystem 406a).

The emancipation request may also include an address (e.g., an email address) to which to associate the minor's user profile with. The emancipation request may further include the date of birth of user to be emancipated. The DHP 406 may then verify that the date of birth included in the request matches the date of birth in the retrieved user profile. In addition, the DHP 406 may verify that the user is allowed to be emancipated. The age at which emancipation is allowed may vary based on the jurisdiction or location of the user. That is, in some locations a user must be 18 years of age to be emancipated, while in other locations the age for emancipation may be 16 years old, 19 years old (e.g., Alabama and Nebraska), or 21 years old (e.g., Mississippi). Accordingly, the DHP 406 may determine the user jurisdiction or location and confirm that their date of birth complies with the rules of emancipation for that jurisdiction or location. If emancipation is allowed, the DHP 406 may accept the emancipation request and update the user profile for that user to indicate that they are no longer to be considered a minor.

Although described as receiving the inhaler data from the End-User or Patient facing processing modules running on the user devices 402a, 402b, in some examples, the DHP 406 may receive the data directly from the inhalers 401a-d themselves, such as in instances where the transmission modules on the inhalers 401a-d include cellular chipsets that are capable of communicating directly with the DHP 406.

Referring to FIG. 4B, the subsystem 406a may be an operational subsystem. The operational subsystem 406a is responsible for supporting the End-User or Patient facing processing modules running on user devices (e.g., user device 402a-d). The operational subsystem 406a may also support the HCP facing processing modules of a computer or server associated with a care provider (e.g., the computer or server 408). As described herein, the End-User or Patient facing processing modules and the HCP facing processing modules may be implemented in the form of a mobile application (e.g., installed at a user device or computer/server) or a web applications (e.g., run or executed by visiting a web page via a browser). For example, the End-User or Patient facing processing modules may designed for a user to send and receive data (e.g., inhaler data), and/or otherwise interact with the DHP 406, for example, via the communication interface published by the DHP 406. The HCP facing processing modules (e.g., also referred to as the Dashboard application) may designed as a web application that is configured to support care providers (e.g., health care practitioners and/or health care professionals), and may allow them to access patient data specific to a “program” to which a patient or subject has consented. Like the End-User or Patient facing processing modules, the HCP facing processing modules or Dashboard application may send and receive data or otherwise interact with the DHP 406 using the communication interface published by the DHP 406.

The DHP 406 is also configured to provide the analyzed and manipulated data (e.g., the weather enrichment, a compliance score, a future compliance score, a risk score, etc.) back to the user devices 402a, 402b, or to the computer 408 that includes the HCP facing processing module associated with a health care provider. Also, or alternatively, the DHP 406 and/or the user devices 402a, 402b may perform control of one or more smart devices installer at the End-User or Patient's residence. For example, the DHP 406 and/or the user devices 402a, 402b may perform control of a smart thermostat or HVAC system installed at the End-User or Patient's residence, for example, as described in more detail herein.

The information maintained by operational subsystem 406a may be organized by user profile. For example, as described herein, a user profile may be created for each user (e.g., subject or patient). Among other things, the user profile may identify the user of the inhaler(s) (e.g., indicate the name, age, etc. of the user). In addition, a user profile may include the inhalers that are associated with the user (e.g., prescribed to the user). For example, the user profile may indicate each of the inhalers prescribed to the user, the medicament delivered by each of the respective inhalers, the dosage regiment associated with each of the respective inhalers, the associated user devices (e.g., mobile applications), an individualized compliance score for the user, an individualized future compliance score for the user, an individualized risk score for the user, their personal information (e.g., date of birth, gender, etc.), their HCP information, etc. A respective user profile may also include the relevant data (e.g., usage data, self-assessment responses, etc.) for a respective user. For example, the user profile may include a list of all of the usage events (e.g., inhalations), as well as the respective inhaler data associated with a given usage event (e.g., inhalation parameter(s), classification of inhalation event, time, location, etc.). Further, in some examples, the user profile may be generated using enriched usage events (e.g., including enriched usage parameters).

A user profile may also include supplemental information about the user gathered by the DHP 406. For example, a user profile may include the user's daily self-assessment responses, which may be periodically (e.g., daily, weekly, monthly, etc.) provided by the user to the DHP 406 via the End-User or Patient facing processing module running at a user device.

A user profile for a given user may also be provided with access or consent to view the user profile for another user. For example, a user profile for the parent of a user who is a minor may be provided with the access to view the user profile of that user (e.g., the parent of a patient may be provided with the ability to access the user profile of their child).

The DHP 406 may characterize or perform further calculations on the inhaler data received from the End-User or Patient facing processing modules of the user devices 402a-d. The End-User or Patient facing processing modules, as described herein, may forward the inhaler data received from a respectively paired inhaler (e.g., and any additional calculations or analysis performed by the End-User or Patient facing processing modules) to the DHP 406. In response to receiving the inhaler data, the DHP 406 may identify the user associated with the inhaler data and store the inhaler data in the user profile of that user. For example, as described herein, the user profiles maintained by the DHP 406 may be stored in the operational subsystem 406a. Further, in some examples, the DHP 406 may calculate additional inhalation parameters from the received inhalation parameters (e.g., from an inhalation flow profile) for a usage event. In addition, the DHP 406 may perform analytics on the data (e.g., or a subset thereof) stored in the operational subsystem 406a. For example, the DHP 406 may perform analytics on the data stored in the operational subsystem 406a to identify trends, which may then be used to detect or predict the likelihood of an event (e.g., an exacerbation by a user). For example, the DHP 406 may use the subsystem 406b to perform analytics on the data stored in the operational subsystem 406a. The operational subsystem 406a may send inhalation parameters to the subsystem 406b. The subsystem 406b may analyze the inhalation parameters to determine compliance and/or risk information (e.g., an individualized compliance score, an individualized future compliance score, and/or an individualized risk score). The subsystem 406b may send the determined compliance and/or risk information to the operational subsystem 406a.

In response to receiving the inhaler data, the DHP 406 is configured to analyze and manipulate the data. For example, and as further described herein, the DHP 406 may enrich or relate the received inhaler data with information from one or more external sources, such as, weather data from an external weather source. The DHP 406 may also aggregate and analyze the data to determine one or more metrics associated with the End-User or Patient, such as an individualized compliance score, an individualized future compliance score, and/or an individualized risk score. For example, the DHP 406 may send an alert to and/or send a notification for display on the user device associated with a user when their compliance score is below a threshold, future compliance score is below a threshold, and/or risk score exceeds a threshold. The threshold value for the compliance score, future compliance score, and/or risk score may be a predefined numerical value or percentage that is indicative of an increased risk of an exacerbation event (e.g., an asthma, COPD, or other respiratory exacerbation event). The alert may indicate that the user should maintain their rescue inhaler nearby. Additionally or alternatively, the alert/notification may indicate how the user can improve compliance with the prescribed treatment.

The DHP 406 may enrich inhaler data (e.g., inhalation parameters, such as inhalation flow information) received from a plurality of inhalers. Again, each of the plurality of inhalers may be associated with a respective user include a memory, a processor, a pressure sensor or acoustic sensor, and/or a wireless communication circuit. Further, a respective user may be associated with a plurality of inhalers (e.g., one or more rescue inhalers and/or one or more maintenance inhalers). The pressure or acoustic sensor of each inhaler may be configured to detect a use (e.g., an inhalation of the inhaler by the user) of the inhaler and sense or measure an inhalation parameter (e.g., a flow rate, a PIF, an inhalation volume, an inhalation duration, or a time-to-peak inhalation). The inhalation parameter may be used to assess the user's compliance, which may provide valuable insight into the state of the user's respiratory disease (e.g., the likelihood that the patient is to experience an exacerbation event) and/or the patient's sensitivity to current or changing environmental conditions (e.g., heat, air quality, humidity, etc.). Further, the processor may be configured to generate a usage event that comprises the inhalation parameter in response to each use of the inhaler by the user.

The DHP 406 may receive a plurality of usage events, for example, from each of the user devices 402a, 402b. Each usage event may include an associated time (e.g., the time at which the usage event occurred), one or more inhalation parameters for the usage event (e.g., PIF, an inhaled volume, an inhalation duration, or a time-to-peak inhalation), and/or a geographic location for the usage event. The DHP 406 may enrich or associate one or more environmental conditions to each inhalation parameter of each of the plurality of usage events using the respective time and geographic location associated with the usage events. For example, the DHP 406 may tag (e.g., geotag) each of the inhalation parameters of each of the plurality of usage events with the respective geographic location associated with the usage event. Tagging each inhalation parameter with their respective geographic location may allow for more accurate comparison and analysis of usage events. For example, geo-tagging inhalation parameters may allow for more accurate pattern detection, identification of environmental conditions that affect a particular user's inhalation parameters (e.g., and in turn their respiratory disease), such as humidity, temperature, pollen, pollution, etc. Further, the DHP 406 may enrich or relate self-assessment responses with environmental conditions. The environmental conditions may comprise any combination of temperature, humidity, outdoor air pollutant concentration, presence of particulate matter of 2.5 microns or smaller (PM2.5), presence of particulate matter of 10 microns or smaller (PM10), ozone concentration, nitrogen dioxide (NO₂) concentration, or sulfur dioxide (SO₂) concentration. In relating environmental conditions with various usage events, the DHP 406 may be able to identify relationships or correlations between the usage event and various environmental conditions, for example, to predict the user's sensitivity to current or changing environmental conditions, such as heat, air quality, humidity, etc. As such, the DHP 406 may be able determine an individualized risk score for a user, which may be used to predict the likelihood that the user experiences an exacerbation event in the future. The individualized risk score may be determined for the user using pattern detection, as described herein.

In some examples, the enrichment procedure may include querying one or more external weather sources to retrieve the environmental conditions present at the respective time and geographic location associated with the usage events or self-assessment responses. The DHP 406 may wait or delay initiating the enrichment procedures for usage events and self-assessment responses for a period of time. The time delay may differ based on the type of environmental condition being queried. For example, the time delay for querying certain environmental conditions (e.g., temperature and/or humidity) may be shorter than the time delay for querying other environmental conditions (e.g., pollen and/or air quality).

The subsystem 406b may be an analytical subsystem. In some examples, the analytical subsystem 406b may include anonymized data from the various user profiles maintained by the operational subsystem 406a. As described herein, the anonymized data within the analytical subsystem 406b may be used for data research purposes or other analytical queries. For example, the anonymized data within the analytical subsystem 406b may include the data (e.g., or a subset thereof) maintained within the operational subsystem 406a, except that any information that may be used to identify the user (e.g., name, age. etc.) may be removed or otherwise obfuscated (e.g., the identifiable information may be hashed). Further, the user may consent or otherwise provide approval of the respective information within their user profile is anonymized and subsequently stored within the analytical subsystem 406b. For example, the DHP 406 may also anonymize (e.g., disassociate) the inhaler data with a particular user profile prior to providing the data to the analytical subsystem. As further detailed herein, the DHP 406 may aggregate the anonymized inhaler data and perform analysis on the anonymized data, for example, to identify certain patterns (e.g., trends) in the aggregated anonymized data. The DHP 406 may then use the anonymized inhaler data and/or any identified patterns (e.g., trends) to perform analysis on the inhaler data associated with a given user, for example, to determine an individualized compliance score, an individualized future compliance score, and/or an individualized risk score for the user. However, that is not always the case, and in other examples, the DHP 406 may provide identified data to the analytics subsystem.

The DHP 406 may include additional components or subsystems, and FIG. 4B is not intended to be a limiting example. Rather, FIG. 4B is intended to illustrate some of the functions that the DHP 406 is configured to perform and how the DHP 406 performs said functions. That is, although the DHP 406 may be described using certain databases or subsystems, various other suitable techniques may also be used to configure the DHP 406.

Access to the analytical subsystem 406b may be subject to strict authentication protocols. For example, only certain analysts or clinicians may be granted access to the analytical subsystem 406b. Additionally, the operational subsystem 406a may be granted access to certain information maintained by the analytical subsystem 406b. After being granted access to the analytical subsystem 406b, an analyst or clinician may run various test or procedures based on the anonymized data within the analytical subsystem 406b. For example, an analyst or clinician may use the anonymized data within the analytical subsystem to develop new treatments, medicaments, analytical procedures, etc.

The analytical subsystem 406b may also employ machine learning and/or predictive modeling techniques. The analytical subsystem 406b may comprise one or more machine learning algorithms, such as but not limited to, an instance-based algorithm (e.g., k-Nearest Neighbor (kNN), Learning Vector Quantization (LVQ), Self-Organizing Map (SOM), Locally Weighted Learning (LWL), Support Vector Machines (SVM), etc.), a regression algorithm (e.g., a linear regression algorithm, a logistic regression algorithm, etc.), a decision tree algorithm, a Bayesian algorithm (e.g., a Naive Bayes classifier), an ensemble algorithm (e.g., a weighted average algorithm), etc.

The analytical subsystem 406b may train the algorithm using the historical anonymized data received from the inhalers. The analytical subsystem 406b may train the algorithm using training data by way of an unsupervised learning method or a supervised learning method, such as, but not limited to a gradient descent or a stochastic gradient descent learning method. An unsupervised learning method may be a type of machine learning that does not use labels for training data. The unsupervised learning method may be a learning method for learning artificial neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters.

A supervised learning method may use labeled training data to train the machine learning algorithm. As training data is received, the supervised learning method may adjust weights until the machine learning algorithm is appropriately weighted. The supervised learning method may measure the accuracy of the machine learning algorithm using a loss function. The supervised learning method may continue adjusting the weights until the error is reduced below a predetermined threshold. The supervised learning method may comprise gradient boosted decision trees. Gradient boosted decision trees may combine weak learners to minimize the loss function. For example, regression trees may be sued to output real values for splits and to be added together. The weak learners may be constrained, for example, to a maximum number of layers, a maximum number of nodes, a maximum number of splits, etc. Trees may be added one at a time to the machine learning algorithm and existing trees may remain unchanged. A gradient descent procedure may be used to minimize loss when adding trees. For example, additional trees may be added to reduce the loss (e.g., follow the gradient). In this case, the additional tree(s) may be parameterized and those parameters may be modified to reduce the loss. The supervised learning method may comprise an XGBoost algorithm. The XGBoost algorithm may comprise an implementation of gradient boosted decision trees that is designed for speed and/or performance. The XGBoost algorithm may automatically handle missing data values, support parallelization of tree construction, and/or continued training.

The training data may include any combination of labeled and unlabeled data. For example, the historical anonymized data received from the inhalers may be an example of labeled data. That is, the data may include any combination of data received from an inhaler or mobile device, or calculated by the operational subsystem 406a, such as a day, time, and/or place of an inhalation event, inhalation parameter(s), biometric parameter(s), environmental conditions, daily self-assessment (SA) responses, etc. (e.g., any of the factors described with reference to Table 1). Further, the operational subsystem 406a may identify inhalation parameters, geographic location, and/or environmental conditions of usage events, for example, based on the received data from the inhalers. Further, the operational subsystem 406a may determine that a user experienced an exacerbation event based on manual data entry by the user and/or receipt of data from the health care provider.

The historical anonymized data within the analytical subsystem 406b may be input into various machine learning systems and/or predictive models. In response, the machine learning systems and/or predictive models may, based on the inputted anonymized data, be used to assess a user's compliance with a prescribed treatment and/or predict the likelihood of future events, such as the likelihood of a respiratory exacerbations event (e.g., asthma, COPD, or CF exacerbation) or a likelihood of future compliance with the prescribed treatment, as described in more detail herein. For example, the machine learning systems and/or predictive models may be used to detect the attributes, circumstances, and/or conditions (e.g., inhalation parameters, weather conditions, number of recent exacerbations, number or recent rescue and/or maintenance medicament usage events, health related data received from third parties, etc.) that lead to an increased likelihood of exacerbations and/or a lack of compliance for a particular user.

The DHP 406 may determine the individualized compliance score, individualized future compliance score, and/or the individualized risk score for each user associated with at least one usage event. In some examples, the score may be determined by the operational subsystem 406a, or by the analytical subsystem 406b using anonymized data (e.g., a combination of identified data for a given user and anonymized data associated with other users). As described herein, the compliance score may indicate how compliant a user has been with respect to the user of one or more of their inhalers, the future compliance score may indicate a prediction of how compliant the user will be going forward with respect to the use of on roe more of their inhalers, and the risk score may indicate the state of the user's respiratory disease (e.g., the likelihood that the patient is to experience an exacerbation event). The individualized compliance score, the individualized future compliance score, and/or the individualized risk score may indicate the user's sensitivity to current or changing environmental conditions (e.g., heat, air quality, humidity, etc.). Since the DHP 406 has access to inhalation parameters of the user, the scores determined by the DHP 406 may provide valuable insight into the state of the user's respiratory disease (e.g., the user's compliance, future compliance, and/or the likelihood that the patient is to experience an exacerbation event) and/or the patient's sensitivity to current or changing environmental conditions (e.g., heat, air quality, humidity, etc.).

The individualized compliance score, individualized future compliance score, and/or the individualized risk score may be based on the inhaler data of the inhalers associated with a respective user profile (e.g., certain inhalation parameter, such as peak inhalation flow, inhalation volume, etc.), analytics performed by the DHP 406 (e.g., analytics performed on the aggregated anonymized inhaler data maintained by the analytical subsystem 406b and/or any identified trends in the aggregated data) and/or the End-User or Patient facing processing modules, and/or any enrichment information performed by the DHP 406 and/or the End-User or Patient facing processing modules. For example, DHP 406 may perform analytics the aggregated anonymized inhaler data maintained by the analytical subsystem 406b to identify certain trends in the aggregated data. The DHP 406 may then determine whether any of the identified trends correlate with the inhaler data for a given user (e.g., determine whether the inhaler data associated with the user is similar to the anonymized inhaler data of other users). The DHP 406 may further use any correlations between the inhaler data for the user and the anonymized inhaler data of other users to assess the state of the user's respiratory disease (e.g., the likelihood that the patient is to experience an exacerbation event) and/or the patient's sensitivity to current or changing environmental conditions (e.g., heat, air quality, humidity, etc.).

As described herein, the analytical subsystem 406b may perform analytics on the aggregated anonymized inhaler data, for example, to identify certain patterns/trends. The analytics and/or identified trends may be shared between the analytical subsystem 406b and the operational subsystem 406a.

The individualized risk score may be determined from a range of risk scores, and indicate the likelihood that a given user is experience a certain medical event, such as an exacerbation event. For example, the higher an individualized risk score is determined to be, the higher the likelihood that the patient will experience an exacerbation event. Similarly, the lower an individualized risk score is determined to be, the lower the likelihood that the patient will experience an exacerbation event. For example, the risk score may be on a scale of one to ten, or one to 100, with higher numbers indicating a higher risk of exacerbation.

The DHP 406 may consider a variety of factors in determining a user's individualized compliance score, individualized future compliance score, and/or individualized risk score. Further, the factors considered in determining a risk score for a given patient or subject may be stored or otherwise indicated within the user profile for the user maintained by the operational subsystem 406a. For example, the factors considered in determining the individualized compliance score, the individualized future compliance score, and/or the individualized risk score may include the user's inhalation parameters associated with their usage events for rescue and maintenance usage events; the location, time of day, and corresponding weather information for usage events (e.g., maintenance events and/or exacerbation events); the types of the most recent usage events (e.g., maintenance verses rescue events); the frequency of exacerbations experienced by the user within a predetermined period of time, such as anywhere from the last day to the last week or month; the patient's adherence or compliance with a program prescribed to the user (e.g., the number of rescue and/or maintenance usage events by the patient over a predetermined amount of time, such as the past week or month); the user's self-assessment responses; the environmental factors of the current location of the user; the current time of day at of the location of the user; and any correlation between any of the factors provided above.

The individualized compliance score, the individualized future compliance score, and/or the individualized risk score may be determined using pattern detection. The DHP 406 may identify one or more patterns (e.g., trends) associated with a user using a supervised or unsupervised machine learning algorithm. The pattern associated with the user may be determined using the one or more of the factors provided herein. The DHP 406 (e.g., the machine learning algorithm) may identify a plurality of patterns associated with a plurality of users. The plurality of patterns associated with the plurality of users may be determined using the factors provided herein. The DHP 406 may compare the one or more patterns associated with the user to the patterns associated with the plurality of users. The DHP 406 may identify one or more similarities between the pattern associated with the user and one or more of the plurality of patterns associated with the plurality of users. The DHP 406 may identify those patterns of usage events (e.g., comprising inhalation parameters) that are most similar to the user's pattern, and may determine how those users of the identified patterns progressed in the time period subsequent to the identified pattern (e.g., became more compliance, experience exacerbations, etc.). Based on how the other users of the identified pattern progressed (e.g., based on their subsequent usage events), the DHP 406 may determine the individualized compliance score, individualized future compliance score, and/or the individualized risk score for the user. Further, the machine learning algorithm may associate a ranking with each of the one or more factors. As such, the machine learning algorithm may identify those factors that are of greater or lesser influence to the pattern detection (e.g., and ultimately the score, whether it be a compliance score, a future compliance score, or a risk score).

As noted above, the individualized compliance score, the individualized future compliance score, and/or the individualized risk score may be based on one or more environmental conditions associated with a present location of the user (e.g., based on the location of the user device 402a, 402b). For example, the individualized compliance score, the individualized future compliance score, and/or the individualized risk score may be based on the current environmental conditions of the location of the user and respective relationships between an environmental condition and inhalation parameter of the plurality of usage events of the user. As another example, the individualized compliance score, the individualized future compliance score, and/or the individualized risk score may also, or alternatively, be based on a comparison of a number of most recent inhalation parameters associated with the user to a historical average of the inhalation parameter for the user.

A DHP 406 may consider health and fitness data associated with the user when determining the individualized compliance score, the individualized future compliance score, and/or the individualized risk score for the user. For example, as described herein, a user's inhaler data may be enriched (e.g., by the End-User or Patient facing processing module) with certain health and fitness data associated with the user. The user's health or fitness data may include: a body mass index (BMI), heartbeat rate, weight, height, steps taken (e.g., daily average of steps), calories burned, etc. And, in turn, the DHP 406 may consider the user's health and fitness data when determining the individualized compliance score, the individualized future compliance score, and/or the individualized risk score.

In addition, a user's individualized compliance score, individualized future compliance score, and/or individualized risk score may also consider certain global factors, which may be maintained by the analytical subsystem 406b (e.g., factors identified from the plurality of users that authorize the DHP 406 to perform analytics on a anonymized set of their respective user profile). For example, a user's individualized risk score may consider the frequency of exacerbations of other subjects or patients in the same or substantially same location with the same or substantially the same weather conditions. The user's factors may be weighted more heavily than the factors relating to the general population when determining the user's individualized compliance score, individualized future compliance score, and/or individualized risk score.

As described in more detail herein, the DHP 406 may use machine learning, such as a predictive model (e.g., a decision tree model, a gradient boost model, adaboost mode, a weighted model, etc.) to determine the individualized compliance score, the individualized future compliance score, and/or the individualized risk score, for example, based on one or more inhalation parameters and environmental conditions. The predictive model may be based on one or more relationships, such as relationships between the various environmental condition and inhalation parameters of the plurality of usage events. For example, the predictive model used to determine the individualized score for a respective user may be based on one or more relationships (e.g., relationships between the various environmental condition and inhalation parameters of the plurality of usage events, relationships between one or more environmental conditions associated with a present location of the user, and relationships between the environmental condition and inhalation parameter of the plurality of usage events from other users). The predictive model may determine weights for each variable and/or relationship.

The weights of the various relationships of the predictive model may differ between users, for example, when generating an individualized score for that user. For example, the predictive model may comprise a higher weight applied to the relationship between the environmental conditions and each inhalation parameter that is associated with the user than the weight applied to the relationship between the environmental conditions and each inhalation parameter that is associated with one of the other users.

Further, the predictive model used by the DHP 406 may weigh factors (e.g., such as inhalation parameters, weather or environmental conditions, and/or usage trends) differently in the prediction model based on the user's historical sensitivity to these factors, and the result of such individualized weighting may generate the individualized score for the user. For example, the DHP 406 may apply offset weights to each of the weights in the prediction model to compensate for each user's historical trends. Particularly, the DHP 406 is configured to recognize trends and correlations between decreased inhalation parameters, such as PIF and inhalation volume, and the onset of rescue inhaler usage events. As described herein, these trends may provide valuable insight into the state of the user's respiratory disease (e.g., the likelihood that the patient is to experience an exacerbation event) and/or the user's sensitivity to current or changing environmental conditions (e.g., heat, air quality, humidity, etc.). Further, the DHP 406 is configured to determine the severity of the rescue inhaler usage event based on the inhalation parameters captured during that particular event. As such, the DHP 406 is configured to generate individualized scores for users based on their historical inhalation parameters.

Based on the above referenced factors, the DHP 460 may conclude that a particular user is more (or less) sensitive to one environmental factor, such as humidity, than the average, and as such, their individualize future compliance and/or risk score would be increased when they are in a location having high humidity. For example, the DHP 406 may determine that a particular user tends to experience a decrease in one inhalation parameter, such as inhalation volume (e.g., as compared to the average of the dataset collected by the DHP 406 for that user and/or for the global population) when the user is in an environment with a condition, such as humidity, that exceeds a threshold, such as 70%. In response, the DHP 406 may include an offset weight to humidity when calculating the individualize future compliance and/or risk score for the user.

As a further example, the DHP 406 may be configured to recognize that another user tends to have reduced PIF values on their maintenance inhaler usage events prior to experiences a respiratory exacerbation. Accordingly, the DHP 406 is configured to add an offset weight to increase the weighting applied to “% of change in inhalation peak flow today, compared to last 3 days” when generating the user's individualized risk score.

Table 1 is one, non-limiting example, of the particular factors, or attributes, and associated weights that may be applied by the DHP 406 when determining a score (e.g., such as a user's individualized compliance score, a user's individualized future compliance score, and/or a user's individualized risk score). As noted herein, the weighting may be a biproduct of the machine learning algorithm, and for example, may indicate the relative significant of the particular factor/attribute when determining the particular score (the user's individualized compliance score, the user's individualized future compliance score, and/or the user's individualized risk score)

TABLE 1
Example Weighting Applied to Factors/Attributes
when determining an Individualized Score

| Factor/Attribute | | Weighting |
|---|---|---|
| Number of | Normalized* number of rescue | 0.104 |
| inhalations | inhalations (last 3 days) | |
| | Average number of daily rescue | 0.071 |
| | inhalations in the last 5 days | |
| | Missed number of maintenance | 0.069 |
| | events over the last 3 days | |
| | Normalized* number of rescue | 0.065 |
| | inhalations today | |
| | Normalized* number of | 0.056 |
| | inhalation events today | |
| | Maximal number of daily rescue | 0.051 |
| | inhalations in the last 5 days | |
| | Absolute number of rescue | 0.045 |
| | inhalations in the last | |
| | 3 days | |
| | Number of rescue inhalations | 0.042 |
| | 3 days ago | |
| | Number of rescue inhalations | 0.041 |
| | 4 days ago | |
| | Number of rescue inhalations | 0.026 |
| | 2 days ago | |
| | Absolute number of inhalation | 0.023 |
| | events today | |
| | % of change in number of rescue | 0.021 |
| | inhalations today, compared to | |
| | last 3 days | |
| | Number of rescue inhalations | 0.021 |
| | yesterday | |
| | Absolute number of rescue | 0.016 |
| | inhalations today | |
| | Absolute number of rescue | 0.008 |
| | inhalations during night time | |
| | in the last 3 days | |
| | Total weighting: number of | 0.659 |
| | inhalations | |
| Inhalation | % of change in inhalation peak | 0.082 |
| parameters | flow today, compared to last | |
| | 3 days | |
| | % of change in inhalation | 0.051 |
| | volume today, compared to | |
| | last 3 days | |
| | Normalized* inhalation peak | 0.046 |
| | flow today | |
| | Normalized* inhalation | 0.037 |
| | volume today | |
| | Total weighting: inhalation | 0.216 |
| | parameters | |
| Biometric | Exacerbations and medical | 0.012 |
| parameter | history | |
| | Body mass index | 0.009 |
| | Systolic blood pressure | 0.005 |
| | Total weighting: biometric | 0.026 |
| | parameter | |
| Environmental | Humidity | 0.039 |
| Conditions | Ozone | 0.031 |
| | Particulate Matter | 0.005 |
| | Temperature | 0.014 |
| Daily SA | Self-Assessment Responses | 0.011 |

*The term “normalized” means relative to the respective baseline

The DHP 406 may send an alert to a user device associated with the user upon determining that the user is likely to experience an exacerbation, such as when the user's individualized risk score is above a threshold (e.g., individualized risk scores greater than 7) that indicates that the user is at a high likelihood of a respiratory exacerbation. The user device may generate the alert and/or send a message to one or more of the user's inhalers that cause the inhalers to generate the alert for the user. The alert could be any one or more of an audible noise generated by a speaker, an illuminate light generated by a light source, a GUI presented on the user device, etc. The alert may indicate that the user should keep their rescue inhaler nearby. Further, the threshold may, for example, be a predetermined value that is compared to an output by the machine learning algorithm, and that signifies a high probability that the user will have difficulty breathing and/or feel the need to use their respiratory rescue medication in the immediate future (e.g., within the next X hours, such as 12 hours, or days, such as 1 day).

Similarly, based on the DHP 406 determining that a particular user is particularly sensitive to a specific environmental condition (e.g., based on the user having decreased inhalation parameters for usage events associated with the specific environmental condition), the DHP 406 may send an alert to the user based on one or more environmental conditions associated with a present location of the user. As noted above, the DHP 406 may determine one or more environmental conditions associated with an inhalation parameter of a usage event using the time and geographic location associated with the usage event. Based on the correlation between the inhalation parameter and the environmental condition (e.g., such as 20% lower PIF when the usage event occurs in humidity over 70%), the DHP 406 may send an alert to a user device associated with the user based on a determination that one or more environmental conditions associated with a present location of the user are above a threshold. The threshold may be user specific (e.g., different for different users), for example, based on the user's general sensitivity to that particular environmental condition.

The DHP 406 may generate an individualized compliance score for a user. The individualized compliance score for the user may indicate how compliance the user has been when taking their medication. For example, the compliance score may indicate how well the user has inhaled via their one or more inhalers—did the user inhale deep enough (e.g., based on PIF), long enough (e.g., based on inhaled volume), etc. Further, in some examples, the compliance score may also include an indication of the user's adherence to their prescribed dosing schedule (e.g., in the situations where the user is prescribed one or more inhalers with a maintenance medicament). The DHP 406 may generate the compliance score using one or more of the machine learning algorithms described herein, such as an unsupervised model. In some examples, a higher compliance score may indicate that the user has been more compliant with their use of their inhalers, while a lower compliance score may indicate that the user has been less compliance with their use of their inhalers.

The DHP 460 may generate a future compliance score for a user. The individualized future compliance score for the user may indicate how compliant the user has been when taking their medication (e.g., did the user inhale deep enough (e.g., based on PIF), long enough (e.g., based on inhaled volume), etc.). Further, in some examples, the future compliance score may also include a prediction of how adherent the user will be with respect to their prescribed dosing schedule (e.g., in the situations where the user is prescribed one or more inhalers with a maintenance medicament). The DHP 406 may generate the future compliance score using one or more of the machine learning algorithms described herein, such as an unsupervised or supervised model. In some examples, a higher future compliance score may indicate that the user is more likely to be compliant with their use of their inhalers going forward, while a lower compliance score may indicate a prediction that the user will be less compliance with their use of their inhalers going forward.

The DHP 406 may generate one or more suggestions for improving the user's future compliance score. The DHP 406 may determine an attribute that the user should improve upon, for example, to improve their future compliance score. For example, the DHP 406 may use the trained machine learning algorithm to determine the attribute to improve. The attribute may include taking a maintenance medicament at a different time of day, increasing the PIF of future usage events, and/or increasing inhaled volume of future usage events. The DHP 406 may determine a significance factor for each attribute. The DHP 406 may determine the attribute to improve based on the significance factor(s). The significance factor(s) may be based on the weight of each attribute in the machine learning algorithm's determination of the user's future compliance score, and for example, the individual user's attribute (e.g., PIF) based on an average of that attribute for all user's within the training dataset (e.g., the user's relative PIF as compared to the PIF of the other usage event's that make up the training dataset for the machine learning model). The DHP 406 may suggest that the attribute with the highest significance should be improved by the user to improve compliance. Additionally or alternatively, the DHP 406 may indicate a ranked list of attributes to the user in a notification (e.g., via a display device), for example, so that the user and/or the user's health care provider can decide which attribute(s) the user should attempt to improve upon.

The DHP 406 and/or the user device 402a, 402b may use the individualized compliance score, individualized future compliance score, and/or individualized risk score determined by the DHP 406 and the currently environmental conditions in a space to control or adjust certain smart devices or appliances (e.g., devices or appliances with processing and communication capabilities) that may contribute to the patient's likelihood of experiencing an exacerbation. For example, the DHP 460 and/or the user device 402a, 402b may be configured to control one or more smart devices installed at the present location of the, such as a smart heating ventilation and air conditioning (HVAC) system residing at the user's home. Further, in addition to the individualized compliance score, individualized future compliance score, and/or the individualized risk score, control and/or adjustment of smart devices or appliances that contribute to a patient's likelihood of experiencing an exacerbation may also consider the location of the user and/or past, current, or future weather conditions at that location.

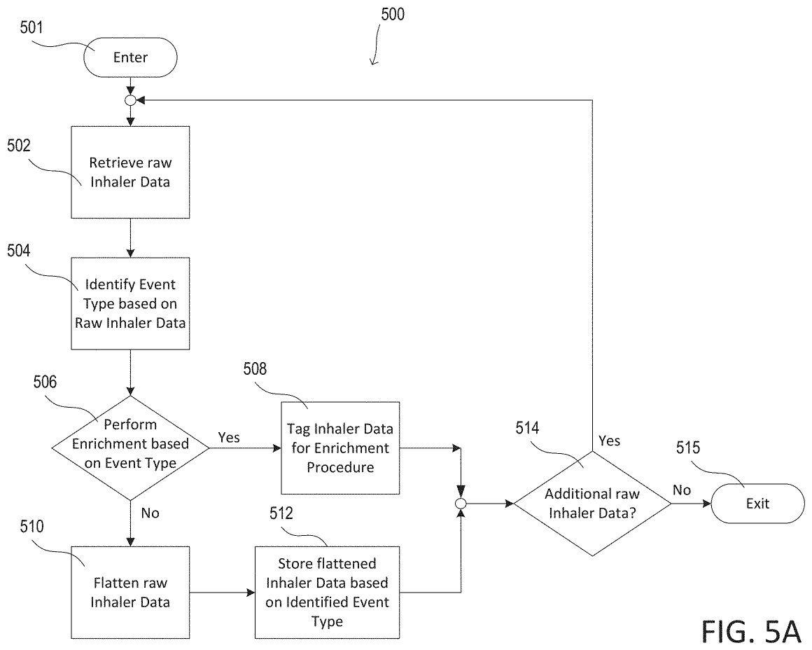

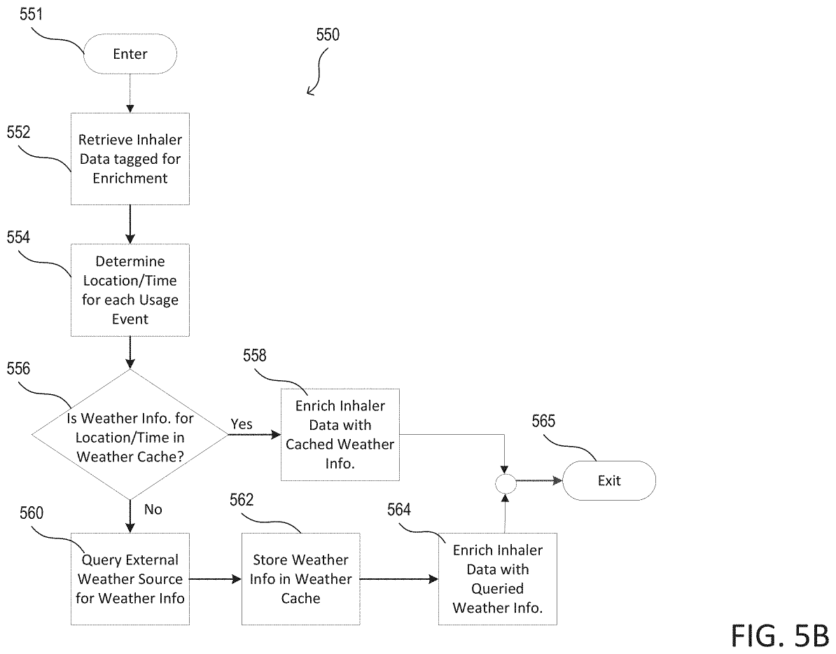

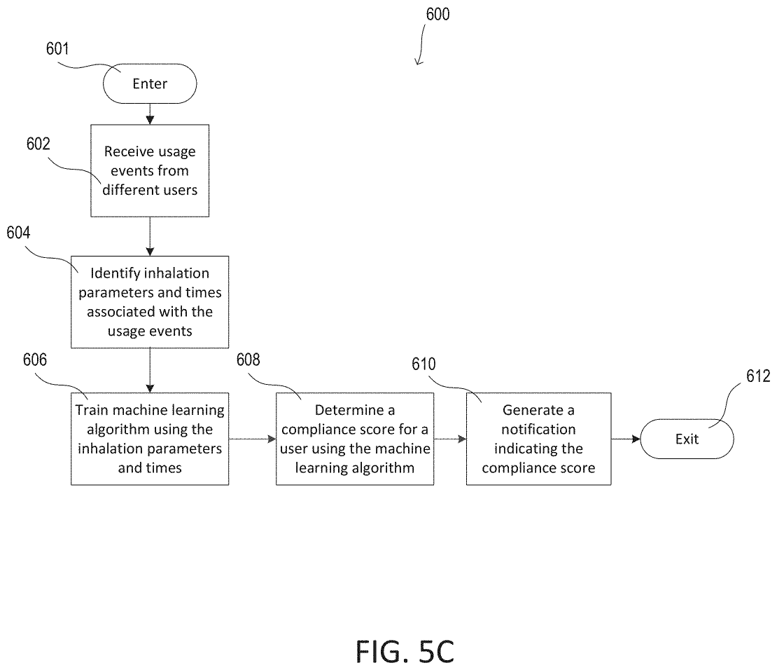

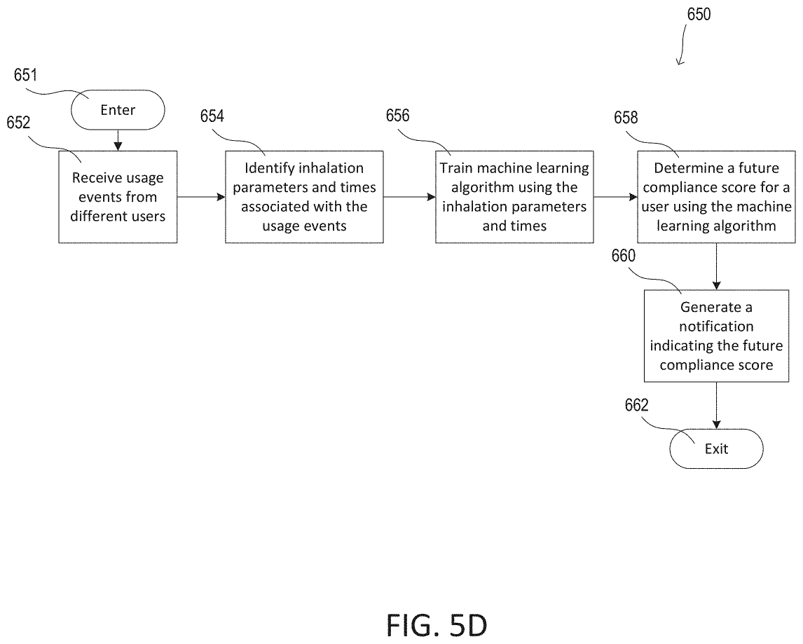

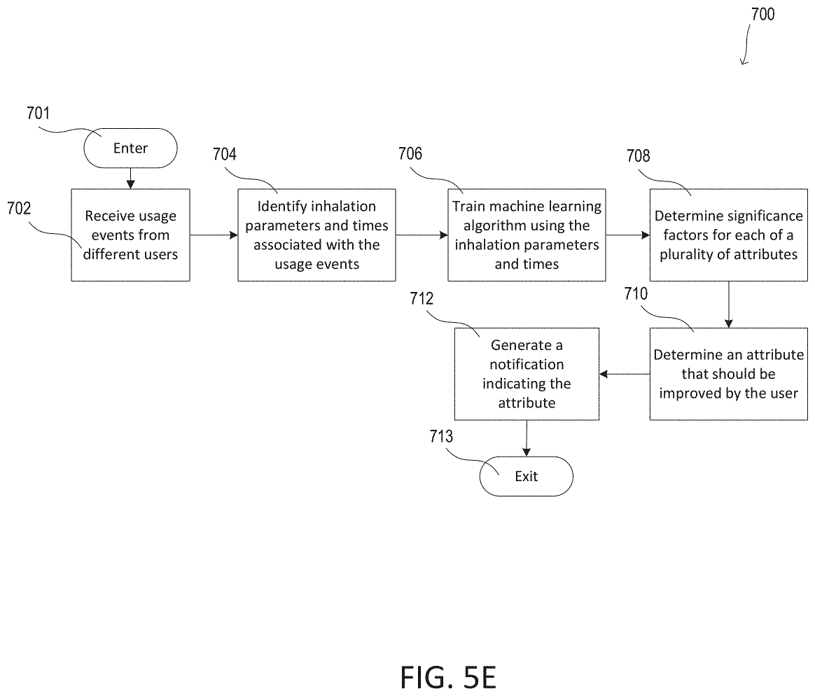

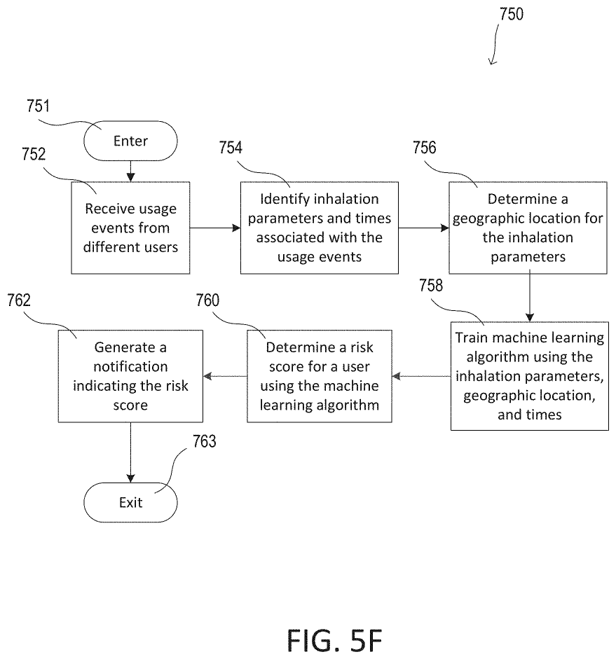

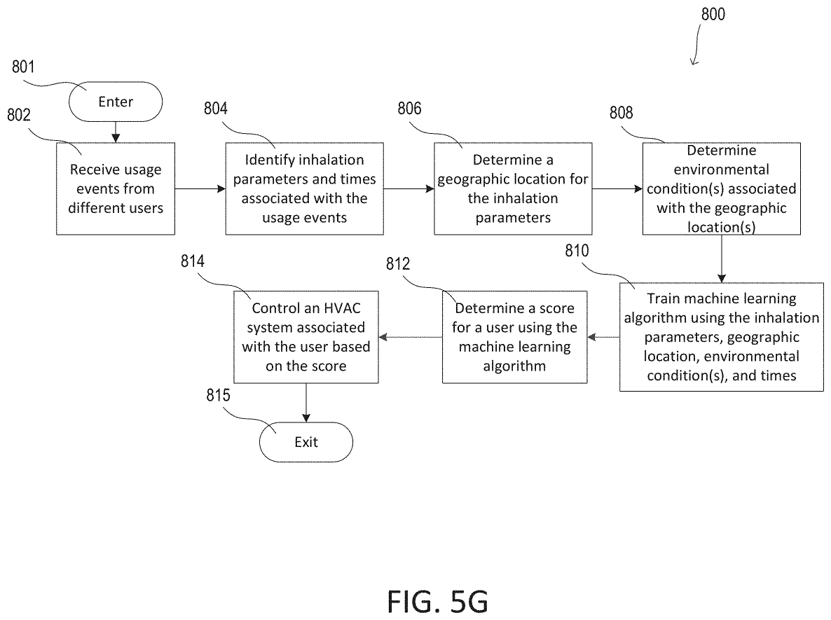

Referring to a smart HVAC unit, for example, the DHP 460 and/or the user device 402a, 402b may control or adjust the humidity and/or temperature levels of the smart HVAC system, for example, as illustrated in FIG. 5E. For example, the DHP 406 may retrieve the current humidity and temperature level of the space that the smart HVAC resides. Then, based on the individualized score for the user, the user's location, and/or past, current, or future weather conditions at that location, the DHP 460 and/or the user device 402a, 402b may control or adjust to set humidity and/or temperature of the HVAC system to decrease the likelihood that the patient experiences an exacerbation event. For example, humidity levels greater than 80% and/or temperature levels greater than 75 degrees may, in some situations, increase the likelihood of an exacerbation event. And, for example, when a patient's individualized score already indicates an increased likelihood that the patient will experience an exacerbation event when exposed to high humidity and/or high temperature, the DHP 460 and/or the user device 402a, 402b may control the HVAC system to adjust the internal humidity level below 80% and/or the temperature level below 75 degrees.

Further, in some instances, the DHP 460 and/or the user device 402a, 402b may determine the location of the user (e.g., based on the location of the user device), and coordinate the adjustment of the HVAC system to occur before the user reaches the location. For example, the DHP 460 and/or the user device 402a, 402b may control the HVAC system to adjust the set humidity and/or temperature when the user is determined to be a predetermined distance from the location.

As described herein, the DHP 406 may enrich or relate the inhaler data it receives from one or more external sources. FIG. 5A illustrates an example procedure 500 for enriching inhaler data. For example, the procedure 500 may use an extraction, transformation, loading (ETL) external weather source. The procedure 500 may be performed by the DHP 406. The procedure 500 may be performed periodically, for example, in response to alarm or schedule that may be configurable. For example, the procedure 500 may be performed on a periodic basis (e.g., performed every 15 minutes). Also, or alternatively, the frequency at which the procedure 500 is performed may vary, for example, in response to amount of inhaler data received by the DHP 406 (e.g., when the amount of inhaler data received by the DHP 406 increases). As illustrated in FIG. 5A, the procedure 500 may enter at 501.

At 502, the DHP 406 may retrieve a raw record of an instance of received inhaler data. For the example, the retrieved raw inhaler data may be the next received instance of inhaler data after a last processed raw record in received inhaler data. A raw record of an instance of received inhaler data may include the raw information or data (e.g., in a certain format, such as JavaScript object notation (JSON) format) transmitted to the DHP 406 from a user device, as described herein. After retrieving the raw inhaler data, the DHP 406 may parse the raw inhaler data to identify an event type at 504. For example, the identified event type may comprise one of self-assessment (e.g., a daily self-assessment provided by the user, for example, using the End-User or Patient facing processing module of a user device, as described herein), a usage event (e.g., inhalation of an inhaler), a classification assigned to a given usage event (e.g., Low inhalation, Fair inhalation, Good inhalation, Exhalation, and Vent Block event types), etc. Also, or alternatively, the identified event type may include event types detected by one or more third party applications running on the user device (e.g., Apple Health app) or another user device (e.g., FitBit, Apple Watch, etc.). At 506, the DHP may determine whether to initiate or perform an enrichment procedure (e.g., as described in further detail with respect to FIGS. 5A and 5B) based on the event type identified at 504. As described herein, an enrichment procedure may be used to enrich or relate inhaler data with certain environmental information (e.g., time, geographic location, weather conditions, health related data, etc.). For example, if the event type is identified to be any one of a self-assessment or a usage event, the DHP 406 may determine to tag the inhaler data for an enrichment procedure at 508 (e.g., indicate that the inhaler data should be enriched with an enrichment procedure). The DHP 406 may also indicate a time at which to initiate the enrichment procedure, for example. For example, the DHP 406 may indicate that a certain enrichment procedures (e.g., weather observation enrichment) may be performed at least 12 hours after a time associated with the inhaler data (e.g., the time that an inhalation or self-assessment occurred). Similarly, the DHP 406 may indicate that other types enrichment procedures (e.g. air quality enrichment and/or pollen enrichment) may be performed at least 24 hours after a time associated with the inhaler data (e.g., the time that an inhalation or self-assessment occurred). As further described herein, an enrichment procedure may enrich or add inhaler data with relevant information from one or more external sources.

If, however, the DHP determines not to tag the inhaler data for an enrichment procedure, the DHP may flatten the raw inhaler data at 510 and store the flattened data at 512. For example, flattening and storing the raw inhaler data may allow the inhaler data to be stored at a single location (e.g., single database or table), which may increase the efficiency or speed at which the inhaler data may be retrieved or otherwise manipulated and/or the efficient or speed at which analytics may be performed on the inhaler data. At 514, the DHP may determine whether there is additional raw inhaler data to perform the ETL procedure 500 on. If, for example, the DHP determines that there is no more additional inhaler data on which to perform the ETL procedure 500, the procedure 500 may exit at 515. If, however, the DHP determines that are additional instances of inhaler data on which to perform the ETL procedure, the procedure 500 may retrieve a next subsequent instance of raw inhaler data.

FIG. 5B illustrates an example enrichment procedure 550 for enriching inhaler data with data from an external source, such as an external weather source. The procedure 550 may be performed periodically, for example, every 24 hours, which may allow for the information received from the external sources to stabilize. As illustrated in FIG. 5B, the procedure 550 may be enter at 551. At 552, the DHP may retrieve the inhaler data that is tagged for an enrichment procedure. For example, the inhaler data may be tagged for an enrichment procedure during a performance of the procedure 500 (e.g., at 508) based on the identified event type of the inhaler data. Further, as described herein, the inhaler data may also indicate a time at which to perform the enrichment procedure, and the inhaler data retrieved at 552 may only include the inhaler data that is ripe (e.g., the identified minimum amount time has passed since the time that the inhaler data was originated) for enrichment. The DHP may retrieve this information from the operational subsystem 406a (e.g., after the respective user devices transmit the inhaler data to DHP using a communication interface published by the DHP, as described herein). At 554, the DHP may determine the location and time for each of the retrieved usage events, which, again may be indicated in the inhaler data or otherwise stored in the operational subsystem 406a.

At 556, the DHP may determine whether weather information for the location and time of each usage event is stored within a weather cache, which as described herein, may be maintained by the DHP. For example, the weather information may include certain information or data (e.g., weather observations, pollen reports, air quality, etc.) related to the weather conditions present that time and location. If the weather information for the location and time of a respective usage event is stored within the weather cache, the DHP may enrich the inhaler data or usage event with the cached weather information for that location and/or time at 558. If, however, the respective weather information is not stored within the weather cache, the DHP may query the external weather source for the relevant weather information at 560. After querying for the weather information, the DHP may store the weather information in the weather cache at 562, and enrich the inhaler data or usage event with the queried weather information at 564. The procedure 550 may exit at 565, for example, following enriching inhaler data with cached or queried weather information.

As described herein, the DHP 406 may train a machine learning algorithm using data from inhalers 401a-d (e.g., via a respectively paired user device), which may be associated with a plurality of different users. For example, the DHP 406 may receive the data from respective user devices (e.g., such as the user devices 402a-d shown in FIGS. 4A and 4B) and/or respective inhalers (e.g., such as the inhalers 401a-d shown in FIGS. 4A and 4B) associated with the users. The DHP 406 may determine a compliance score for a user using the machine learning algorithm. FIG. 5C illustrates an example procedure 600 for determining a compliance score for a user. The procedure 600 may be performed by the DHP 406, for example, the analytical subsystem 406a and/or the operational subsystem 406b. The procedure 600 may be performed periodically, for example, in response to alarm or schedule that may be configurable. For example, the procedure 600 may be performed on a periodic basis (e.g., performed every 15 minutes). Additionally or alternatively, the frequency at which the procedure 600 is performed may vary, for example, in response to amount of inhaler data received by the DHP 406 (e.g., when the amount of inhaler data received by the DHP 406 increases). As illustrated in FIG. 5C, the procedure 600 may enter at 601.

At 602, the DHP 406 may receive usage events associated with different users. Each usage event may be associated with a medicament type and a user of the different users. The usage events may include maintenance usage events associated with a maintenance medicament type and/or rescue usage events associated with a rescue medicament type. The maintenance usage events may be associated with a dosing schedule for the maintenance medicament type. Each usage event may include a time (e.g., a relative time or an absolute time) associated with the usage event and one or more inhalation parameters of the usage event. For example, the DHP 406 may retrieve raw records of specific instances of received inhaler data. A raw record of a specific instance of received inhaler data may include the raw information or data (e.g., in a certain format, such as JavaScript object notation (JSON) format) transmitted to the DHP 406 from a user device, as described herein. The raw information or data may include the time, inhalation parameters, etc.

The DHP 406 may anonymize the raw information or data for the usage events. For example, the DHP 406 may perform a one-way hash (e.g., apply a one-way hash function) on the usage events to anatomize the usage event data.

At 604, the DHP 406 may identify the times and inhalation parameters of the usage events. After retrieving the raw inhaler data, the DHP 406 may parse the raw inhaler data to identity the times and inhalation parameters at 504. The inhalation parameters may include one or more of a PIF, an inhalation volume, a time to peak inhalation flow, an inhalation duration, etc.

At 606, the DHP 406 may train a machine learning algorithm. The DHP 406 may train, at 406, the machine learning algorithm using training data. The training data may include a plurality of usage events (e.g., medicament type, time, inhalation parameters, geographic data, environmental factors, and/or any of the attributes/factors described herein), where each usage event may be associated with one or a plurality of different users. The training data may include compliance data, adherence data, and/or self-assessment response data. For example, the training data may include one or more of the times associated with the usage events, the inhalation parameters associated with the usage events, an adherence ratio, a number of rescue usage events in a predetermined number of days, a number of missed maintenance usage events in the predetermined number of days, a frequency of rescue usage events, a percent change in PIF, or a percent change in inhalation volume. The adherence ratio may indicate a user's adherence to a dosing schedule, for example, for a maintenance medicament type. For example, the adherence ratio may be determined based on a comparison between a number of maintenance usage events of the user over a predetermined period of time and a number of maintenance usage events indicated by the dosing schedule for the predetermined period of time. The predetermined period of time may be a predetermined number of days.

Additionally or alternatively, the training data may include data received via a self-assessment (e.g., a daily self-assessment provided by the user, for example, using the End-User or Patient facing processing module of a user device, as described herein), one or more third party applications running on the user device (e.g., Apple Health app), or another user device (e.g., FitBit, Apple Watch, etc.). The machine learning algorithm may be trained, at 606, continuously (e.g., hourly, daily, weekly, etc.). For example, additional data received (e.g., after initial training) may be used to train the machine learning algorithm.

The machine learning algorithm may be trained, at 606, using an unsupervised learning method. The unsupervised learning method may be a type of machine learning that does not use labels for training data. The unsupervised learning method may be a learning method for learning artificial neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters. Although described with reference to an unsupervised learning method, in some examples, the DHP 406 may train the machine learning using a supervised learning method.

At 608, the DHP 406 may determine a compliance score for a user using the machine learning algorithm. The compliance score may be measure of the user's technique when using a particular drug delivery device (e.g., did the user inhaler deep enough (e.g., based on PIF), long enough (e.g., based on inhaled volume), at the best time(s) of day/night, etc.). For example, the compliance score may indicate how compliant the user has been during usage events over a period of time. The period of time may comprise a predetermined (e.g., last predetermined) number of days. If the patient is using the device in a manner that is recommended by a doctor or by a manufacturer, the device is likely to deliver the desired dose of medication and the compliance score for the user may be determined to be at a high level. However, if the device is not being used properly during drug administration, the device's ability to deliver a proper dose of medication may be compromised and the compliance score for the user may be determined to be at a low level. The compliance score may be a measure of the effectiveness of a prescribed treatment for the user over the period of time.

When the training data is anonymized, the operational subsystem 406a of the DHP 406 may provide a user's data to the analytics subsystem 406b (e.g., anonymized), and the analytics subsystem 460b may determine a compliance score based on the anonymized data, and return the score to the operational subsystem 406a. The operational subsystem 406a may then associate the compliance score with the user.

At 610, the DHP 406 may generate a notification indicating the compliance score. The notification may be displayed for a practitioner and/or health care professional, for example, to allow them to analyze the user's compliance with the prescribed treatment. In one example, the DHP 406 causes the computer 408 associated with the health care provider to provide the compliance score and/or inhalation data via a graphical user interface (GUI) that is presented on the health care provider's computer. Additionally or alternatively, the notification may be displayed to the user via a display device (e.g., such as user devices 402a, 402b shown in FIG. 4A and/or user devices 402c, 402d shown in FIG. 4B). A patient using an inhaler may not be able to correct a non-compliant use because he or she may not be able to immediately detect or sense that medication is being inhaled and/or know whether the amount of inhaled medication complies with the prescription. Thus, the notification indicating the compliance score may provide feedback to the user of whether the user is complying with prescribed treatment. The procedure 600 may exit at 612, for example, following generation and/or sending the notification indicating the compliance score.

Further, in some examples, the DHP 406 may determine an attribute that the user should improve upon, for example, to improve their compliance score. For example, the DHP 406 may use the trained machine learning algorithm to determine the attribute to improve. The DHP 406 may include the attribute in the notification generated at 610. The attribute may include taking a maintenance medicament at a different time of day, increasing the PIF of future usage events, and/or increasing inhaled volume of future usage events. The DHP 406 may determine a significance factor for each attribute. The DHP 406 may determine the attribute to improve based on the significance factor(s). The significance factor(s) may indicate the weight of each attribute in the user's compliance (e.g., compliance score). The DHP 406 may suggest to the user that the attribute with the highest significance (e.g., significance factor) should be improved by the user to improve compliance.

As described herein, the DHP 406 may determine a future compliance score for a user using the machine learning algorithm. FIG. 5D illustrates an example procedure 650 for determining a future compliance score for a user. The procedure 650 may be performed by the DHP 406, for example, the analytical subsystem 406a and/or the operational subsystem 406b. The procedure 650 may be performed periodically, for example, in response to alarm or schedule that may be configurable. For example, the procedure 650 may be performed on a periodic basis (e.g., performed every 15 minutes). Additionally or alternatively, the frequency at which the procedure 650 is performed may vary, for example, in response to amount of inhaler data received by the DHP 406 (e.g., when the amount of inhaler data received by the DHP 406 increases). As illustrated in FIG. 5D, the procedure 650 may enter at 651.

At 652, the DHP 406 may receive usage events associated with different users. The DHP 406 may receive the usage events from respective user devices (e.g., such as user devices 402a-d shown in FIGS. 4A and 4B) and/or inhalers 401a-401d. Each usage event may be associated with a medicament type, an inhaler, and a user of the different users. The usage events may include maintenance usage events associated with a maintenance medicament type and rescue usage events associated with a rescue medicament type. The maintenance usage events may be associated with a dosing schedule for the maintenance medicament type. Each usage event may include a time associated with the usage event and one or more inhalation parameters of the usage event. For example, the DHP 406 may retrieve raw records of specific instances of received inhaler data. A raw record of a specific instance of received inhaler data may include the raw information or data (e.g., in a certain format, such as JavaScript object notation (JSON) format) transmitted to the DHP 406 from a user device, as described herein. The raw information or data may include the time, inhalation parameters, etc.

The DHP 406 may anonymize the raw information or data for the usage events. For example, the DHP 406 may perform a one-way hash (e.g., apply a one-way hash function) on the usage events to anatomize the usage event data.

At 654, the DHP 406 may identify the times and inhalation parameters associated with the usage events. After retrieving the raw inhaler data, the DHP 406 may parse the raw inhaler data to identify the times and inhalation parameters at 654. The inhalation parameters may include one or more of a PIF, an inhalation volume, a time to peak inhalation flow, an inhalation duration, etc.

At 656, the DHP 406 may train a machine learning algorithm. The DHP 406 may train, at 406, the machine learning algorithm using training data. The training data may include a plurality of usage events (e.g., medicament type, time, inhalation parameters, geographic data, environmental factors, and/or any of the attributes/factors described herein), where each usage event may be associated with one or a plurality of different users. The training data may include compliance data, adherence data, and/or self-assessment response data. For example, the training data may include one or more of the times associated with the usage events, the inhalation parameters associated with the usage events, an adherence ratio, a number of rescue usage events in a predetermined number of days, a number of missed maintenance usage events in the predetermined number of days, a frequency of rescue usage events, a percent change in PIF, or a percent change in inhalation volume. The frequency of rescue usage events may be an average number of daily rescue usage events for the user over a predetermined period of time or an absolute number of daily rescue usage events for the user over the predetermined period of time. The predetermined period of time may be a predetermined number of days. The predetermined number of days may be a predetermined number of last days or days prior. The adherence ratio may indicate a user's adherence to a dosing schedule, for example, for a maintenance medicament type. For example, the adherence ratio may be determined based on a comparison between a number of maintenance usage events of the user over a predetermined period of time and a number of maintenance usage events indicated by the dosing schedule for the predetermined period of time.

Additionally or alternatively, the training data may include data received via a self-assessment (e.g., a daily self-assessment provided by the user, for example, using the End-User or Patient facing processing module of a user device, as described herein), one or more third party applications running on the user device (e.g., Apple Health app), or another user device (e.g., FitBit, Apple Watch, etc.). The machine learning algorithm may be trained, at 656, continuously (e.g., hourly, daily, weekly, etc.). For example, additional data received (e.g., after initial training) may be used to train the machine learning algorithm.

The machine learning algorithm may be trained, at 656, using an unsupervised learning method or a supervised learning method. An unsupervised learning method may be a type of machine learning that does not use labels for training data. The unsupervised learning method may be a learning method for learning artificial neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters.

A supervised learning method may use labeled training data to train the machine learning algorithm. As training data is received, the supervised learning method may adjust weights until the machine learning algorithm is appropriately weighted. The supervised learning method may measure the accuracy of the machine learning algorithm using a loss function. The supervised learning method may continue adjusting the weights until the error is reduced below a predetermined threshold. The supervised learning method may comprise gradient boosted decision trees. Gradient boosted decision trees may combine weak learners to minimize the loss function. For example, regression trees may be sued to output real values for splits and to be added together. The weak learners may be constrained, for example, to a maximum number of layers, a maximum number of nodes, a maximum number of splits, etc. Trees may be added one at a time to the machine learning algorithm and existing trees may remain unchanged. A gradient descent procedure may be used to minimize loss when adding trees. For example, additional trees may be added to reduce the loss (e.g., follow the gradient). In this case, the additional tree(s) may be parameterized and those parameters may be modified to reduce the loss. The supervised learning method may comprise an XGBoost algorithm. The XGBoost algorithm may comprise an implementation of gradient boosted decision trees that is designed for speed and/or performance. The XGBoost algorithm may automatically handle missing data values, support parallelization of tree construction, and/or continued training.

In some examples, the DHP 406 may determine a geographic location for the inhalation parameters of each usage event. For example, the DHP 406 may receive location data associated with the inhalers (e.g., such as inhalers 401a-401d shown in FIG. 4A) and/or user devices (e.g., such as user devices 402a-402d shown in FIGS. 4A and 4B). The DHP 406 may tag each of the inhalation parameters with the determined geographic location. The DHP 406 may determine a point of interest based on the geographic location. The point of interest may include a park, a fuel station, a factory, a power plant, a highway, a user's home, a user's workplace, a train station, etc. The point of interest may be associated with the inhalation parameters. The point of interest(s) may be used to classify the inhalation parameters and/or train the machine learning algorithm. In examples, the geographic location may be identified using latitude and longitude coordinates, a geohash, a mapcode, or another type of geocoding system. The point of interest may comprise an area having a range of geographic locations (e.g., latitude and longitude coordinates, geohashes, mapcodes, etc.).

In some examples, the DHP 406 may determine one or more environmental conditions for each usage event, for example, using the respective time and geographic location associated with each usage event. The environmental condition(s) may be included in the training data and may be used to train the machine learning algorithm. The environmental condition(s) may include one or more of temperature, humidity, outdoor air pollutant concentration, presence of particulate matter of 2.5 microns or smaller (PM2.5), presence of particulate matter of 10 microns or smaller (PM10), ozone concentration, nitrogen dioxide (NO₂) concentration, or sulfur dioxide (SO₂) concentration.

At 658, the DHP 406 may determine a future compliance score for a user using the machine learning algorithm. The future compliance score may indicate how compliant the user will be moving forward. For example, the future compliance score may indicate a risk that the user will be non-compliant in future usage events. In examples, the future compliance score may indicate a probability that the user's use of an inhalation device will comply with the inhalation device specifications. The future compliance score may be determined using pattern detection. For example, the machine learning algorithm may compare a pattern associated with the user to one or more patterns associated with a plurality of users. The pattern associated with the user may be determined using the user's training data over a previous number of days and the one or more patterns associated with the plurality of users may include the plurality of user's training data. The pattern may include one or more maintenance usage events, inhalation parameters associated with one or more maintenance usage events, one or more rescue usage events, inhalation parameters associated with one or more rescue usage events, and/or adherence to a dosing schedule over a period of time. The period of time may comprise a predetermined (e.g., last predetermined) number of days. The future compliance score may be determined, at 658, using the geographic location and/or point of interest where the user is located. For example, the DHP 406 may identify a pattern associated with the user's geographic location and/or point of interest and may adjust the future compliance score accordingly.

When the training data is anonymized, the operational subsystem 406a of the DHP 406 may provide a user's data to the analytics subsystem 406b (e.g., anonymized), and the analytics subsystem 460b may determine a future compliance score based on the anonymized data, and return the future compliance score to the operational subsystem 406a. The operational subsystem 406a may then associate the future compliance score with the user.

At 660, the DHP 406 may generate a notification indicating the future compliance score. The notification may be displayed for a practitioner and/or health care professional, for example, to allow them to analyze the user's expected compliance with future treatment. In one example, the DHP 406 causes the computer 408 associated with the health care provider to provide the future compliance score and/or inhalation data via a graphical user interface (GUI) that is presented on the health care provider's computer. Additionally or alternatively, the notification may be displayed to the user via a display device (e.g., such as user devices 402a, 402b shown in FIG. 4A and/or user devices 402c, 402d shown in FIG. 4B). A patient using an inhaler may not be able to correct a non-compliant use of the inhaler because he or she may not be able to detect (e.g., immediately detect) or sense that medication is being inhaled and/or know whether the amount of inhaled medication complies with the prescribed treatment. Thus, the notification indicating the future compliance score may provide feedback to the user of whether the user is likely to comply with future treatment (e.g., scheduled or unscheduled treatment).

In some examples, the DHP 406 may determine an attribute that the user should improve upon, for example, to improve their future compliance score. For example, the DHP 406 may use the trained machine learning algorithm to determine the attribute to improve. The DHP 406 may include the attribute in the notification generated at 660. The attribute may include taking a maintenance medicament at a different time of day, increasing the PIF of future usage events, and/or increasing inhaled volume of future usage events. The DHP 406 may determine a significance factor for each attribute. The DHP 406 may determine the attribute to improve based on the significance factor(s). The significance factor(s) may indicate the weight of each attribute in the user's compliance (e.g., compliance score). The DHP 406 may suggest that the attribute with the highest significance (e.g., significance factor) should be improved by the user to improve compliance. The procedure 650 may exit at 662, for example, following generation and/or sending the notification indicating the future compliance score.

As described herein, the DHP 406 may train a machine learning algorithm using data from inhalers 401a-d (e.g., via a respectively paired user device), which may be associated with a plurality of different users. The DHP 406 may determine one or more attributes that a user should/could improve upon using the machine learning algorithm. FIG. 5E illustrates an example procedure 700 for determining an attribute that should be improved by a user. The procedure 700 may be performed by the DHP 406, for example, the analytical subsystem 406a and/or the operational subsystem 406b. The procedure 700 may be performed periodically, for example, in response to alarm or schedule that may be configurable. For example, the procedure 700 may be performed on a periodic basis (e.g., performed every 15 minutes). Additionally or alternatively, the frequency at which the procedure 700 is performed may vary, for example, in response to amount of inhaler data received by the DHP 406 (e.g., when the amount of inhaler data received by the DHP 406 increases). As illustrated in FIG. 5D, the procedure 700 may enter at 701.

At 702, the DHP 406 may receive usage events associated with different users. Each usage event may be associated with a medicament type and a user of the different users. The usage events may include maintenance usage events associated with a maintenance medicament type and rescue usage events associated with a rescue medicament type. The maintenance usage events may be associated with a dosing schedule for the maintenance medicament type. Each usage event may include a time associated with the usage event and one or more inhalation parameters of the usage event. For example, the DHP 406 may retrieve raw records of specific instances of received inhaler data. A raw record of a specific instance of received inhaler data may include the raw information or data (e.g., in a certain format, such as JavaScript object notation (JSON) format) transmitted to the DHP 406 from a user device, as described herein. The raw information or data may include the time, inhalation parameters, etc.

The DHP 406 may anonymize the raw information or data for the usage events. For example, the DHP 406 may perform a one-way hash (e.g., apply a one-way hash function) on the usage events to anatomize the usage event data.

At 704, the DHP 406 may identify the times and inhalation parameters associated with the usage events. After retrieving the raw inhaler data, the DHP 406 may parse the raw inhaler data to identity the times and inhalation parameters at 704. The inhalation parameters may include one or more of a PIF, an inhalation volume, a time to peak inhalation flow, an inhalation duration, etc.

At 706, the DHP 406 may train a machine learning algorithm. The DHP 406 may train, at 406, the machine learning algorithm using training data. The training data may include a plurality of usage events (e.g., medicament type, time, inhalation parameters, geographic data, environmental factors, and/or any of the attributes/factors described herein), where each usage event may be associated with one or a plurality of different users. The training data may include compliance data, adherence data, and/or self-assessment response data. For example, the training data may include one or more of the times associated with the usage events, the inhalation parameters associated with the usage events, an adherence ratio, a number of rescue usage events in a predetermined number of days, a number of missed maintenance usage events in the predetermined number of days, a frequency of rescue usage events, a percent change in PIF, or a percent change in inhalation volume. The adherence ratio may indicate a user's adherence to a dosing schedule, for example, for a maintenance medicament type. For example, the adherence ratio may be determined based on a comparison between a number of maintenance usage events of the user over a predetermined period of time and a number of maintenance usage events indicated by the dosing schedule for the predetermined period of time. The predetermined period of time may be a predetermined number of days. The machine learning algorithm may be trained, at 706, continuously (e.g., hourly, daily, weekly, etc.). For example, additional data received (e.g., after initial training) may be used to train the machine learning algorithm.

The machine learning algorithm may be trained, at 706, using an unsupervised learning method. The unsupervised learning method may be a type of machine learning that does not use labels for training data. The unsupervised learning method may be a learning method for learning artificial neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters. Although described with reference to an unsupervised learning method, in some examples, the DHP 406 may train the machine learning algorithm using a supervised learning method.

At 708, the DHP 406 may determine a significance factor for each of a plurality of attributes. The plurality of attributes may include taking a maintenance medicament at a different time of day, increasing the PIF of future usage events, and/or increasing inhaled volume of future usage events. The significance factor may indicate a significance of the respective attribute. For example, the significance factor for an attribute may indicate a relative importance of that attribute.

At 710, the DHP 406 may determine an attribute that the user should improve upon, for example, to improve their compliance score and/or future compliance score. For example, the DHP 406 may use the trained machine learning algorithm and/or the determined significance factor(s) to determine the attribute to improve. The significance factor(s) may indicate the relative weight of each attribute in the user's compliance (e.g., compliance score). The DHP 406 may suggest that the attribute with the highest significance (e.g., significance factor) should be improved by the user to improve compliance.

At 712, the DHP 406 may generate a notification indicating the determined attribute. The notification may be displayed for a practitioner and/or health care professional, for example, to allow them to provide feedback to the user and/or alter the prescribed treatment. In one example, the DHP 406 causes the computer 408 associated with the health care provider to provide the attribute with a compliance score, a future compliance score, and/or inhalation data via a graphical user interface (GUI) that is presented on the health care provider's computer. Additionally or alternatively, the notification may be displayed to the user via a display device (e.g., such as user devices 402a, 402b shown in FIG. 4A and/or user devices 402c, 402d shown in FIG. 4B). The notification displayed to the user indicating the attribute may provide feedback to the user of how the user can improve compliance with and/or adherence to a prescribed treatment. The procedure 700 may exit at 713, for example, following generation and/or sending the notification indicating the attribute.

As described herein, the DHP 406 may determine a risk score for a user using a machine learning algorithm. FIG. 5F illustrates an example procedure 750 for determining a risk score for a user. The procedure 750 may be performed by the DHP 406, for example, the analytical subsystem 406a and/or the operational subsystem 406b. The procedure 750 may be performed periodically, for example, in response to alarm or schedule that may be configurable. For example, the procedure 750 may be performed on a periodic basis (e.g., performed every 15 minutes). Additionally or alternatively, the frequency at which the procedure 750 is performed may vary, for example, in response to amount of inhaler data received by the DHP 406 (e.g., when the amount of inhaler data received by the DHP 406 increases). As illustrated in FIG. 5F, the procedure 750 may enter at 751.

At 752, the DHP 406 may receive usage events associated with different users. The DHP 406 may receive the usage events from user devices (e.g., such as user devices 402a-d shown in FIGS. 4A and 4B) and/or inhalers 401a-d. Each usage event may be associated with a medicament type and a user of the different users. The usage events may include maintenance usage events associated with a maintenance medicament type and rescue usage events associated with a rescue medicament type. The maintenance usage events may be associated with a dosing schedule for the maintenance medicament type. Each usage event may include a time associated with the usage event and one or more inhalation parameters of the usage event. For example, the DHP 406 may retrieve raw records of specific instances of received inhaler data. A raw record of a specific instance of received inhaler data may include the raw information or data (e.g., in a certain format, such as JavaScript object notation (JSON) format) transmitted to the DHP 406 from a user device, as described herein. The raw information or data may include the time, inhalation parameters, etc.

At 754, the DHP 406 may identify the times and inhalation parameters associated with the usage events. After retrieving the raw inhaler data, the DHP 406 may parse the raw inhaler data to identify the times and inhalation parameters at 754. The inhalation parameters may include one or more of a PIF, an inhalation volume, a time to peak inhalation flow, an inhalation duration, etc.

At 756, the DHP 406 may determine a geographic location for the inhalation parameters. For example, the DHP 406 may receive location data associated with the inhalers (e.g., such as inhalers 401a-401d shown in FIG. 4A) and/or user devices (e.g., such as user devices 402a-402d shown in FIGS. 4A and 4B). The DHP 406 may tag each of the inhalation parameters with the determined geographic location. The DHP 406 may determine a point of interest based on the geographic location. The point of interest may include a park, a fuel station, a factory, a power plant, a highway, a user's home, a user's workplace, a train station, etc. The point of interest may be associated with the inhalation parameters. The point of interest(s) may be used to classify the inhalation parameters and/or train the machine learning algorithm. In examples, the geographic location may be identified using latitude and longitude coordinates, a geohash, a mapcode, or another type of geocoding system. The point of interest may comprise an area having a range of geographic locations (e.g., latitude and longitude coordinates, geohashes, mapcodes, etc.).

The DHP 406 may determine one or more environmental conditions for each usage event, for example, using the respective time and geographic location associated with each usage event. The environmental condition(s) may include one or more of temperature, humidity, outdoor air pollutant concentration, presence of particulate matter of 2.5 microns or smaller (PM2.5), presence of particulate matter of 10 microns or smaller (PM10), ozone concentration, nitrogen dioxide (NO₂) concentration, or sulfur dioxide (SO₂) concentration.

At 758, the DHP 406 may train a machine learning algorithm. The DHP 406 may train, at 406, the machine learning algorithm using training data. The training data may include compliance data, adherence data, and/or self-assessment response data. For example, the training data may include one or more of the times associated with the usage events, the inhalation parameters associated with the usage events, the geographic location, an adherence ratio, a number of rescue usage events in a predetermined number of days, a number of missed maintenance usage events in the predetermined number of days, a frequency of rescue usage events, a percent change in PIF, or a percent change in inhalation volume. The frequency of rescue usage events may be an average number of daily rescue usage events for the user over a predetermined period of time or an absolute number of daily rescue usage events for the user over the predetermined period of time. The predetermined period of time may be a predetermined number of days. The predetermined number of days may be a predetermined number of last days or days prior. The adherence ratio may indicate a user's adherence to a dosing schedule, for example, for a maintenance medicament type. For example, the adherence ratio may be determined based on a comparison between a number of maintenance usage events of the user over a predetermined period of time and a number of maintenance usage events indicated by the dosing schedule for the predetermined period of time. The machine learning algorithm may be trained, at 656, continuously (e.g., hourly, daily, weekly, etc.). For example, additional data received (e.g., after initial training) may be used to train the machine learning algorithm.

The machine learning algorithm may be trained, at 656, using an unsupervised learning method. An unsupervised learning method may be a type of machine learning that does not use labels for training data. The unsupervised learning method may be a learning method for learning artificial neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters.

At 760, the DHP 406 may determine a risk score for a user using the machine learning algorithm. The risk score may indicate a probability of an exacerbation of a respiratory condition for the user. The respiratory condition may include asthma, chronic obstructive pulmonary disease (COPD), and/or cystic fibrosis (CF). For example, the risk score may indicate a probability that the user will perform rescue usage event within a predetermined period of time. The predetermined period of time may comprise a predetermined (e.g., last predetermined) number of days. The risk score may be determined using pattern detection. For example, the machine learning algorithm may compare a pattern associated with the user to one or more patterns associated with a plurality of users. The pattern associated with the user may be determined using the user's training data over a previous number of days and the one or more patterns associated with the plurality of users may include the plurality of user's training data. The pattern may include one or more maintenance usage events, inhalation parameters associated with one or more maintenance usage events, one or more rescue usage events, inhalation parameters associated with one or more rescue usage events, and/or adherence to a dosing schedule over the predetermined period of time. The risk score may determined based on the geographic location and/or the environmental condition. For example, when an environmental condition changes, the risk score may be updated accordingly.

At 762, the DHP 406 may generate a notification indicating the risk score. The notification may be displayed for a practitioner and/or health care professional, for example, to allow them to analyze a user's exacerbation probability. In one example, the DHP 406 causes the computer 408 associated with the health care provider to provide the risk score and/or inhalation data via a graphical user interface (GUI) that is presented on the health care provider's computer. Additionally or alternatively, the notification may be displayed to the user via a display device (e.g., such as user devices 402a, 402b shown in FIG. 4A and/or user devices 402c, 402d shown in FIG. 4B). The notification indicating the risk score may provide feedback to the user of whether the user is likely to experience an exacerbation and/or need a rescue usage event in the predetermined period of time. For example, the notification indicating the risk score may be displayed for the health care professional and/or user based on a change to one or more environmental conditions that increase the risk score above a predetermined threshold.

Further, in some examples and as described herein, the DHP 406 may control an HVAC system associated with a user based on a score of the user (e.g., compliance score, future compliance score, and/or risk score). FIG. 5G illustrates an example procedure 800 for determining a score for a user. The procedure 800 may be performed by the DHP 406, for example, the analytical subsystem 406a and/or the operational subsystem 406b. The procedure 800 may be performed periodically, for example, in response to alarm or schedule that may be configurable. For example, the procedure 800 may be performed on a periodic basis (e.g., performed every 15 minutes). Additionally or alternatively, the frequency at which the procedure 800 is performed may vary, for example, in response to amount of inhaler data received by the DHP 406 (e.g., when the amount of inhaler data received by the DHP 406 increases). As illustrated in FIG. 5G, the procedure 800 may enter at 801.

At 802, the DHP 406 may receive usage events associated with different users. Each usage event may be associated with a medicament type and a user of the different users. The usage events may include maintenance usage events associated with a maintenance medicament type and rescue usage events associated with a rescue medicament type. The maintenance usage events may be associated with a dosing schedule for the maintenance medicament type. Each usage event may include a time associated with the usage event and one or more inhalation parameters of the usage event. For example, the DHP 406 may retrieve raw records of specific instances of received inhaler data. A raw record of a specific instance of received inhaler data may include the raw information or data (e.g., in a certain format, such as JavaScript object notation (JSON) format) transmitted to the DHP 406 from a user device, as described herein. The raw information or data may include the time, inhalation parameters, etc.

At 804, the DHP 406 may identify the times and inhalation parameters associated with the usage events. After retrieving the raw inhaler data, the DHP 406 may parse the raw inhaler data to identify the times and inhalation parameters at 804. The inhalation parameters may include one or more of a PIF, an inhalation volume, a time to peak inhalation flow, an inhalation duration, etc.

At 806, the DHP 406 may determine a geographic location for the inhalation parameters. For example, the DHP 406 may receive location data associated with the inhalers (e.g., such as inhalers 401a-401d shown in FIG. 4A) and/or user devices (e.g., such as user devices 402a-402d shown in FIGS. 4A and 4B). The DHP 406 may tag each of the inhalation parameters with the determined geographic location. The DHP 406 may determine a point of interest based on the geographic location. The point of interest may include a park, a fuel station, a factory, a power plant, a highway, a user's home, a user's workplace, a train station, etc. The point of interest may be associated with the inhalation parameters. The point of interest(s) may be used to classify the inhalation parameters and/or train the machine learning algorithm. In examples, the geographic location may be identified using latitude and longitude coordinates, a geohash, a mapcode, or another type of geocoding system. The point of interest may comprise an area having a range of geographic locations (e.g., latitude and longitude coordinates, geohashes, mapcodes, etc.).

At 808, the DHP 406 may determine one or more environmental conditions associated with the geographic location. For example, the environmental condition(s) may be determined, at 808, for each usage event using the respective time and geographic location associated with the respective usage event. The environmental condition(s) may include one or more of temperature, humidity, outdoor air pollutant concentration, presence of particulate matter of 2.5 microns or smaller (PM2.5), presence of particulate matter of 10 microns or smaller (PM10), ozone concentration, nitrogen dioxide (NO₂) concentration, or sulfur dioxide (SO₂) concentration.

At 810, the DHP 406 may train a machine learning algorithm. The DHP 406 may train, at 810, the machine learning algorithm using training data. The training data may include compliance data, adherence data, and/or self-assessment response data. For example, the training data may include one or more of the times associated with the usage events, the inhalation parameters associated with the usage events, the geographic location, the environmental condition(s), an adherence ratio, a number of rescue usage events in a predetermined number of days, a number of missed maintenance usage events in the predetermined number of days, a frequency of rescue usage events, a percent change in PIF, or a percent change in inhalation volume. The frequency of rescue usage events may be an average number of daily rescue usage events for the user over a predetermined period of time or an absolute number of daily rescue usage events for the user over the predetermined period of time. The predetermined period of time may be a predetermined number of days. The predetermined number of days may be a predetermined number of last days or days prior. The adherence ratio may indicate a user's adherence to a dosing schedule, for example, for a maintenance medicament type. For example, the adherence ratio may be determined based on a comparison between a number of maintenance usage events of the user over a predetermined period of time and a number of maintenance usage events indicated by the dosing schedule for the predetermined period of time. The machine learning algorithm may be trained, at 810, continuously (e.g., hourly, daily, weekly, etc.). For example, additional data received (e.g., after initial training) may be used to train the machine learning algorithm.

The machine learning algorithm may be trained, at 810, using an unsupervised learning method. An unsupervised learning method may be a type of machine learning that does not use labels for training data. The unsupervised learning method may be a learning method for learning artificial neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters.

At 812, the DHP 406 may determine a score for a user using the machine learning algorithm, for example, using one or more of the procedures described herein, such as the procedure 600 (e.g., to determine an individualized compliance score), 650 (e.g., to determine an individualized future compliance score), and/or 750 (e.g., to determine an individualized risk score).

At 814, the DHP 406 may control an HVAC system associated with the user based on the score for the user. The DHP 406 may control, at 814, the HVAC system to adjust a humidity level, temperature, etc. of the user's home, user's workplace, etc. For example, the DHP 406 may be configured to adjust the humidity level inside the user's home below a humidity threshold associated with the user's score. Additionally or alternatively, the DHP 406 may be configured to adjust the temperature inside the user's home above or below a temperature threshold associated with the user's score. The proximity of the user to the point of interest may be used as a trigger to adjust the HVAC system. For example, the DHP 406 may be configured to determine whether the user is within a predetermined distance from a point of interest. When the DHP 406 determines that the user is within the predetermined distance from the point of interest, the DHP 406 may control the HVAC to adjust one or more parameters based on the score for the user.

As noted above, the DHP 406 may communicate with a computer or server 408 that comprises (e.g., runs or otherwise interacts with) a HCP facing processing module (e.g., also referred to herein as a Dashboard application) associated with a health care provider, such as a hospital or hospital system, a health system, a medical group, a physician, a clinic, and/or a pharmaceutical company.

The DHP 406 may cause the computer 408 associated with the health care provider to provide inhaler data, compliance scores, future compliance scores, and/or risk scores to practitioners and health care professionals to allow them to view inhaler data specific to a program to which a patient has consented. In one example, the DHP 406 causes the computer 408 associated with the health care provider to provide the inhalation data, compliance scores, future compliance scores, and/or risk scores via a graphical user interface (GUI) that is presented on the health care provider's computer. Each GUI may be, for example, specific for a particular program (as described above), and may provide any combination of the name of the users (e.g., patients), the date-of-birth of the users, and inhaler data that is divided in columns for each specific medicament type that is part of the program. The GUI may provide specific information for each user, and for example, the information may be aggregated by medicament type for all of the user's inhalers 401 that provide a particular medicament type. For instance, the GUI may provide a total number of usage events (e.g., inhalation events, such as, good inhalation events, fair inhalation events, low or no inhalation events, exhalation events, and possible air vent block events) that have occurred over a period of time, such as one week or 40 days, for all inhalers 401 that include a particular medicament type, a percentage of the inhalation events that are categorized as good inhalation events, and an all-connected duration that represents the oldest, or least recent, last synchronization time of the inhalers 401 of a particular medication type that are associated with the user.

HCPs have a limited amount of time and resources to analyze data associated with their patient's use of their medical devices. And further, different usage characteristics, such as underuse, overuse, and various types of improper use of a medical device, may have varying degrees of relevance or importance to a physician based on the type of medical device and/or the medicament type provided by the medical device. For example, for some medicament types, an indication of overuse is more concerning than an indication over underuse, or vice versa. For instance, overuse of a rescue medicament inhaler suggests that the user is experiencing a high number of exacerbations and has poor control of their respiratory condition. And an underuse of a maintenance inhaler indicates that the user is not adhering to their prescribed dosing schedule, which suggests that the user has not been properly informed as to their dosing regimen, that the user is overprescribed and does not need that particular dose concentration or regimen and is cutting back intentionally, and/or that the user is not taking their respiratory condition seriously.

Further, in some examples, the DHP 406 is configured to prioritize the listing of users on the GUI based on the particular medicaments that are included in the program. For instance, as noted above, the DHP 406 may be configured to prioritize the listing of users on the GUI to first indicate those users with the highest number of usage events (e.g., over a particular time range, such as one week) of inhalers 401 that include a rescue medicament type, when the rescue medicament type and the maintenance medicament type are included in the program. In such examples, the DHP 406 is also configured to prioritize the listing of users on the GUI to first indicate those users with the lowest number of usage events (e.g., over a particular time range, such as one month) of inhalers 401 that include the maintenance medicament type when the rescue medicament type is not included in the program.

Further, in some examples, the DHP 406 may be configured to prioritize the listing of users on the GUI based on the compliance score, the future compliance score, and/or the risk score. For instance, as noted above, the DHP 406 may be configured to prioritize the listing of users on the GUI to first indicate those users with the lowest compliance scores, the lowest future compliance scores, and/or the highest risk scores.

The DHP 406 may cause the computer or server 408 associated with (e.g., operated by) the health care provider to provide inhaler data to practitioners and health care professionals to allow them to view inhaler data specific to a program to which a patient has consented, for example, via a Care Provider facing processing module at the computer or server 408. In one example, the DHP 406 causes the computer or server 408 associated with the health care provider to provide the inhalation data via a GUI that is presented on a display device associated with the health care provider's computer.

The DHP 406 may define any number of programs, which in some instances may be configured and altered by a health care provider. When providing inhaler data to a health care provider, the DHP 406 may provide a GUI (e.g., may be configured to display a GUI) that is specific to a particular program associated with that health care provider. A program defines a set of criteria, such as types of medications (e.g., any combination of rescue and/or maintenance medications), specific patients and in turn their applicable inhalers, other users of the programs such as particular physicians, practice groups, and/or administrators, the types of data presented to the health care provider such as charts, event tables, usage summaries, etc. The health care provider may configure and establish any number of programs using the DHP 406. Further, a particular patient and their inhalers may be associated with any number of unique programs. In some examples, the programs are stored and maintained by the DHP 406, and the computer associated with the health care provider is configured to access the data relevant to each program from the DHP 406 using, for example, an application, such as a dashboard or web application. In such examples, and once a program is established, the DHP 406 is configured to receive inhaler data associated with the program, analyze and manipulate the inhaler data to the extent necessary, and provide program data (e.g., via the dashboard) to the health care provider. The program data may include inhaler data that is specific to the configuration of a particular program, compliance scores, future compliance scores, risk scores, and/or for example, additional data that is derived from the inhaler data, as is described in more detail below. For example, the DHP 406 may enable a GUI, such as those described herein, on the computer associated with the health care provider that presents the program data to the health care provider.

The DHP 406 is configured to manipulate data differently and/or generate different notifications/alerts based on the specific medications, patients, users (e.g., health care provides), and/or therapeutic devices or areas defined by the program. As one example, the DHP 406 may be configured to generate a first type of alert when a rescue medicament type and one or more maintenance medicament types are included in the program, but generate a second type of alert when the rescue medicament type is not included in the program. For instance, the DHP 406 may be configured to generate a first alert that indicates a prioritized listing of users with the highest number of usage events (e.g., over a particular time range, such as one week) of inhalers that include a rescue medicament type when the rescue medicament type and one or more maintenance medicament types are included in the program, and generate a second alert that indicates a prioritized listing of users with the lowest number of usage events (e.g., over a particular time range, such as one month) of inhalers that include the maintenance medicament type when the rescue medicament type is not included in the program. In some examples, the alert is provided via a display device that, for example, may reside at a health care provider facility.

For example, the DHP 406 may be configured to prioritize data, such as patient lists, differently based on the specific medication types, patients, users (e.g., health care providers), and/or therapeutic devices or areas defined by the program. In some examples, the DHP 406 is configured to prioritize data indicating high usage of a rescue inhaler over data indicating a low usage of a rescue inhaler and over data indicating either a high or low usage of a maintenance inhaler. For example, if a program includes rescue medicaments and one or more maintenance medicaments for a group of users associated with the program, the DHP 406 may be configured to prioritize the users with the highest number of usage events of their inhaler(s) that include the rescue medicament over the users with a lower number of usage events of their inhaler(s) that include the rescue medicament and over the users with the highest or lowest number of usage events of their inhaler(s) with a maintenance medicament. However, if rescue inhalers are not part of the program, then the DHP 406 is configured to prioritize users with the lowest number of usage events of their inhalers(s) that include a particular maintenance medicament, over users with the highest number of usage events of their inhaler(s) that include the particular maintenance medicament. Further, in some instances, the DHP 406 may prioritize users with the highest usage events of their inhaler(s) that include the rescue medicament only when there is at least one user in the program who has a number of usage events for their rescue inhaler(s) that exceeds a threshold amount in a period of time, such as 5 usage events within the past week, and otherwise, may prioritize the users with the lowest number of usage events of their inhalers(s) that include a particular maintenance medicament.

The DHP 406 may be configured to determine an all-connected duration for each medicament type associated with each user. For example, the DHP 406 may be configured to determine a group of inhalers that are all associated with one user and that all include the same medicament. The DHP 406 may be configured to then determine the last synchronization time for all of those inhalers. Next, the DHP 406 may be configured to determine the oldest or least recent last synchronization time from the group and set this time as the an all-connected duration for that particular medicament type for that particular user. The DHP 406 may be configured to generate an alert that indicates the all-connected duration, the medicament type, and the user. In some examples, the alert is provided via a display device that, for example, may reside at a health care provider facility.

For example, in a non-limiting example, the DHP 406 is configured to prioritize users based on the all-connected time for a particular medicament type. For instance, the DHP 406 may generate an alert (e.g., a GUI) that prioritizes users with the oldest all-connected time for their inhaler(s) of a particular maintenance medicament type, when that maintenance medicament is part of the program, but otherwise, prioritize users in another manner, such as based on the users with the highest number of usage events of their inhaler(s) that include the rescue medicament or based on the users with the lowest percentage of good inhalations (e.g., while excluding all users with 0% good inhalations), possible of a particular medicament type. As such, the DHP 406 may be configured to prioritize data, such as patient lists, differently based on the specific medication types that are included in the program.

As noted above, the DHP 406 is configured to receive and aggregate inhaler data from inhalers that are associated with a plurality of different users. However, particularly in situations where the inhaler is configured to first communicate with a user device prior to the DHP 406 receiving the inhaler data, the DHP 406 may not have a complete history of usage events for each inhaler that is part of a program. For example, there may be some inhalers that have recorded usage events but have not connected to an associated user device, and as such, have not transferred their usage events to the user device or to the DHP 406. But, the DHP 406 may calculate zero usage events for two different inhalers, when in fact, one of those inhalers may have recorded usage events but has not transferred those usage events to the DHP 406, while the other inhaler has in-fact not been used. That is, the DHP 406 may include data from many inhalers that have zero dosage events over a particular period of time, such as the past week or month. However, some of these inhalers may not have actually been used by their user, while other inhalers may or may not have been used, but the DHP 406 may be unaware of the reliability of the usage data. This can distort the data presented by the DHP 406 and cause confusion for health care providers who are reviewing the data relating to one of their programs. For example, listing inhalers (or users associated with inhalers) based on the least number of usage events will create a distorted view of inhalers by comingling inhalers that have not had any usage events with inhalers that have had one or more usage events, but the data associated with these events has not yet been received by the DHP 406. Therefore, sorting on a descending basis of the number of usage events will result in a list of inhalers that does not provide an accurate indication of those users who are not adherent with their dosing regimen.

As such, the DHP 406 may be configured to determine and differentiate users who are associated with inhalers that have a true zero number of usage events from users who are associated with inhalers that appear to have zero usage events from the DHP's 406 perspective but have not connected to their associated user device (or directly to the DHP 406) within a particular time period. For example, the DHP 406 may be configured to determine a last synchronization time for each of the plurality of inhalers. The last synchronization time indicates the last time the inhaler was connected with a user device that includes a mobile application (e.g., or, alternatively, connected directly to the DHP 406), regardless of whether usage events were transferred as part of the connection. Further, it should be appreciated that some inhalers may be configured to be connected to multiple different user devices (e.g., such as a user's smartphone and their tablet), and as such, the last synchronization time indicates the last the inhaler was connected with any user device that includes a mobile application.

The DHP 406 may calculate the last synchronization time using the time stamps that are associated with usage events. For example, when the inhaler connects with the processing module residing on the user device, the processing module may indicate a time denoting the last time the inhaler connected to a mobile application or a time of the most recent usage event acquired from the inhaler. In response, the inhaler may send only those usage events that postdate that time. The processing module may later send these usage events and an indication of the time of the connection between the inhaler and the processing module to the DHP 406. For example, the processing module on the user device may be configured to send inhalation data (or an indication that there is no new inhalation data) to the DHP 406 periodically, such as every 15 minutes. As such, the DHP 406 may determine a last synchronization time for each inhaler that indicates the last time the inhaler was connected with a user device that includes a mobile application (e.g., or, alternatively, connected directly to the DHP 406), for example, regardless of whether usage events were transferred as part of the connection.

The DHP 406 may be configured to generate an alert that indicates the users that are associated with an inhaler with zero doses and a confirmed connection within a time frame. In some examples, the DHP 406 may cause a display device (e.g., associated with a health care provider) to present an alert (e.g., a GUI) that includes a listing of users, where the listing prioritizes users with an inhaler that has zero usage events and a confirmed connection between the inhaler and the application residing on the user device within a time frame over users with an inhaler that has zero usage events without a confirmed connection between the inhaler and the application residing on the user device within the time frame. In some examples, the inhalers all include a maintenance medicament type. For example, the program may be limited to inhalers that include a maintenance medicament type. Further, in some examples, the time frame may be the last month or the last 30 days.

As noted above, some users will have multiple inhalers that include the same medicament. For example, a user may have multiple inhalers that include a rescue medicament (e.g., and keep them at different locations). Further, a user may have multiple inhalers that include a particular maintenance medicament, such as when they are transitioning between refills. As such, the DHP 406 may be unaware of how complete and trustworthy the data is for each patient in instances where a patient has multiple inhalers that include the same medicament, because for example, there still may be instances where an inhaler has not connected with the user device for a period of days, weeks, or even months. Also as noted above, the DHP 406 is configured to determine a last synchronization time for each of the plurality of inhalers. Further, the DHP 406 may also be configured to determine whether the user has multiple assigned inhalers that include the same medicament (e.g., such as a rescue medicament), and then determine the oldest, or least recent, last synchronization time of the inhalers of a particular medication type that are associated with the user. This may be referred to as an all-connected duration, which represents the oldest, or least recent, last synchronization time of all the inhalers of a particular medication type that are associated with the user. For example, if a user is associated with two inhalers that include a rescue medicament, such as inhaler 1 and inhaler 2, and inhaler 1 has a last synchronization time of 2 days ago, but inhaler 2 has a last synchronization time of 10 days ago, the user will be associated with an all-connected duration for their rescue medicament inhalers of 10 days ago.

The DHP 406 may be configured to generate an alert (e.g., a GUI) that indicates a listing of a plurality of users and the all-connected duration for each medicament type associated with each user. As noted above, the all-connected duration may indicate the last time that all of the inhalers of a particular medication type associated with the user have a confirmed connection between the inhaler and the application residing on the user device associated with the user, for example, regardless of whether a usage event was transferred during the connection. In some examples, the all-connected duration indicates a number of hours, days, weeks, or even months.

The system may include devices and users that are located in various different time zones, which may fluctuate from day-to-day. As such, the data captured by the system may also be associated with various time zones. However, the inhalers may be unaware of their time zone (e.g., they may only include short range personal area network communication capability), and the health care provider may be unaware of the time zone of usage events for each user. Nonetheless, the DHP 406 is challenged when determining a cohesive manner to provide an alert or feedback to a health care provider when the data is associated with a variety of different time zones, medicament types, and usage events.

As noted above, in some examples, the use determination system of an inhaler may start an internal counter (e.g., which counts up from 0 indefinitely) when, for example, the use determination system is woken out of an energy-saving sleep mode for the first time (e.g., after the mouthpiece cover is opened for the first time). Thereafter, any time-and-date stamp generated by the use determination system may be a relative time (or count) based on the internal counter of the clock module. And, when the inhaler connects with the processing module residing on the user device, the processing module may indicate a time denoting the last time the inhaler connected to a mobile application or a time of the most recent usage event acquired from the inhaler. In response, the inhaler may send only those usage events that postdate that time. The processing module may receive one or more usage events and associated timestamps (e.g., a relative count from the internal counter) from the inhaler. The processing module may determine a local mean time and a time zone for a timestamp, and associated the local mean time and time zone with the usage event. The time zone may be that of the processing module at the time of receiving the usage event, or may be the time zone of the processing module at the time associated with the usage event. The processing module may then send the usage event and the associated local mean time and time zone to the DHP 406. The DHP 406 may associate the usage event, local mean time, and time zone with a user.

Next, when accessed by a health care professional, the DHP 406 may generate notifications/alerts that are specific to the users, inhalers, and/or medicaments of the program selected by the health care professional based on the time zones of the most recent usage event associated with the user. For example, the DHP 406 may determine a time frame (e.g., time window) based on the medicament type and the most recent usage event. And the DHP 406 may generate an alert (e.g., GUI) that indicates all the user's usage events within the time window. The DHP 406 may determine the time window based on the most recent usage event, based on the time zone of the usage event, plus an addition N days (e.g., where N=6 for rescue medicament, and N=29 or 30 for maintenance medicament). For instance, if the medicament type is a rescue medicament type, and the user's most recent usage event occurred at 8:30 am Jun. 30, 2020 (GMT+9), then the time window will include Jun. 30, 2020 and the previous 6 days. However, if the medicament type is a rescue medicament type, and the user's most recent usage event occurred at 7:30 pm on Jun. 29, 2020 (GMT-5), then the time window will include Jun. 29, 2020 and the previous 6 days. Therefore, although the usage event occurred at the same time, the time window for each alert is selected differently based on the local time zone of the most recent usage event for that user (e.g., regardless of the time zone of the health care provider).

Further, when accessed by a health care professional, the DHP 406 may determine a time zone of the health care professional, and the DHP 406 may generate notifications/alerts that are specific to the users, inhalers, and/or medicaments of the program selected by the health care professional based on the time zone of the health care professional (e.g., in addition to, or in lieu of the notifications/alerts that are based on the time zones of the most recent usage event associated with the user). For example, the DHP 406 may generate notifications/alerts that indicate a plurality of usage events, the user associated with each usage event, and the associated local mean time of the usage event. Then, only for the usage events that are associated with a time zone that is different from the time zone of the health care professional, the DHP 406 may also generate notifications/alerts that indicate the time zone of the usage event. So, in some examples, the DHP 406 may determine a time frame (e.g., time window) based on the medicament type and the most recent usage event for a user, and then generate an alert (e.g., GUI) that indicates all the user's usage events within the time window for that user, while also including the time zone for each usage event only where it differs from the time zone associated with the health care provider.

Further, although described primarily with respect to inhaler data, it should be appreciated that the system described herein may aggregate data from any combination of therapeutic devices or areas. In such examples, the DHP 406 may configure programs that also define particular therapeutic devices or areas, such as those described herein. That is, the DHP 406 is configured to maintain and analyze data for a plurality of disease types, medical devices, and/or medications.

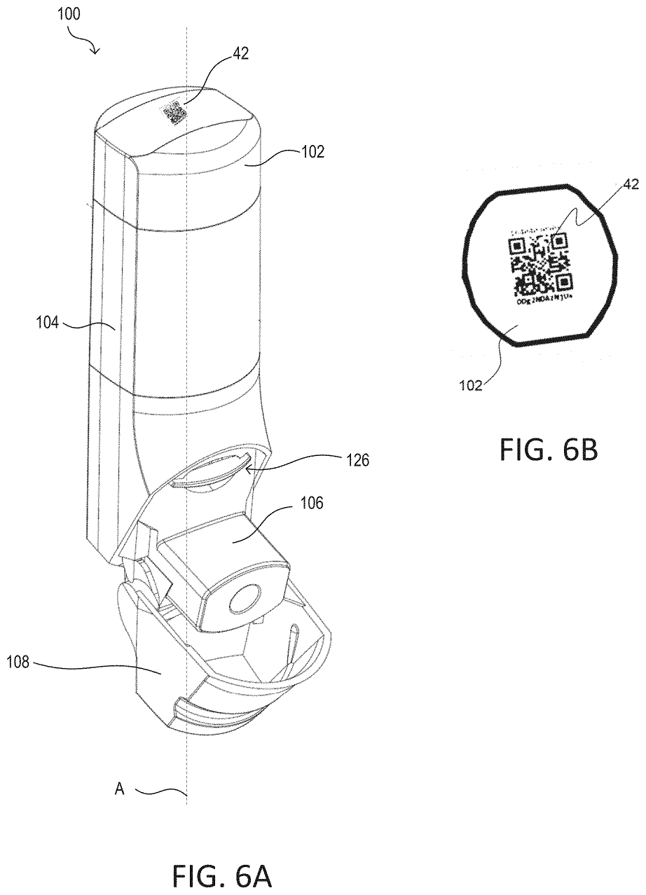

FIGS. 6A, 6B, and 7-9 provide a non-limiting example arrangement of the inhalers 100, 100A-C, and 401a-d that may be included in the system 400 illustrated in FIGS. 4A and 4B. FIG. 6A provides a front perspective view of an inhaler arrangement 100, referred to as “an inhaler” from here on, according to a non-limiting example. The inhaler 100 may, for example, be a breath-actuated inhaler. The inhaler 100 may include a top cap 102, a main housing 104, a mouthpiece 106, a mouthpiece cover 108, an electronics module 120, and an air vent 126. The mouthpiece cover 108 may be hinged to the main housing 104 so that it may open and close to expose the mouthpiece 106. Although illustrated as a hinged connection, the mouthpiece cover 106 may be connected to the inhaler 100 through other types of connections. Moreover, while the electronics module 120 is illustrated as housed within the top cap 102 at the top of the main housing 104, the electronics module 120 may be integrated and/or housed within the main body 104 of the inhaler 100.

The electronics module 120 may, for instance, include the above-described use determination system 12 and the transmission module 14. For example, the electronics module 120 may include a processor, memory configured to perform the functions of use determination system 12 and/or transmission module 14. The electronics module 120 may include switch(es), sensor(s), slider(s), and/or other instruments or measurement devices configured to determine inhaler usage information as described herein. The electronics module 120 may include a transceiver and/or other communication chips or circuits configured to perform the transmission functions of transmission module 14.

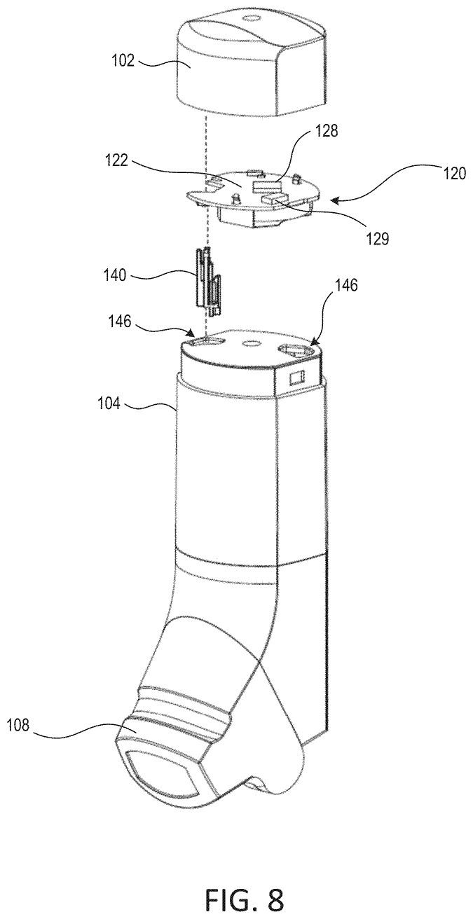

In the non-limiting example shown in FIG. 6A, the inhaler 100 has a barcode 42 printed thereon. The barcode 42 in this example is a quick reference (QR) code printed on the uppermost surface of the top cap 102. The use determination system 12 and/or the transmission module 14 may, for example, be located at least partly within the top cap 102, for example as components of an electronics module (not visible in FIG. 6A). The electronics module of the inhaler 100 will be described in greater detail with reference to FIGS. 8 to 10.

The QR code is more clearly visible in FIG. 6B which provides a view from directly above the top cap 102 of the inhaler 100 shown in FIG. 6A. The QR code 42 may provide a facile way of pairing the respective inhaler 100 with the processing module 34, in examples in which the user device 40 comprises a suitable optical reader, such as a camera, for reading the QR code.

Such a bar code 42, e.g. QR code, may comprise the identifier which is assigned to the respective medicament of the inhaler 100, as previously described. Table 2 provides a non-limiting example of the identifiers included in the QR code 42 for various inhalers 100.

TABLE 2

| | | | Dose | | Medicament |
|---|---|---|---|---|---|
| Identifier | Brand of | | strength | Total dose count of | identification |
| in QR code | inhaler | Medicament | (mcg) | inhaler prior to use | number |
| <blank> | ProAir | albuterol | 117 | 200 | AAA200 |
| | Digihaler | | | | |
| AAA030 | ProAir | albuterol | 117 | 30 | AAA030 |
| | Digihaler | | | | |
| FSL060 | AirDuo | fluticasone/ | 55/14 | 60 | FSL060 |
| | Digihaler | salmeterol | | | |
| FSM060 | AirDuo | fluticasone/ | 113/14 | 60 | FSM060 |
| | Digihaler | salmeterol | | | |
| FSH060 | AirDuo | fluticasone/ | 232/14 | 60 | FSH060 |
| | Digihaler | salmeterol | | | |
| FPL060 | ArmonAir | fluticasone | 55 | 60 | FPL060 |
| | Digihaler | | | | |
| FPM060 | ArmonAir | fluticasone | 113 | 60 | FPM060 |
| | Digihaler | | | | |
| FPH060 | ArmonAir | fluticasone | 232 | 60 | FPH060 |
| | Digihaler | | | | |

As shown in Table 2, the identifier further denotes the dose strength and the total dose count of the inhaler prior to use. The processing module 34 may use the former to, in combination with the usage information, control the user interface 38 to issue a notification when the label recommended dosages have been exceeded, as previously described. Alternatively or additionally, the processing module 34 may use the total dose count of the inhaler prior to use and the usage information to determine the number of doses remaining in the respective inhaler 100, as previously described.

The barcode 42, e.g. QR code, on the inhaler may, for instance, further comprise a security key, for example in the form of a series of alphanumerical characters, for preventing unauthorized users from accessing the respective inhaler 100. The processing module 34 may be able to decrypt the respective encrypted data once the processing module 34 has been provided with the security key, but may not be able to decrypt the respective encrypted data before the processing module 34 has been provided with the security key. More generally, the security key may be included in the respective identifier.

In a non-limiting example, the system is configured to restrict one or more, e.g. each, of the inhalers included in the system to a single user account.

In such an example, a passkey, e.g. provided in the QR code, may allow synchronization between the respective inhaler and the processing module of the system. The passkey and, in turn, the usage parameter data, e.g. inhalation and/or usage data, from the respective inhaler may be public. This public inhalation data may not be associated with the particular subject until synchronization with the single user account.

Since the system is configured to restrict the respective inhaler to being associated with the single user account, the respective inhaler may be prevented from being synchronized with another user account, for example in situations where the inhaler is lost or stolen. In this way, third parties may be prevented from acquiring usage parameter data which is not theirs.

In other non-limiting examples, the processing module 34 may be paired with the respective inhaler 100 by, for example, manual entry of an alphanumerical key including the respective identifier via the user interface, e.g. a touchscreen.

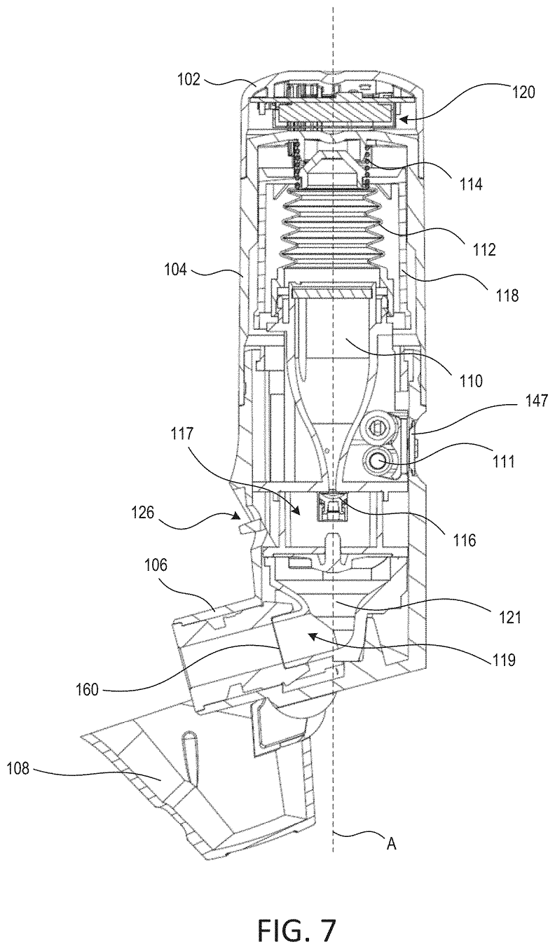

FIG. 7 provides a cross-sectional interior perspective view of the example inhaler 100. Inside the main housing 104, the inhaler 100 may include a medication reservoir 110 and a dose metering assembly. For example, the inhaler 100 may include a medication reservoir 110 (e.g. a hopper), a bellows 112, a bellows spring 114, a yoke (not visible), a dosing cup 116, a dosing chamber 117, a deagglomerator 121, and a flow pathway 119. The medication reservoir 110 may include medication, such as dry powder medication, for delivery to the subject. Although illustrated as a combination of the bellows 112, the bellows spring 114, the yoke, the dosing cup 116, the dosing chamber 117, and the deagglomerator 121, the dose metering assembly may include a subset of the components described and/or the inhaler 100 may include a different dose metering assembly (e.g., based on the type of inhaler, the type of medication, etc.). For instance, in some examples the medication may be included in a blister strip and the dose metering assembly, which may include one or more wheels, levers, and/or actuators, is configured to advance the blister strip, open a new blister that includes a dose of medication, and make that dose of medication available to a dosing chamber and/or mouthpiece for inhalation by the user.

When the mouthpiece cover 108 is moved from the closed to the open position, the dose metering assembly of the inhaler 100 may prime a dose of medicament. In the illustrated example of FIG. 7, the mouthpiece cover 108 being moved from the closed to the open position may cause the bellows 112 to compress to deliver a dose of medication from the medication reservoir 110 to the dosing cup 116. Thereafter, a subject may inhale through the mouthpiece 106 in an effort to receive the dose of medication.

The airflow generated from the subject's inhalation may cause the deagglomerator 121 to aerosolize the dose of medication by breaking down the agglomerates of the medicament in the dose cup 116. The deagglomerator 121 may be configured to aerosolize the medication when the airflow through the flow pathway 119 meets or exceeds a particular rate, or is within a specific range. When aerosolized, the dose of medication may travel from the dosing cup 116, into the dosing chamber 117, through the flow pathway 119, and out of the mouthpiece 106 to the subject. If the airflow through the flow pathway 119 does not meet or exceed a particular rate, or is not within a specific range, the medication may remain in the dosing cup 116. In the event that the medication in the dosing cup 116 has not been aerosolized by the deagglomerator 121, another dose of medication may not be delivered from the medication reservoir 110 when the mouthpiece cover 108 is subsequently opened. Thus, a single dose of medication may remain in the dosing cup until the dose has been aerosolized by the deagglomerator 121. When a dose of medication is delivered, a dose confirmation may be stored in memory at the inhaler 100 as dose confirmation information.

As the subject inhales through the mouthpiece 106, air may enter the air vent to provide a flow of air for delivery of the medication to the subject. The flow pathway 119 may extend from the dosing chamber 117 to the end of the mouthpiece 106, and include the dosing chamber 117 and the internal portions of the mouthpiece 106. The dosing cup 116 may reside within or adjacent to the dosing chamber 117. Further, the inhaler 100 may include a dose counter 111 that is configured to be initially set to a number of total doses of medication within the medication reservoir 110 and to decrease by one each time the mouthpiece cover 108 is moved from the closed position to the open position.

The top cap 102 may be attached to the main housing 104. For example, the top cap 102 may be attached to the main housing 104 through the use of one or more clips that engage recesses on the main housing 104. The top cap 102 may overlap a portion of the main housing 104 when connected, for example, such that a substantially pneumatic seal exists between the top cap 102 and the main housing 104.

FIG. 8 is an exploded perspective view of the example inhaler 100 with the top cap 102 removed to expose the electronics module 120. As shown in FIG. 8, the top surface of the main housing 104 may include one or more (e.g. two) orifices 146. One of the orifices 146 may be configured to accept a slider 140. For example, when the top cap 102 is attached to the main housing 104, the slider 140 may protrude through the top surface of the main housing 104 via one of the orifices 146.

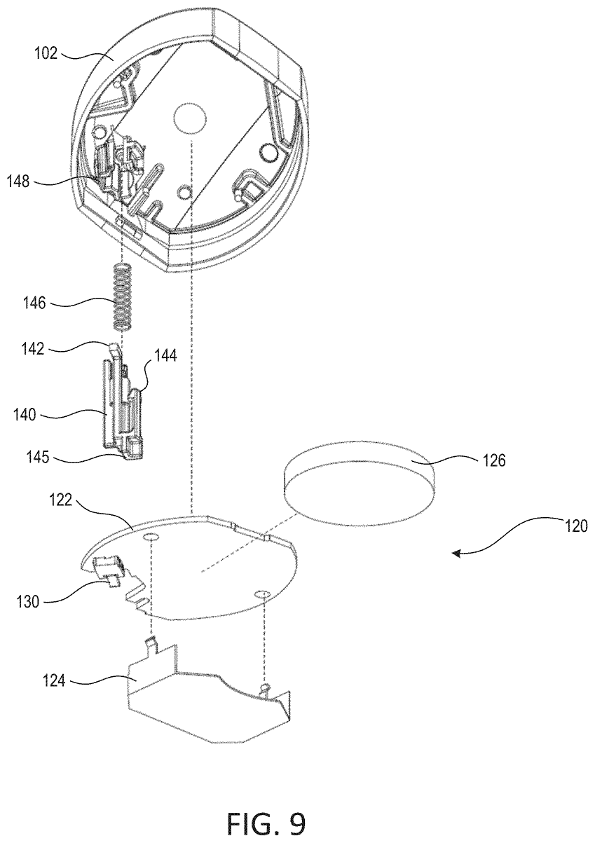

FIG. 9 is an exploded perspective view of the top cap 102 and the electronics module 120 of the example inhaler 100. As shown in FIG. 9, the slider 140 may define an arm 142, a stopper 144, and a distal end 145. The distal end 145 may be a bottom portion of the slider 140. The distal end 145 of the slider 140 may be configured to abut the yoke that resides within the main housing 104 (e.g. when the mouthpiece cover 108 is in the closed or partially open position). The distal end 145 may be configured to abut a top surface of the yoke when the yoke is in any radial orientation. For example, the top surface of the yoke may include a plurality of apertures (not shown), and the distal end 145 of the slider 140 may be configured to abut the top surface of the yoke, for example, whether or not one of the apertures is in alignment with the slider 140.

The top cap 102 may include a slider guide 148 that is configured to receive a slider spring 146 and the slider 140. The slider spring 146 may reside within the slider guide 148. The slider spring 146 may engage an inner surface of the top cap 102, and the slider spring 146 may engage (e.g. abut) an upper portion (e.g. a proximate end) of the slider 140. When the slider 140 is installed within the slider guide 148, the slider spring 146 may be partially compressed between the top of the slider 140 and the inner surface of the top cap 102. For example, the slider spring 146 may be configured such that the distal end 145 of the slider 140 remains in contact with the yoke when the mouthpiece cover 108 is closed. The distal end 145 of the slider 145 may also remain in contact with the yoke while the mouthpiece cover 108 is being opened or closed. The stopper 144 of the slider 140 may engage a stopper of the slider guide 148, for example, such that the slider 140 is retained within the slider guide 148 through the opening and closing of the mouthpiece cover 108, and vice versa. The stopper 144 and the slider guide 148 may be configured to limit the vertical (e.g. axial) travel of the slider 140. This limit may be less than the vertical travel of the yoke. Thus, as the mouthpiece cover 108 is moved to a fully open position, the yoke may continue to move in a vertical direction towards the mouthpiece 106 but the stopper 144 may stop the vertical travel of the slider 140 such that the distal end 145 of the slider 140 may no longer be in contact with the yoke.

More generally, the yoke may be mechanically connected to the mouthpiece cover 108 and configured to move to compress the bellows spring 114 as the mouthpiece cover 108 is opened from the closed position and then release the compressed bellows spring 114 when the mouthpiece cover reaches the fully open position, thereby causing the bellows 112 to deliver the dose from the medication reservoir 110 to the dosing cup 116. The yoke may be in contact with the slider 140 when the mouthpiece cover 108 is in the closed position. The slider 140 may be arranged to be moved by the yoke as the mouthpiece cover 108 is opened from the closed position and separated from the yoke when the mouthpiece cover 108 reaches the fully open position. This arrangement may be regarded as a non-limiting example of the previously described dose metering assembly, since opening the mouthpiece cover 108 causes the metering of the dose of the medicament.

The movement of the slider 140 during the dose metering may cause the slider 140 to engage and actuate a switch 130. The switch 130 may trigger the electronics module 120 to register the dose metering. The slider 140 and switch 130 together with the electronics module 120 may thus be regarded as being included in the use determination system 12 described above. The slider 140 may be regarded in this example as the means by which the use determination system 12 is configured to register the metering of the dose by the dose metering assembly, each metering being thereby indicative of the inhalation performed by the subject using the inhaler 100.

Actuation of the switch 130 by the slider 140 may also, for example, cause the electronics module 120 to transition from the first power state to a second power state, and to sense an inhalation by the subject from the mouthpiece 106.

The electronics module 120 may include a printed circuit board (PCB) assembly 122, a switch 130, a power supply (e.g. a battery 126), and/or a battery holder 124. The PCB assembly 122 may include surface mounted components, such as a sensor system 128, a wireless communication circuit 129, the switch 130, and or one or more indicators (not shown), such as one or more light emitting diodes (LEDs). The electronics module 120 may include a controller (e.g. a processor) and/or memory. The controller and/or memory may be physically distinct components of the PCB 122. Alternatively, the controller and memory may be part of another chipset mounted on the PCB 122, for example, the wireless communication circuit 129 may include the controller and/or memory for the electronics module 120. The controller of the electronics module 120 may include a microcontroller, a programmable logic device (PLD), a microprocessor, an application specific integrated circuit (ASIC), a field programmable gate array (FPGA), or any suitable processing device or control circuit.

The controller may access information from, and store data in the memory. The memory may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory may be internal to the controller. The controller may also access data from, and store data in, memory that is not physically located within the electronics module 120, such as on a server or a smart phone.

The sensor system 128 may include one or more sensors. The sensor system 128 may be, for example, included in the use determination system 12 described above. The sensor system 128 may include one or more sensors, for example, of different types, such as, but not limited to one or more pressure sensors, temperature sensors, humidity sensors, orientation sensors, acoustic sensors, and/or optical sensors. The one or more pressure sensors may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology. The sensor system 128 may be configured to provide an instantaneous reading (e.g. pressure reading) to the controller of the electronics module 120 and/or aggregated readings (e.g. pressure readings) over time. As illustrated in FIGS. 8 and 9, the sensor system 128 may reside outside the flow pathway 119 of the inhaler 100, but may be pneumatically coupled to the flow pathway 119.

The controller of the electronics module 120 may receive signals corresponding to measurements from the sensor system 128. The controller may calculate or determine one or more airflow metrics using the signals received from the sensor system 128. The airflow metrics may be indicative of a profile of airflow through the flow pathway 119 of the inhaler 100. For example, if the sensor system 128 records a change in pressure of 0.3 kilopascals (kPa), the electronics module 120 may determine that the change corresponds to an airflow rate of approximately 45 liters per minute (Lpm) through the flow pathway 119.

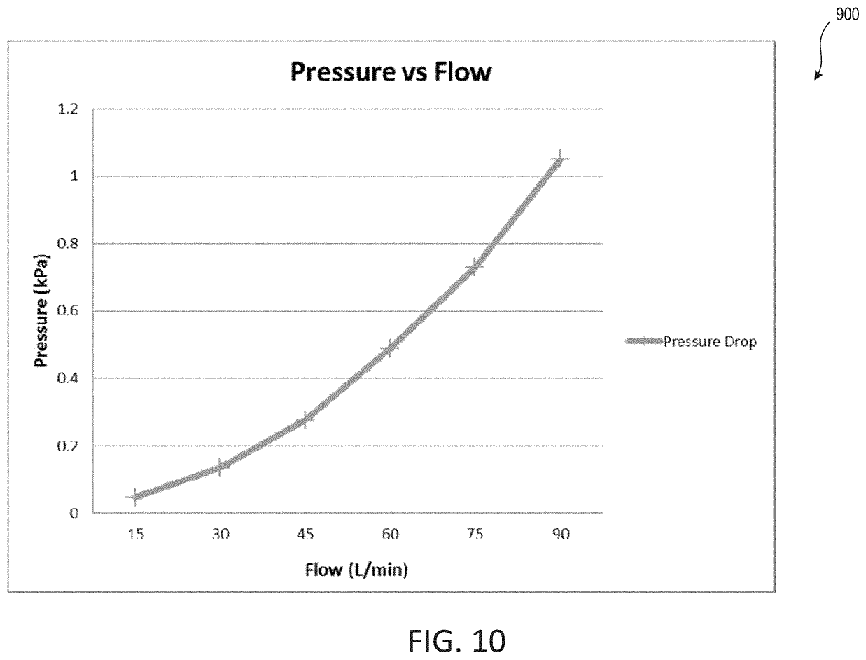

FIG. 10 shows a graph 900 of airflow rates versus pressure. The airflow rates and profile shown in FIG. 10 are merely examples and the determined rates may depend on the size, shape, and design of the inhaler 100 and its components.

The processing module 34 may generate personalized data (e.g., usage events) in real-time by comparing signals received from the sensor system 128 and/or the determined airflow metrics to one or more thresholds or ranges, for example, as part of an assessment of how the inhaler 100 is being used and/or whether the use is likely to result in the delivery of a full dose of medication. For example, where the determined airflow metric corresponds to an inhalation with an airflow rate below a particular threshold, the processing module 34 may determine that there has been no inhalation or an insufficient inhalation from the mouthpiece 106 of the inhaler 100. If the determined airflow metric corresponds to an inhalation with an airflow rate above a particular threshold, the electronics module 120 may determine that there has been an excessive inhalation from the mouthpiece 106. If the determined airflow metric corresponds to an inhalation with an airflow rate within a particular range, the electronics module 120 may determine that the inhalation is “good”, or likely to result in a full dose of medication being delivered.

The pressure measurement readings and/or the computed airflow metrics may be indicative of the quality or strength of inhalation from the inhaler 100. For example, when compared to a particular threshold or range of values, the readings and/or metrics may be used to categorize the inhalation as a certain type of event, such as a good inhalation event, a low inhalation event, a no inhalation event, or an excessive inhalation event. The categorization of the inhalation may be usage parameters stored as personalized data of the subject.

The no inhalation event may be associated with pressure measurement readings and/or airflow metrics below a particular threshold, such as an airflow rate less than or equal to 30 Lpm. The no inhalation event may occur when a subject does not inhale from the mouthpiece 106 after opening the mouthpiece cover 108 and during the measurement cycle. The no inhalation event may also occur when the subject's inspiratory effort is insufficient to ensure proper delivery of the medication via the flow pathway 119, such as when the inspiratory effort generates insufficient airflow to activate the deagglomerator 121 and, thus, aerosolize the medication in the dosing cup 116.

The low inhalation event may be associated with pressure measurement readings and/or airflow metrics within a particular range, such as an airflow rate greater than 30 Lpm and less than or equal to 45 Lpm. The low inhalation event may occur when the subject inhales from the mouthpiece 106 after opening the mouthpiece cover 108 and the subject's inspiratory effort causes at least a partial dose of the medication to be delivered via the flow pathway 119. That is, the inhalation may be sufficient to activate the deagglomerator 121 such that at least a portion of the medication is aerosolized from the dosing cup 116.

The good inhalation event may be associated with pressure measurement readings and/or airflow metrics above the low inhalation event, such as an airflow rate which is greater than 45 Lpm and less than or equal to 200 Lpm. The good inhalation event may occur when the subject inhales from the mouthpiece 106 after opening the mouthpiece cover 108 and the subject's inspiratory effort is sufficient to ensure proper delivery of the medication via the flow pathway 119, such as when the inspiratory effort generates sufficient airflow to activate the deagglomerator 121 and aerosolize a full dose of medication in the dosing cup 116.

The excessive inhalation event may be associated with pressure measurement readings and/or airflow metrics above the good inhalation event, such as an airflow rate above 200 Lpm. The excessive inhalation event may occur when the subject's inspiratory effort exceeds the normal operational parameters of the inhaler 100. The excessive inhalation event may also occur if the device 100 is not properly positioned or held during use, even if the subject's inspiratory effort is within a normal range. For example, the computed airflow rate may exceed 200 Lpm if the air vent is blocked or obstructed (e.g. by a finger or thumb) while the subject is inhaling from the mouthpiece 106.

Any suitable thresholds or ranges may be used to categorize a particular event. Some or all of the events may be used. For example, the no inhalation event may be associated with an airflow rate which is less than or equal to 45 Lpm and the good inhalation event may be associated with an airflow rate which is greater than 45 Lpm and less than or equal to 200 Lpm. As such, the low inhalation event may not be used at all in some cases.

The pressure measurement readings and/or the computed airflow metrics may also be indicative of the direction of flow through the flow pathway 119 of the inhaler 100. For example, if the pressure measurement readings reflect a negative change in pressure, the readings may be indicative of air flowing out of the mouthpiece 106 via the flow pathway 119. If the pressure measurement readings reflect a positive change in pressure, the readings may be indicative of air flowing into the mouthpiece 106 via the flow pathway 119. Accordingly, the pressure measurement readings and/or airflow metrics may be used to determine whether a subject is exhaling into the mouthpiece 106, which may signal that the subject is not using the device 100 properly.

The inhaler 100 may include a spirometer or similarly operating device to enable measurement of lung function metrics. For example, the inhaler 100 may perform measurements to obtain metrics related to a subject's lung capacity. The spirometer or similarly operating device may measure the volume of air inhaled and/or exhaled by the subject. The spirometer or similarly operating device may use pressure transducers, ultrasound, or a water gauge to detect the changes in the volume of air inhaled and/or exhaled.

The personalized data collected from, or calculated based on, the usage of the inhaler 100 (e.g. pressure metrics, airflow metrics, lung function metrics, dose confirmation information, etc.) may be computed and/or assessed via external devices as well (e.g. partially or entirely). More specifically, the wireless communication circuit 129 in the electronics module 120 may include a transmitter and/or receiver (e.g. a transceiver), as well as additional circuitry. The wireless communication circuit 129 may include, or define, the transmission module 14 of the inhaler 100.

For example, the wireless communication circuit 129 may include a Bluetooth chip set (e.g. a Bluetooth Low Energy chip set), a ZigBee chipset, a Thread chipset, etc. As such, the electronics module 120 may wirelessly provide the personalized data, such as pressure measurements, airflow metrics, lung function metrics, dose confirmation information, and/or other conditions related to usage of the inhaler 100, to an external processing module 34, such as a processing module 34 included in a smart phone 40. The personalized data may be provided in real time to the external device to enable exacerbation risk prediction based on real-time data from the inhaler 100 that indicates time of use, how the inhaler 100 is being used, and personalized data about the subject, such as real-time data related to the subject's lung function and/or medical treatment. The external device may include software for processing the received information and for providing compliance and adherence feedback to the subject via a graphical user interface (GUI). The graphical user interface may be included in, or may define, the user interface 38 included in the system 10.

The airflow metrics may include personalized data that is collected from the inhaler 100 in real-time, such as one or more of an average flow of an inhalation/exhalation, a peak flow of an inhalation/exhalation (e.g. a maximum inhalation received), a volume of an inhalation/exhalation, a time to peak of an inhalation/exhalation, and/or the duration of an inhalation/exhalation. The airflow metrics may also be indicative of the direction of flow through the flow pathway 119. That is, a negative change in pressure may correspond to an inhalation from the mouthpiece 106, while a positive change in pressure may correspond to an exhalation into the mouthpiece 106. When calculating the airflow metrics, the electronics module 120 may be configured to eliminate or minimize any distortions caused by environmental conditions. For example, the electronics module 120 may re-zero to account for changes in atmospheric pressure before or after calculating the airflow metrics. The one or more pressure measurements and/or airflow metrics may be timestamped and stored in the memory of the electronics module 120.

In addition to the airflow metrics, the inhaler 100, or another computing device, may use the airflow metrics to generate additional personalized data. For example, the controller of the electronics module 120 of the inhaler 100 may translate the airflow metrics into other metrics that indicate the subject's lung function and/or lung health that are understood to medical practitioners, such as peak inspiratory flow metrics, peak expiratory flow metrics, and/or forced expiratory volume in 1 second (FEV1), for example. The electronics module 120 of the inhaler may determine a measure of the subject's lung function and/or lung health using a mathematical model such as a regression model. The mathematical model may identify a correlation between the total volume of an inhalation and FEV1. The mathematical model may identify a correlation between peak inspiratory flow and FEV1. The mathematical model may identify a correlation between the total volume of an inhalation and peak expiratory flow. The mathematical model may identify a correlation between peak inspiratory flow and peak expiratory flow.

The battery 126 may provide power to the components of the PCB 122. The battery 126 may be any suitable source for powering the electronics module 120, such as a coin cell battery, for example. The battery 126 may be rechargeable or non-rechargeable. The battery 126 may be housed by the battery holder 124. The battery holder 124 may be secured to the PCB 122 such that the battery 126 maintains continuous contact with the PCB 122 and/or is in electrical connection with the components of the PCB 122. The battery 126 may have a particular battery capacity that may affect the life of the battery 126. As will be further discussed below, the distribution of power from the battery 126 to the one or more components of the PCB 122 may be managed to ensure the battery 126 can power the electronics module 120 over the useful life of the inhaler 100 and/or the medication contained therein.

In a connected state, the communication circuit and memory may be powered on and the electronics module 120 may be “paired” with an external device, such as a smart phone. The controller may retrieve data from the memory and wirelessly transmit the data to the external device. The controller may retrieve and transmit the data currently stored in the memory. The controller may also retrieve and transmit a portion of the data currently stored in the memory. For example, the controller may be able to determine which portions have already been transmitted to the external device and then transmit the portion(s) that have not been previously transmitted. Alternatively, the external device may request specific data from the controller, such as any data that has been collected by the electronics module 120 after a particular time or after the last transmission to the external device. The controller may retrieve the specific data, if any, from the memory and transmit the specific data to the external device.

The data stored in the memory of the electronics module 120 (e.g. the signals generated by the switch 130, the pressure measurement readings taken by the sensory system 128 and/or the airflow metrics computed by the controller of the PCB 122) may be transmitted to an external device, which may process and analyze the data to determine the usage parameters associated with the inhaler 100. Further, a mobile application residing on the mobile device may generate feedback for the user based on data received from the electronics module 120. For example, the mobile application may generate daily, weekly, or monthly report, provide confirmation of error events or notifications, provide instructive feedback to the subject, and/or the like.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

What is claimed is:

1. A system for personalized assessment of a user's respiratory health, the system comprising:
a memory comprising compute-executable instructions; and
a processor coupled to the memory, wherein the processor is operative to:
receive a plurality of usage events associated with a plurality of different users, wherein each usage event is associated with an inhaler, a medicament type, and a user of the plurality of different users, and wherein each usage event comprises a time associated with the usage event and one or more inhalation parameters of the usage event, wherein the one or more inhalation parameters comprise a peak inhalation flow (PIF) for the usage event or an inhalation volume for the usage event;
train a machine learning algorithm using training data via an unsupervised learning method, wherein the training data comprises the time and the one or more inhalation parameters associated with each of the plurality of usage events;
determine, using the trained machine learning algorithm, a compliance score for a user;
cause a display device to generate a notification indicating the compliance score for the user.

2. The system of claim 1, wherein the one or more inhalation parameters comprises both the PIF for the usage event and the inhalation volume for the usage event.

3. The system of claim 1, wherein at least a subset of the plurality of usage events are maintenance usage events that are associated with a maintenance medicament type and a dosing schedule for the maintenance medicament type; and
wherein the training data further comprises an adherence ratio that indicates the user's adherence to the dosing schedule for the maintenance medicament type for each user of the plurality of users that is associated with at least one maintenance usage event.

4. The system of claim 3, wherein the adherence is determined based on a comparison between a number of maintenance usage events of the user over a predetermined period of time and a number of maintenance usage events indicated by the dosing schedule for the predetermined period of time.

5. The system of claim 1, wherein at least a subset of the plurality of usage events are rescue usage events that are associated with a rescue medicament type; and
wherein the training data further comprises a user's frequency of rescue usage events for each user of the plurality of users that is associated with at least one rescue usage event.

6. The system of claim 5, wherein the user's frequency of rescue usage events comprises a comparison between a number of rescue usage events of the user over a predetermined period of time and a baseline number of rescue usage events of the user.

7. The system of claim 5, wherein the user's frequency of rescue usage events comprises an average number of daily rescue usage events for the user for a predetermined period of time.

8. The system of claim 5, wherein the user's frequency of rescue usage events comprises an absolute number of rescue usage events for the user for a predetermined period of time.

9. The system of claim 1, wherein a first subset of the plurality of usage events are maintenance usage events that are associated with a maintenance medicament type and a dosing schedule for the maintenance medicament type, and a second subset of the plurality of usage events are rescue usage events that are associated with a rescue medicament type; and
wherein the training data further comprises an adherence ratio that indicates the user's adherence to the dosing schedule for the maintenance medicament type for each user of the plurality of users that is associated with at least one maintenance usage event, and a user's frequency of rescue usage events for each user of the plurality of users that is associated with at least one rescue usage event.

10. The system of claim 1, wherein the training data comprises one or more of:
a number of usage events of a rescue medicament type for a user of the plurality of users in a last predetermined number of days; or
a number of missed usage events of a maintenance medicament type for a user of the plurality of users over the last predetermined number of days

11. The system of claim 1, wherein the training data comprises any combination of:
a percent change in inhalation peak flow for a previous number of usage events for a user of the plurality of users compared to an average inhalation peak flow of the user; or
a percent change in inhalation volume for a previous number of usage events for a user of the plurality of users compared to an average inhalation volume of the user.

12. The system of claim 1, wherein the time associated with each of the plurality of usage events is an indication of whether the usage event occurred during the daytime or nighttime.

13. The system of claim 1, wherein the processor is operative to:
determine an environmental condition for each of the plurality of usage events using a respective time and geographic location associated with the usage event; and
wherein the training data further comprises the environmental condition for each of the plurality of usage events.

14. The system of claim 13, wherein the environmental condition comprises any combination of temperature, humidity, outdoor air pollutants, particulate matter of 2.5 microns or smaller (PM2.5), particulate matter of 10 microns or smaller (PM10), ozone, nitrogen dioxide (NO₂), or sulfur dioxide (SO₂).

15. The system of claim 1, wherein the unsupervised learning method comprises a clustering method.

16. The system of claim 1, wherein the unsupervised learning method comprises a k-means or c-means clustering method.

17. The system of claim 1, wherein the display device is associated with the user or a health care provider of the user.

18. The system of claim 1, wherein the compliance score indicates how compliant the user has been during usage events in a last predetermined number of days.

19. The system of claim 18, wherein the compliance score further indicates how adherent the user has been with respect to a dosing schedule associated with a maintenance medicament.

20. The system of claim 1, wherein the processor is operative to:
determine, using the trained machine learning algorithm, an attribute that the user should improve upon to improve their compliance score; and
cause the display device to generate an attribute notification indicating the attribute for the user.

21. The system of claim 20, wherein the attribute comprises one or more of the following: taking a maintenance medicament at a different time of day, increasing the PIF of future usage events, or increasing inhaled volume of future usage events.

22. The system of claim 20, wherein the processor is operative to:
determine, using the trained machine learning algorithm, a significance factor for each of a plurality of attributes; and
determine, using the trained machine learning algorithm, the attribute that the user should improve upon to improve their compliance score based on the significance factor for each of the plurality of attributes.

23.-96. (canceled)
